SEQUENCE LISTING (40 sequences)

SEQ ID NO: 1 - protein, 500 aa, Saccharomyces cerevisiae
MLRNTLKCAQLSSKYGFKTTTRTFMTTQPQLNVTDAPPVLFTVQDTARVITLNRPKKLNALNAEMSESMFKTLNEYAKSDTTNLVILKSSNRPRSFCAGGDVATVAIFNFNKEFAKSIKFFTDEYSLNFQIATYLKPIVTFMDGITMGGGVGLSIHTPFRIATENTKWAMPEMDIGFFPDVGSTFALPRIVTLANSNSQMALYLCLTGEVVTGADAYMLGLASHYVSSENLDALQKRLGEISPPFNNDPQSAYFFGMVNESIDEFVSPLPKDYVFKYSNEKLNVIEACFNLSKNGTIEDIMNNLRQYEGSAEGKAFAQEIKTKLLTKSPSSLQIALRLVQENSRDHIESAIKRDLYTAANMCMNQDSLVEFSEATKHKLIDKQRVPYPWTKKEQLFVSQLTSITSPKPSLPMSLLRNTSNVTWTQYPYHSKYQLPTEQEIAAYIEKRTNDDTGAKVTEREVLNHFANVIPSRRGKLGIQSLCKIVCERKCEEVNDGLRWK

SEQ ID NO: 2 - DNA, 1000 bp, Haemophilus influenzae
atgttttacactgaaacttatgatgtgattgtgatcggtggtggtcatgcgggtacagaagccgcacttgcaccagctcgtatgggatttaaaacccttttattaacacataatgtagatactttagggcaaatgtcttgtaaccctgcaattggtgggatcggtaaaggtcatttagtaaaagaagtagatgcaatgggcggtttaatggcgcatgctgcagataaagcagggatccaatttcgtactttaaatagcagtaaaggcccagcagtgcgtgctactcgagctcaagctgacagagttctatatcgtcaagctgttcgtactgcattagaaaatcaacctaatttagatattttccaacaagaagcgaccgatattctgattaagcaagatcgagttacaggcgttagcacaaaaatgggattaacttttcgtgctaaatcagtggtattaactgcgggtactttcttagctggtaaaattcatattggtttggaaaattatgaaggtggccgtgcaggggatcctgcttctgtaaatctttcacatcgattaagagatctcggattacgtgtagatcgccttaaaacaggtacaccgccgcgtattgatgcacgtacgatcaattttgatattttagctaaacaacacggtgatgctgttttacctgtgttttcttttatgggatcagttgatgatcaccctcaacaaattccttgttatataactcataccaatgaacaaacccatgaagtgatccgtaataacttggatcgcagtccaatgtatactggtgtgattgaagggatcggtccacgttattgcccatccattgaagataaagtgatgcgtttctcggatcgtaattcacatcaaatttatttagaaccagaaggcttaaccagtaatgaagtgtatccaaacgggatctctaccagtttaccgtttgacgtgcaaatgggcattgtgaattctatgaaaggtttagaaaacg

SEQ ID NO: 3 - protein, 598 aa, Artificial Sequence
  EHD3 EC 3.1.2.4 malonyl-CoA hydrolase consensus sequence
  VARIANT (1)..(16): Xaa can be any naturally occurring amino acid or absent
  VARIANT (18)..(18): Ile, Leu or Val
  VARIANT (19)..(19): Arg, Lys or His
  VARIANT (20)..(21): Ser or Thr
  VARIANT (22)..(22): Ile, Leu or Val
  VARIANT (23)..(23): Arg, Lys or His
  VARIANT (24)..(24): Xaa can be any naturally occurring amino acid
  VARIANT (27)..(27): Xaa can be any naturally occurring amino acid
  VARIANT (28)..(28): Ser or Thr
  VARIANT (29)..(30): Arg, Lys or His
  VARIANT (31)..(31): Xaa can be any naturally occurring amino acid
  VARIANT (32)..(35): Xaa can be any naturally occurring amino acid or absent
  VARIANT (36)..(36): Xaa can be any naturally occurring amino acid
  VARIANT (37)..(40): Xaa can be any naturally occurring amino acid or absent
  VARIANT (41)..(41): Xaa can be any naturally occurring amino acid
  VARIANT (44)..(44): Ile, Leu or Val
  VARIANT (45)..(46): Arg, Lys or His
  VARIANT (47)..(47): Ile, Leu or Val
  VARIANT (48)..(48): Arg, Lys or His
  VARIANT (49)..(52): Xaa can be any naturally occurring amino acid or absent
  VARIANT (53)..(53): Xaa can be any naturally occurring amino acid
  VARIANT (54)..(58): Xaa can be any naturally occurring amino acid or absent
  VARIANT (59)..(59): Xaa can be any naturally occurring amino acid
  VARIANT (60)..(60): Arg, Lys or His
  VARIANT (61)..(61): Ser or Thr
  VARIANT (62)..(62): Ile, Leu or Val
  VARIANT (63)..(65): Ser or Thr
  VARIANT (66)..(90): Xaa can be any naturally occurring amino acid or absent
  VARIANT (93)..(93): Ile, Leu or Val
  VARIANT (95)..(97): Xaa can be any naturally occurring amino acid
  VARIANT (98)..(100): Ser or Thr
  VARIANT (102)..(102): Ile, Leu or Val
  VARIANT (104)..(104): Ser or Thr
  VARIANT (105)..(105): Ile, Leu or Val
  VARIANT (107)..(107): Asp or Glu
  VARIANT (108)..(108): Ser or Thr
  VARIANT (111)..(112): Ile, Leu or Val
  VARIANT (113)..(113): Ser or Thr
  VARIANT (118)..(118): Arg, Lys or His
  VARIANT (125)..(125): Ser or Thr
  VARIANT (126)..(126): Asp or Glu
  VARIANT (128)..(128): Ser or Thr
  VARIANT (129)..(129): Asp or Glu
  VARIANT (130)..(130): Ser or Thr
  VARIANT (131)..(131): Ile, Leu or Val
  VARIANT (133)..(133): Arg, Lys or His
  VARIANT (134)..(134): Ser or Thr
  VARIANT (139)..(139): Ser or Thr
  VARIANT (142)..(142): Asp or Glu
  VARIANT (143)..(144): Ser or Thr
  VARIANT (146)..(149): Ile, Leu or Val
  VARIANT (150)..(150): Arg, Lys or His
  VARIANT (152)..(152): Ser or Thr
  VARIANT (157)..(157): Ser or Thr
  VARIANT (158)..(158): Ile, Leu or Val
  VARIANT (166)..(166): Ser or Thr
  VARIANT (167)..(167): Ile, Leu or Val
  VARIANT (169)..(169): Ile, Leu or Val
  VARIANT (170)..(170): Xaa can be any naturally occurring amino acid
  VARIANT (172)..(172): Ile, Leu or Val
  VARIANT (173)..(173): Asp or Glu
  VARIANT (174)..(175): Arg, Lys or His
  VARIANT (177)..(178): Xaa can be any naturally occurring amino acid or absent
  VARIANT (179)..(180): Arg, Lys or His
  VARIANT (181)..(181): Ser or Thr
  VARIANT (182)..(182): Ile, Leu or Val
  VARIANT (183)..(183): Asp or Glu
  VARIANT (186)..(186): Arg, Lys or His
  VARIANT (187)..(187): Ser or Thr
  VARIANT (188)..(188): Ala, Asp, Arg, His, Lys, Ser, Thr, Asn, Gln, Tyr
  VARIANT (191)..(191): Ile, Leu or Val
  VARIANT (195)..(195): Ile, Leu or Val
  VARIANT (199)..(199): Ile, Leu or Val
  VARIANT (202)..(205): Ile, Leu or Val
  VARIANT (207)..(207): Asp or Glu
  VARIANT (217)..(217): Ile, Leu or Val
  VARIANT (218)..(218): Ser or Thr
  VARIANT (219)..(219): Ile, Leu or Val
  VARIANT (221)..(221): Ser or Thr
  VARIANT (225)..(225): Ile, Leu or Val
  VARIANT (231)..(231): Arg, Lys or His
  VARIANT (247)..(248): Ser or Thr
  VARIANT (251)..(251): Ile, Leu or Val
  VARIANT (253)..(253): Arg, Lys or His
  VARIANT (254)..(255): Ile, Leu or Val
  VARIANT (256)..(256): Ser or Thr
  VARIANT (257)..(257): Ile, Leu or Val
  VARIANT (260)..(260): Ser or Thr
  VARIANT (261)..(261): Arg, Lys or His
  VARIANT (262)..(262): Ser or Thr
  VARIANT (264)..(264): Ile, Leu or Val
  VARIANT (266)..(266): Ile, Leu or Val
  VARIANT (270)..(270): Ile, Leu or Val
  VARIANT (273)..(273): Asp or Glu
  VARIANT (274)..(275): Ile, Leu or Val
  VARIANT (276)..(276): Ser or Thr
  VARIANT (278)..(279): Asp or Glu
  VARIANT (282)..(283): Ile, Leu or Val
  VARIANT (285)..(285): Ile, Leu or Val
  VARIANT (290)..(290): Ile, Leu or Val
  VARIANT (291)..(291): Ser or Thr
  VARIANT (292)..(292): Arg, Lys or His
  VARIANT (293)..(293): Asp or Glu
  VARIANT (295)..(295): Ile, Leu or Val
  VARIANT (296)..(297): Asp or Glu
  VARIANT (299)..(299): Asp or Glu
  VARIANT (300)..(300): Arg, Lys or His
  VARIANT (305)..(305): Ile, Leu or Val
  VARIANT (306)..(306): Arg, Lys or His
  VARIANT (308)..(308): Ser or Thr
  VARIANT (309)..(309): Asp or Glu
  VARIANT (310)..(310): Xaa can be any naturally occurring amino acid
  VARIANT (311)..(311): Ile, Leu or Val
  VARIANT (312)..(312): Asp or Glu
  VARIANT (313)..(313): Xaa can be any naturally occurring amino acid
  VARIANT (314)..(314): Xaa can be any naturally occurring amino acid or absent
  VARIANT (315)..(317): Xaa can be any naturally occurring amino acid
  VARIANT (318)..(318): Ser or Thr
  VARIANT (320)..(320): Ser or Thr
  VARIANT (321)..(322): Asp or Glu
  VARIANT (325)..(325): Asp or Glu
  VARIANT (326)..(327): Ile, Leu or Val
  VARIANT (329)..(329): Asp or Glu
  VARIANT (330)..(330): Ser or Thr
  VARIANT (331)..(331): Ile, Leu or Val
  VARIANT (332)..(332): Asp or Glu
  VARIANT (335)..(336): Ser or Thr
  VARIANT (338)..(338): Xaa can be any naturally occurring amino acid or absent
  VARIANT (339)..(339): Ile, Leu or Val
  VARIANT (341)..(341): Arg, Lys or His
  VARIANT (342)..(342): Asp or Glu
  VARIANT (344)..(344): Arg, Lys or His
  VARIANT (346)..(346): Arg, Lys or His
  VARIANT (348)..(348): Ser or Thr
  VARIANT (350)..(350): Asp or Glu
  VARIANT (351)..(351): Arg, Lys or His
  VARIANT (353)..(353): Asp or Glu
  VARIANT (356)..(356): Asp or Glu
  VARIANT (357)..(357): Arg, Lys or His
  VARIANT (360)..(360): Asp or Glu
  VARIANT (361)..(361): Ile, Leu or Val
  VARIANT (362)..(362): Ser or Thr
  VARIANT (363)..(363): Arg, Lys or His
  VARIANT (364)..(364): Ile, Leu or Val
  VARIANT (365)..(366): Ser or Thr
  VARIANT (367)..(367): Ile, Leu or Val
  VARIANT (368)..(368): Arg, Lys or His
  VARIANT (369)..(369): Asp or Glu
  VARIANT (370)..(371): Ile, Leu or Val
  VARIANT (372)..(373): Arg, Lys or His
  VARIANT (375)..(376): Asp or Glu
  VARIANT (377)..(377): Ile, Leu or Val
  VARIANT (378)..(378): Xaa can be any naturally occurring amino acid or absent
  VARIANT (379)..(380): Xaa can be any naturally occurring amino acid
  VARIANT (381)..(381): Xaa can be any naturally occurring amino acid or absent
  VARIANT (383)..(383): Asp or Glu
  VARIANT (385)..(385): Ser or Thr
  VARIANT (386)..(387): Asp or Glu
  VARIANT (389)..(389): Arg, Lys or His
  VARIANT (390)..(390): Asp or Glu
  VARIANT (393)..(393): Arg, Lys or His
  VARIANT (394)..(394): Asp or Glu
  VARIANT (395)..(395): Ile, Leu or Val
  VARIANT (396)..(396): Arg, Lys or His
  VARIANT (397)..(397): Asp or Glu
  VARIANT (398)..(398): Arg, Lys or His
  VARIANT (400)..(400): Ile, Leu or Val
  VARIANT (401)..(401): Ser or Thr
  VARIANT (405)..(405): Ser or Thr
  VARIANT (407)..(407): Ile, Leu or Val
  VARIANT (409)..(409): Ile, Leu or Val
  VARIANT (411)..(411): Ile, Leu or Val
  VARIANT (412)..(412): Arg, Lys or His
  VARIANT (413)..(414): Ile, Leu or Val
  VARIANT (415)..(415): Arg, Lys or His
  VARIANT (416)..(416): Asp or Glu
  VARIANT (418)..(418): Ser or Thr
  VARIANT (419)..(419): Arg, Lys or His
  VARIANT (420)..(420): Asp or Glu
  VARIANT (421)..(421): Arg, Lys or His
  VARIANT (422)..(422): Ile, Leu or Val
  VARIANT (423)..(423): Asp or Glu
  VARIANT (424)..(424): Ser or Thr
  VARIANT (426)..(426): Ile, Leu or Val
  VARIANT (427)..(428): Arg, Lys or His
  VARIANT (431)..(431): Ile, Leu or Val
  VARIANT (434)..(434): Ser or Thr
  VARIANT (438)..(438): Ile, Leu or Val
  VARIANT (440)..(440): Xaa can be any naturally occurring amino acid or absent
  VARIANT (441)..(443): Xaa can be any naturally occurring amino acid
  VARIANT (445)..(445): Asp or Glu
  VARIANT (446)..(446): Ser or Thr
  VARIANT (447)..(448): Ile, Leu or Val
  VARIANT (451)..(451): Ser or Thr
  VARIANT (452)..(452): Asp or Glu
  VARIANT (454)..(454): Ser or Thr
  VARIANT (455)..(456): Arg, Lys or His
  VARIANT (459)..(459): Ile, Leu or Val
  VARIANT (460)..(460): Asp or Glu
  VARIANT (463)..(463): Arg, Lys or His
  VARIANT (464)..(464): Ile, Leu or Val
  VARIANT (464)..(464): Ile, Leu or Val
  VARIANT (469)..(471): Arg, Lys or His
  VARIANT (472)..(472): Xaa can be any naturally occurring amino acid
  VARIANT (473)..(474): Asp or Glu
  VARIANT (475)..(475): Ile, Leu or Val
  VARIANT (476)..(476): Ser or Thr
  VARIANT (477)..(477): Ile, Leu or Val
  VARIANT (478)..(478): Ser or Thr
  VARIANT (480)..(480): Ile, Leu or Val
  VARIANT (481)..(482): Ser or Thr
  VARIANT (483)..(484): Ile, Leu or Val
  VARIANT (485)..(485): Ser or Thr
  VARIANT (487)..(487): Arg, Lys or His
  VARIANT (489)..(489): Ser or Thr
  VARIANT (490)..(490): Ile, Leu or Val
  VARIANT (492)..(492): Ile, Leu or Val
  VARIANT (493)..(493): Ser or Thr
  VARIANT (495)..(495): Ile, Leu or Val
  VARIANT (496)..(496): Arg, Lys or His
  VARIANT (498)..(499): Ser or Thr
  VARIANT (501)..(501): Ile, Leu or Val
  VARIANT (504)..(504): Arg, Lys or His
  VARIANT (505)..(505): Asp or Glu
  VARIANT (508)..(509): Arg, Lys or His
  VARIANT (510)..(510): Ile, Leu or Val
  VARIANT (511)..(511): Arg, Lys or His
  VARIANT (516)..(516): Ser or Thr
  VARIANT (517)..(517): Asp or Glu
  VARIANT (518)..(518): Ser or Thr
  VARIANT (519)..(519): Asp or Glu
  VARIANT (520)..(520): Ile, Leu or Val
  VARIANT (521)..(521): Arg, Lys or His
  VARIANT (524)..(524): Ile, Leu or Val
  VARIANT (525)..(527): Arg, Lys or His
  VARIANT (528)..(528): Asp or Glu
  VARIANT (530)..(530): Arg, Lys or His
  VARIANT (532)..(534): Xaa can be any naturally occurring amino acid or absent
  VARIANT (535)..(535): Xaa can be any naturally occurring amino acid
  VARIANT (537)..(537): Xaa can be any naturally occurring amino acid or absent
  VARIANT (538)..(539): Xaa can be any naturally occurring amino acid
  VARIANT (540)..(540): Ile, Leu or Val
  VARIANT (541)..(541): Arg, Lys or His
  VARIANT (542)..(542): Ile, Leu or Val
  VARIANT (543)..(543): Ser or Thr
  VARIANT (543)..(543): Ser or Thr
  VARIANT (544)..(545): Arg, Lys, or His
  VARIANT (546)..(546): Asp or Glu
  VARIANT (547)..(548): Ile, Leu or Val
  VARIANT (549)..(550): Arg, Lys or His
  VARIANT (552)..(552): Ser or Thr
  VARIANT (554)..(554): Ile, Leu or Val
  VARIANT (556)..(556): Asp or Glu
  VARIANT (557)..(557): Ser or Thr
  VARIANT (558)..(559): Arg, Lys or His
  VARIANT (560)..(560): Asp or Glu
  VARIANT (562)..(562): Ile, Leu or Val
  VARIANT (564)..(564): Ile, Leu or Val
  VARIANT (565)..(565): Asp or Glu
  VARIANT (566)..(566): Ser or Thr
  VARIANT (567)..(568): Ile, Leu or Val
  VARIANT (569)..(569): Arg, Lys or His
  VARIANT (570)..(572): Ile, Leu or Val
  VARIANT (573)..(573): Asp or Glu
  VARIANT (574)..(575): Arg, Lys or His
  VARIANT (577)..(577): Ser or Thr
  VARIANT (578)..(580): Asp or Glu
  VARIANT (582)..(582): Xaa can be any naturally occurring amino acid
  VARIANT (585)..(585): Ile, Leu or Val
  VARIANT (586)..(586): Arg, Lys or His
  VARIANT (588)..(588): Arg, Lys or His
  VARIANT (589)..(590): Xaa can be any naturally occurring amino acid
  VARIANT (591)..(591): Xaa can be any naturally occurring amino acid or absent
  VARIANT (592)..(592): Xaa can be any naturally occurring amino acid
  VARIANT (593)..(598): Xaa can be any naturally occurring amino acid or absent
XXXXXXXXXXXXXXXXMXXXXXXXAQXXXXXXXXXXXXXXXGFXXXXXXXXXXXXXXXXXXXXXXXXXXXXXXXXXXXXXXXXXXXXXXXAQXNXXXXXXVXFXXQXXARXXXLNRPXKLNALNXXMXXXXFXXLNEYXKSXXXNXXXXXSXNQPRXXCAGGDVAXXAXXNXXXXFXXXXXXXFFXXXYSXNFQXATYXKPXXXXMXGITMGGGVGXXXHXPFRXATENTXWAMPEMDIGFFPDVGXXFAXPXXXXXANXXXQXAXYLCXTGXXXXGXXAYXXGXASHYXXXXNXXXLXXRLGEXXPXXXXXXXXXXXQXXXFFXXXNXXXXEFXXPXXPXXYXFXYXNXXLXVIXXCFXXXXXXXXXXXXXXLXXXXXXXYXGXXXAXXFAXXXXXXLXXKSPXSXQXAXXXXXXNXXXXXXXAXXXDLXTAXNMCXNXXXXQXXXXEFXXAXXXKLXXKQXXPYPWXXXXXXXXXXQXXXXXXPXPXXPXXLXXNXXNXTWXXYPXXXXYQLPXXXXXXQYXXXXXNXNXXXXGXXXXXXXXXXXXXXFXNXNXXXXXKXGXXXXXXXXXXXXCXXXXAXGGXXWXXXXXXXXXXX

SEQ ID NO: 4 - protein, 351 aa, Artificial Sequence
  Bacillus EC 3.1.2.4 malonyl-CoA hydrolase consensus sequence
  VARIANT (4)..(4): Arg, Lys or His
  VARIANT (9)..(9): Ile, Leu or Val
  VARIANT (10)..(10): Ser or Thr
  VARIANT (11)..(11): Asp or Glu
  VARIANT (16)..(16): Ser or Thr
  VARIANT (31)..(31): Asp or Glu
  VARIANT (45)..(45): Arg, Lys or His
  VARIANT (46)..(47): Asp or Glu
  VARIANT (48)..(48): Arg, Lys or His
  VARIANT (51)..(51): Ile, Leu or Val
  VARIANT (59)..(59): Ser or Thr
  VARIANT (79)..(79): Ile, Leu or Val
  VARIANT (83)..(83): Arg, Lys or His
  VARIANT (86)..(86): Arg, Lys or His
  VARIANT (89)..(89): Asp or Glu
  VARIANT (91)..(91): Ala, Asp, Arg, His, Lys, Ser, Thr, Asn, Gln, Tyr
  VARIANT (93)..(93): Asp or Glu
  VARIANT (98)..(98): Ile, Leu or Val
  VARIANT (102)..(102): Arg, Lys or His
  VARIANT (125)..(125): Arg, Lys or His
  VARIANT (131)..(131): Asp or Glu
  VARIANT (132)..(132): Arg, Lys or His
  VARIANT (156)..(156): Arg, Lys or His
  VARIANT (161)..(161): Ser or Thr
  VARIANT (163)..(163): Arg, Lys or His
  VARIANT (168)..(168): Ser or Thr
  VARIANT (170)..(170): Ser or Thr
  VARIANT (171)..(171): Ile, Leu or Val
  VARIANT (175)..(175): Ser or Thr
  VARIANT (188)..(189): Ser or Thr
  VARIANT (190)..(190): Asp or Glu
  VARIANT (191)..(191): Ser or Thr
  VARIANT (194)..(194): Arg, Lys or His
  VARIANT (197)..(197): Ser or Thr
  VARIANT (198)..(198): Asp or Glu
  VARIANT (199)..(199): Ile, Leu or Val
  VARIANT (200)..(200): Asp or Glu
  VARIANT (200)..(200): Ser or Thr
  VARIANT (201)..(201): Ser or Thr
  VARIANT (202)..(202): Ile, Leu or Val
  VARIANT (205)..(206): Arg, Lys or His
  VARIANT (207)..(209): Asp or Glu
  VARIANT (211)..(211): Arg, Lys or His
  VARIANT (212)..(212): Ser or Thr
  VARIANT (213)..(213): Arg, Lys or His
  VARIANT (216)..(216): Asp or Glu
  VARIANT (217)..(218): Ile, Leu or Val
  VARIANT (219)..(219): Arg, Lys or His
  VARIANT (220)..(220): Ser or Thr
  VARIANT (223)..(226): Ser or Thr
  VARIANT (227)..(227): Ile, Leu or Val
  VARIANT (228)..(228): Asp or Glu
  VARIANT (229)..(229): Ser or Thr
  VARIANT (230)..(230): Asp or Glu
  VARIANT (232)..(233): Ser or Thr
  VARIANT (234)..(235): Ile, Leu or Val
  VARIANT (237)..(237): Asp or Glu
  VARIANT (238)..(238): Ile, Leu or Val
  VARIANT (240)..(240): Ser or Thr
  VARIANT (245)..(245): Asp or Glu
  VARIANT (246)..(246): Ser or Thr
  VARIANT (247)..(247): Ile, Leu or Val
  VARIANT (249)..(249): Asp or Glu
  VARIANT (252)..(252): Arg, Lys or His
  VARIANT (256)..(256): Arg, Lys or His
  VARIANT (257)..(257): Asp or Glu
  VARIANT (259)..(260): Ser or Thr
  VARIANT (263)..(263): Ile, Leu or Val
  VARIANT (264)..(264): Arg, Lys or His
  VARIANT (265)..(265): Ser or Thr
  VARIANT (266)..(266): Arg, Lys or His
  VARIANT (267)..(267): Asp or Glu
  VARIANT (268)..(268): Ser or Thr
  VARIANT (275)..(275): Ile, Leu or Val
  VARIANT (285)..(285): Ile, Leu or Val
  VARIANT (286)..(286): Asp or Glu
  VARIANT (288)..(288): Arg, Lys or His
  VARIANT (289)..(289): Asp or Glu
  VARIANT (292)..(292): Ile, Leu or Val
  VARIANT (294)..(294): Asp or Glu
  VARIANT (301)..(301): Ile, Leu or Val
  VARIANT (310)..(310): Asp or Glu
  VARIANT (319)..(319): Ile, Leu or Val
  VARIANT (321)..(321): Ile, Leu or Val
  VARIANT (332)..(332): Arg, Lys or His
  VARIANT (334)..(334): Ile, Leu or Val
  VARIANT (335)..(335): Ser or Thr
  VARIANT (338)..(338): Ser or Thr
  VARIANT (339)..(341): Asp or Glu
  VARIANT (344)..(344): Arg, Lys or His
MTEXVLFSXXXNGVAXITLNRPKALNSLSYXMLQPIGQKLKEWEXXXXIAXIVLKGAGXKGFCAGGDIKTLYEARSNEXALQXAEXFFXEXYXIDTYXYQYXKPIIACLDGIVMGGGVGLTNGAXYRIVTXXTKWAMPEMNIGFFPDVGAAYFLNXAPGYXGXYVALXAXXLKAXDVLFINAADYFMXXXXLPXFLXXXXXXNWXXXXXVXXXLKXXXXXFAXXXXXXXXLXXXXEXXNXHFAFXXXEXIIXSLEXXQXXFAXXXXXXLLSKSPXSLKVTLKQFXXGXXKSXEXCFATDLXLAKNFMRHXDFFEGVRSXVXDKDQNPNYKYXQXXDVXXXXVNXFFNLLNA

SEQ ID NO: 5 - protein, 370 aa, Artificial Sequence
  Pseudomonas EC 3.1.2.4 malonyl-CoA hydrolase consensus sequence
  VARIANT (3)..(3): Ile, Leu or Val
  VARIANT (4)..(4): Arg, Lys or His
  VARIANT (7)..(7): Asp or Glu
  VARIANT (8)..(8): Arg, Lys or His
  VARIANT (9)..(10): Ser or Thr
  VARIANT (11)..(11): Ile, Leu or Val
  VARIANT (12)..(12): Arg, Lys or His
  VARIANT (18)..(18): Ile, Leu or Val
  VARIANT (20)..(20): Ser or Thr
  VARIANT (24)..(24): Asp or Glu
  VARIANT (25)..(25): Arg, Lys or His
  VARIANT (26)..(26): Ser or Thr
  VARIANT (31)..(31): Ser or Thr
  VARIANT (36)..(36): Asp or Glu
  VARIANT (37)..(37): Ile, Leu or Val
  VARIANT (40)..(40): Asp or Glu
  VARIANT (41)..(41): Arg, Lys or His
  VARIANT (42)..(42): Ile, Leu or Val
  VARIANT (43)..(43): Arg, Lys or His
  VARIANT (47)..(47): Arg, Lys or His
  VARIANT (48)..(48): Asp or Glu
  VARIANT (51)..(52): Ile, Leu or Val
  VARIANT (55)..(55): Ile, Leu or Val
  VARIANT (69)..(69): Asp or Glu
  VARIANT (71)..(72): Arg, Lys or His
  VARIANT (74)..(74): Ile, Leu or Val
  VARIANT (75)..(75): Asp or Glu
  VARIANT (78)..(78): Arg, Lys or His
  VARIANT (79)..(79): Asp or Glu
  VARIANT (80)..(80): Arg, Lys or His
  VARIANT (83)..(83): Asp or Glu
  VARIANT (84)..(84): Ile, Leu or Val
  VARIANT (90)..(90): Arg, Lys or His
  VARIANT (94)..(94): Asp or Glu
  VARIANT (95)..(95): Ala, Asp, Arg, His, Lys, Ser, Thr, Asn, Gln, or Tyr
  VARIANT (99)..(99): Asp or Glu
  VARIANT (100)..(100): Arg, Lys or His
  VARIANT (101)..(101): Arg, Lys or His
  VARIANT (102)..(102): Ile, Leu or Val
  VARIANT (104)..(104): Ser or Thr
  VARIANT (109)..(110): Ile, Leu or Val
  VARIANT (116)..(116): Arg, Lys or His
  VARIANT (118)..(118): Ile, Leu or Val
  VARIANT (125)..(125): Ile, Leu or Val
  VARIANT (129)..(129): Ser or Thr
  VARIANT (130)..(130): Ile, Leu or Val
  VARIANT (136)..(137): Ser or Thr
  VARIANT (144)..(144): Ile, Leu or Val
  VARIANT (145)..(145): Ser or Thr
  VARIANT (159)..(159): Ser or Thr
  VARIANT (161)..(161): Ile, Leu or Val
  VARIANT (164)..(164): Arg, Lys or His
  VARIANT (169)..(169): Ile, Leu or Val
  VARIANT (172)..(172): Ser or Thr
  VARIANT (175)..(175): Arg, Lys or His
  VARIANT (176)..(176): Ile, Leu or Val
  VARIANT (179)..(179): Arg, Lys or His
  VARIANT (182)..(182): Ile, Leu or Val
  VARIANT (185)..(185): Asp or Glu
  VARIANT (191)..(191): Ile, Leu or Val
  VARIANT (192)..(192): Arg, Lys or His
  VARIANT (193)..(193): Asp or Glu
  VARIANT (194)..(194): Arg, Lys or His
  VARIANT (197)..(198): Asp or Glu
  VARIANT (200)..(200): Ile, Leu or Val
  VARIANT (201)..(202): Asp or Glu
  VARIANT (204)..(204): Ile, Leu or Val
  VARIANT (206)..(206): Ile, Leu or Val
  VARIANT (212)..(212): Ser or Thr
  VARIANT (213)..(213): Asp or Glu
  VARIANT (214)..(214): Ile, Leu or Val
  VARIANT (217)..(217): Arg, Lys or His
  VARIANT (220)..(220): Ile, Leu or Val
  VARIANT (221)..(221): Arg, Lys or His
  VARIANT (223)..(224): Asp or Glu
  VARIANT (225)..(225): Arg, Lys or His
  VARIANT (226)..(226): Arg, Lys or His
  VARIANT (228)..(228): Arg, Lys or His
  VARIANT (229)..(230): Asp or Glu
  VARIANT (231)..(231): Ile, Leu or Val
  VARIANT (233)..(233): Asp or Glu
  VARIANT (236)..(236): Ile, Leu or Val
  VARIANT (242)..(242): Arg, Lys or His
  VARIANT (243)..(243): Ile, Leu or Val
  VARIANT (245)..(245): Asp or Glu
  VARIANT (246)..(246): Ile, Leu or Val
  VARIANT (249)..(249): Ile, Leu or Val
  VARIANT (251)..(251): Asp or Glu
  VARIANT (252)..(252): Ile, Leu or Val
  VARIANT (254)..(254): Ser or Thr
  VARIANT (257)..(257): Arg, Lys or His
  VARIANT (259)..(259): Ile, Leu or Val
  VARIANT (260)..(260): Asp or Glu
  VARIANT (262)..(262): Ile, Leu or Val
  VARIANT (263)..(263): Arg, Lys or His
  VARIANT (264)..(264): Asp or Glu
  VARIANT (265)..(265): Arg, Lys or His
  VARIANT (266)..(266): Asp or Glu
  VARIANT (270)..(270): Ile, Leu or Val
  VARIANT (272)..(272): Arg, Lys or His
  VARIANT (275)..(276): Arg, Lys or His
  VARIANT (277)..(277): Ile, Leu or Val
  VARIANT (278)..(278): Arg, Lys or His
  VARIANT (279)..(279): Asp or Glu
  VARIANT (283)..(283): Ile, Leu or Val
  VARIANT (284)..(284): Ser or Thr
  VARIANT (286)..(286): Arg, Lys or His
  VARIANT (287)..(287): Ile, Leu or Val
  VARIANT (290)..(290): Asp or Glu
  VARIANT (292)..(292): Ile, Leu or Val
  VARIANT (293)..(293): Arg, Lys or His
  VARIANT (297)..(297): Arg, Lys or His
  VARIANT (302)..(302): Asp or Glu
  VARIANT (303)..(303): Ile, Leu or Val
  VARIANT (305)..(305): Arg, Lys or His
  VARIANT (309)..(309): Ser or Thr
  VARIANT (310)..(310): Ile, Leu or Val
  VARIANT (319)..(319): Asp or Glu
  VARIANT (321)..(321): Ser or Thr
  VARIANT (329)..(329): Ile, Leu or Val
  VARIANT (331)..(331): Arg, Lys or His
  VARIANT (333)..(333): Arg, Lys or His
  VARIANT (336)..(336): Arg, Lys or His
  VARIANT (338)..(338): Arg, Lys or His
  VARIANT (341)..(341): Asp or Glu
  VARIANT (342)..(342): Ile, Leu or Val
  VARIANT (345)..(345): Ile, Leu or Val
  VARIANT (347)..(347): Asp or Glu
  VARIANT (349)..(350): Ile, Leu or Val
  VARIANT (350)..(350): Ile, Leu or Val
  VARIANT (351)..(351): Asp or Glu
  VARIANT (355)..(355): Asp or Glu
  VARIANT (356)..(356): Arg, Lys or His
  VARIANT (357)..(357): Ile, Leu or Val
  VARIANT (357)..(357): Ile, Leu or Val
  VARIANT (359)..(359): Asp or Glu
  VARIANT (361)..(362): Arg, Lys or His
  VARIANT (364)..(364): Ile, Leu or Val
  VARIANT (364)..(364): Ile, Leu or Val
  VARIANT (366)..(366): Asp or Glu
  VARIANT (367)..(367): Ile, Leu or Val
  VARIANT (367)..(367): Ile, Leu or Val
  VARIANT (368)..(368): Ser or Thr
  VARIANT (369)..(370): Xaa can be any naturally occurring amino acid
MNXXFEXXXXXXGARIGXAXLDAXXXLNALXLPMIXXLGXXXXAWAXXPGXXCVXLRGNGAKAFCAGGXVXXLXXACXXXPGXXPPLAAXFFAXXYRLXXXXHXYPKPXXCWGHGXVXGGGMGLXQGAXXRIVTPXXRLAMPEXXIGLYPDVGASWFLXRXPGXLGLFXGLXGAXXNAXDAXDLXLADRFXXXXQQXXLXXXLXQXNWQEQXXXQLXSLXXAXXXXAXXXXPXAQXLPRRQXXDXXLDXAXXAXAWXAXXAXXXXXDPLXAXAAXXXXXGCPXXAXXVWXQXXRARXLSLAXXFXMEYXXSLNCCRHPXFXEGVRARLXDXDXQPXWXWPXXAQXPXAXXXAHFXXXWXGXXPXAXXXXX

SEQ ID NO: 6 - protein, 505 aa, Artificial Sequence
  General bacterial EC 3.1.2.4 malonyl-CoA hydrolase consensus sequence
  VARIANT (1)..(10): Xaa can be any naturally occurring amino acid or absent
  VARIANT (11)..(11): Xaa can be any naturally occurring amino acid
  VARIANT (12)..(23): Xaa can be any naturally occurring amino acid or absent
  VARIANT (24)..(24): Xaa can be any naturally occurring amino acid
  VARIANT (25)..(30): Xaa can be any naturally occurring amino acid or absent
  VARIANT (32)..(32): Xaa can be any naturally occurring amino acid
  VARIANT (34)..(35): Xaa can be any naturally occurring amino acid or absent
  VARIANT (39)..(40): Ile, Leu or Val
  VARIANT (42)..(42): Ser or Thr
  VARIANT (43)..(43): Ile, Leu or Val
  VARIANT (48)..(48): Ile, Leu or Val
  VARIANT (52)..(52): Ser or Thr
  VARIANT (57)..(57): Arg, Lys or His
  VARIANT (63)..(63): Ser or Thr
  VARIANT (67)..(67): Ile, Leu or Val
  VARIANT (70)..(70): Ile, Leu or Val
  VARIANT (73)..(73): Arg, Lys or His
  VARIANT (74)..(74): Ile, Leu or Val
  VARIANT (75)..(75): Arg, Lys or His
  VARIANT (80)..(80): Asp or Glu
  VARIANT (83)..(83): Ile, Leu or Val
  VARIANT (86)..(87): Ile, Leu or Val
  VARIANT (89)..(89): Arg, Lys or His
  VARIANT (90)..(90): Xaa can be any naturally occurring amino acid or absent
  VARIANT (95)..(95): Arg, Lys or His
  VARIANT (102)..(102): Asp or Glu
  VARIANT (103)..(103): Ile, Leu or Val
  VARIANT (104)..(104): Arg, Lys or His
  VARIANT (105)..(105): Ser or Thr
  VARIANT (106)..(106): Ile, Leu or Val
  VARIANT (108)..(108): Asp or Glu
  VARIANT (116)..(116): Xaa can be any naturally occurring amino acid
  VARIANT (127)..(127): Asp or Glu
  VARIANT (128)..(128): Xaa can be any naturally occurring amino acid
  VARIANT (128)..(128): Ala, Asp, Arg, His, Lys, Ser, Thr, Asn, Glu, Tyr
  VARIANT (131)..(131): Ile, Leu or Val
  VARIANT (132)..(132): Asp or Glu
  VARIANT (135)..(135): Ile, Leu or Val
  VARIANT (142)..(143): Ile, Leu or Val
  VARIANT (150)..(150): Ile, Leu or Val
  VARIANT (164)..(164): Arg, Lys or His
  VARIANT (165)..(166): Ile, Leu or Val
  VARIANT (170)..(170): Ser or Thr
  VARIANT (171)..(171): Arg, Lys or His
  VARIANT (193)..(193): Arg, Lys or His
  VARIANT (195)..(200): Xaa can be any naturally occurring amino acid or absent
  VARIANT (208)..(208): Ile, Leu or Val
  VARIANT (211)..(211): Ser or Thr
  VARIANT (215)..(215): Ile, Leu or Val
  VARIANT (221)..(221): Ile, Leu or Val
  VARIANT (223)..(223): Ile, Leu or Val
  VARIANT (227)..(227): Asp or Glu
  VARIANT (233)..(233): Asp or Glu
  VARIANT (239)..(239): Ile, Leu or Val
  VARIANT (242)..(242): Ile, Leu or Val
  VARIANT (245)..(245): Ile, Leu or Val
  VARIANT (252)..(252): Asp or Glu
  VARIANT (253)..(253): Ile, Leu or Val
  VARIANT (261)..(261): Xaa can be any naturally occurring amino acid
  VARIANT (262)..(262): Xaa can be any naturally occurring amino acid or absent
  VARIANT (263)..(263): Xaa can be any naturally occurring amino acid
  VARIANT (265)..(265): Ile, Leu or Val
  VARIANT (275)..(275): Asp or Glu
  VARIANT (278)..(278): Ile, Leu or Val
  VARIANT (281)..(284): Xaa can be any naturally occurring amino acid or absent
  VARIANT (289)..(289): Ile, Leu or Val
  VARIANT (296)..(298): Xaa can be any naturally occurring amino acid or absent
  VARIANT (301)..(301): Ile, Leu or Val
  VARIANT (306)..(306): Xaa can be any naturally occurring amino acid
  VARIANT (308)..(308): Ile, Leu or Val
  VARIANT (311)..(311): Ile, Leu or Val
  VARIANT (313)..(314): Xaa can be any naturally occurring amino acid or absent
  VARIANT (316)..(316): Asp or Glu
  VARIANT (328)..(328): Ile, Leu or Val
  VARIANT (332)..(332): Ser or Thr
  VARIANT (334)..(334): Ile, Leu or Val
  VARIANT (335)..(335): Ser or Thr
  VARIANT (337)..(337): Arg, Lys or His
  VARIANT (338)..(338): Ile, Leu or Val
  VARIANT (350)..(350): Ser or Thr
  VARIANT (351)..(351): Ile, Leu or Val
  VARIANT (353)..(353): Asp or Glu
  VARIANT (358)..(358): Asp or Glu
  VARIANT (361)..(361): Ile, Leu or Val
  VARIANT (367)..(368): Arg, Lys or His
  VARIANT (369)..(372): Xaa can be any naturally occurring amino acid or absent
  VARIANT (374)..(374): Asp or Glu
  VARIANT (379)..(379): Ile, Leu or Val
  VARIANT (380)..(380): Arg, Lys or His
  VARIANT (383)..(384): Ile, Leu or Val
  VARIANT (386)..(386): Arg, Lys or His
  VARIANT (387)..(387): Asp or Glu
  VARIANT (393)..(393): Arg, Lys or His
  VARIANT (397)..(397): Ile, Leu or Val
  VARIANT (400)..(400): Ile, Leu or Val
  VARIANT (402)..(402): Asp or Glu
  VARIANT (405)..(419): Xaa can be any naturally occurring amino acid or absent
  VARIANT (420)..(420): Ile, Leu or Val
  VARIANT (427)..(427): Ile, Leu or Val
  VARIANT (431)..(431): Xaa can be any naturally occurring amino acid
  VARIANT (433)..(434): Xaa can be any naturally occurring amino acid or absent
  VARIANT (440)..(440): Xaa can be any naturally occurring amino acid
  VARIANT (441)..(446): Xaa can be any naturally occurring amino acid or absent
  VARIANT (447)..(448): Xaa can be any naturally occurring amino acid
  VARIANT (449)..(505): Xaa can be any naturally occurring amino acid or absent
XXXXXXXXXXXXXXXXXXXXXXXXXXXXXXMXMXXTEHXXFXXSENGXASIXLNRPXALNSLXYDMXQPXGQXXXEWENXERXALXXLXXGAGTXGFCAGGXXXXXYXARSNEPGXALQHAERFFEXXYEXXTYXYQYKKPXXACLDGIXMGGGVGLTNGAKYXXXTERXXWAMPEMNIGFFPDVGAAYFLNXAXXXXXXPGYLGRYXALXASIXKASDVXFXNAAXYFMTSXSLPAFXTEXESXNWHKEDXXHTHLLKEXXXVXRTFATAPNLXSEXAPXXXXSLEEXNSHFAFXXXDTXEEIWXAXHSXEXXKXQSSFALKTKETXLSKXPXXLXXTLKQFIDGRDKXXEXCFATXLVXAKNFMXXXXXXEXFFEGXXSVXXDXXQNPNYXYKQXSDXSXEDXXXXXXXXXXXXXXXXNRFFNLXNAGXHXXPLADLXXXXXXXXXXXXXXXXXXXXXXXXXXXXXXXXXXXXXXXXXXXXXXXXXXXXXXXXXXXXXXXXXX

SEQ ID NO: 7 - DNA, 60 bp, Artificial Sequence
  Primer A93
ccaatatataataaaatatggaggaatgcgatgctcagaaatacgctaaaatgtgcccaa

SEQ ID NO: 8 - DNA, 60 bp, Artificial Sequence
  Primer A94
tgcctggagatccttactcgagttggatccttatttccatcttaagccatcgttaacttc

SEQ ID NO: 9 - DNA, 37 bp, Artificial Sequence
  Primer A95
ttttactgatgcgtattctttgaattttcaaatagca

SEQ ID NO: 10 - DNA, 34 bp, Artificial Sequence
  Primer A96
tcaaagaatacgcatcagtaaaaaatttgatgga

SEQ ID NO: 11 - DNA, 41 bp, Artificial Sequence
  Primer A97
ttttactgatgtttattctttgaattttcaaatagcaactt

SEQ ID NO: 12 - DNA, 39 bp, Artificial Sequence
  Primer A98
tcaaagaataaacatcagtaaaaaatttgatggacttgg

SEQ ID NO: 13 - DNA, 39 bp, Artificial Sequence
  Primer A99
ttttactgattcgtattctttgaattttcaaatagcaac

SEQ ID NO: 14 - DNA, 36 bp, Artificial Sequence
  Primer A100
tcaaagaatacgaatcagtaaaaaatttgatggact

SEQ ID NO: 15 - DNA, 40 bp, Artificial Sequence
  Primer
  misc_feature (15)..(17): n is a, c, g, or t
aattttttactgatnnntattctttgaattttcaaatagc

SEQ ID NO: 16 - DNA, 40 bp, Artificial Sequence
  Primer
  misc_feature (13)..(15): n is a, c, g, or t
ttcaaagaataannnatcagtaaaaaatttgatggacttg

SEQ ID NO: 17 - DNA, 55 bp, Artificial Sequence
  Primer A120
ccaatatataataaaatatggaggaatgcgatgtctacaacacataacgtccctc

SEQ ID NO: 18 - DNA, 53 bp, Artificial Sequence
  Primer A121
tgcctggagatccttactcgagttggatccttactcaacaggtaaggcgcgag

SEQ ID NO: 19 - DNA, 61 bp, Artificial Sequence
  Primer Y1-11_A13-R
ctaattacatgactcgaggtcgacggtatcgttatttccatcttaagccatcgttaacttc

SEQ ID NO: 20 - DNA, 67 bp, Artificial Sequence
  Primer Y1-11_A13-F
cattagaaagaaagcatagcaatctaatctaagtttaaaacaatgactactcaaccccagctaaatg

SEQ ID NO: 21 - DNA, 60 bp, Artificial Sequence
  Primer YO012
gaaagcatagcaatctaatctaagtttaaaacaatgaccgaacaagtcttattctcagta

SEQ ID NO: 22 - DNA, 59 bp, Artificial Sequence
  Primer YO013
ctaattacatgactcgaggtcgacggtatcgttaagcgttcaacaaattgaaaaatctg

SEQ ID NO: 23 - DNA, 58 bp, Artificial Sequence
  Primer YO014
gaaagcatagcaatctaatctaagtttaaaacaatgaccgaacatgtattattctcag

SEQ ID NO: 24 - DNA, 57 bp, Artificial Sequence
  Primer YO015
ctaattacatgactcgaggtcgacggtatcgttaagcgtttaacaaattgaaaaatc

SEQ ID NO: 25 - DNA, 58 bp, Artificial Sequence
  Primer YO016
gaaagcatagcaatctaatctaagtttaaaacaatgactgaacacgtcttgttctctg

SEQ ID NO: 26 - DNA, 59 bp, Artificial Sequence
  Primer YO017
ctaattacatgactcgaggtcgacggtatcgttaagcgtttaacaaattgaaaaatctg

SEQ ID NO: 27 - DNA, 57 bp, Artificial Sequence
  Primer YO018
gaaagcatagcaatctaatctaagtttaaaacaatgagaagatacatcagaggtggt

SEQ ID NO: 28 - DNA, 57 bp, Artificial Sequence
  Primer YO019
ctaattacatgactcgaggtcgacggtatcgttatgcagcgttcaacaaattgaaaa

SEQ ID NO: 29 - DNA, 58 bp, Artificial Sequence
  Primer YO020
gaaagcatagcaatctaatctaagtttaaaacaatgaccgaacaagtcttattctcag

SEQ ID NO: 30 - DNA, 58 bp, Artificial Sequence
  Primer YO021
ctaattacatgactcgaggtcgacggtatcgttaagcgttcaacaaattgaaaaatct

SEQ ID NO: 31 - DNA, 60 bp, Artificial Sequence
  Primer YO024
gaaagcatagcaatctaatctaagtttaaaacaatgaacttacaatttgaagaaagacca

SEQ ID NO: 32 - DNA, 57 bp, Artificial Sequence
  Primer YO025
ctaattacatgactcgaggtcgacggtatcgttacaaatcagctaaagggtgttcac

SEQ ID NO: 33 - DNA, 59 bp, Artificial Sequence
  Primer YO026
gaaagcatagcaatctaatctaagtttaaaacaatgaacttacactttgaagaattgac

SEQ ID NO: 34 - DNA, 57 bp, Artificial Sequence
  Primer YO027
ctaattacatgactcgaggtcgacggtatcgttagtagtcagacaaatctgctaaag

SEQ ID NO: 35 - DNA, 59 bp, Artificial Sequence
  Primer YO028
gaaagcatagcaatctaatctaagtttaaaacaatgacaatccactgtgaagtattaac

SEQ ID NO: 36 - DNA, 55 bp, Artificial Sequence
  Primer YO029
ctaattacatgactcgaggtcgacggtatcgttaaccaacgtcagccaaagggtg

SEQ ID NO: 37 - DNA, 58 bp, Artificial Sequence
  Primer YO030
gaaagcatagcaatctaatctaagtttaaaacaatgaatgtcacctttgaagaaagag

SEQ ID NO: 38 - DNA, 55 bp, Artificial Sequence
  Primer YO031
ctaattacatgactcgaggtcgacggtatcgttatgccaaatcagctaaagggtg

SEQ ID NO: 39 - DNA, 26 bp, Artificial Sequence
  Primer F1
atgacgcaatttgcatttgtgttccc

SEQ ID NO: 40 - DNA, 19 bp, Artificial Sequence
  Primer F2
ttaaagctcgagcgccgct

(19) United States
(12) Patent Application Publication
(10) Pub. No.: US 2025/0320532 A1
(43) Pub. Date: Oct. 16, 2025

(54) PREPARING DIESTERS OF MALONIC ACID

(71) Applicant: Lygos, Inc., Hayward, CA (US)

(72) Inventors: Nicholas Ohler, Hayward, CA (US); Chi Le, Hayward, CA (US); Daniel Blackburn, Hayward, CA (US); Owen Budavich, Hayward, CA (US); Drew Ross, Hayward, CA (US); Johan Van Walsem, Hayward, CA (US); Jeffrey Dietrich, Hayward, CA (US)

(21) Appl. No.: 18/570,765

(22) PCT Filed: Jun. 10, 2022
(86) PCT No.: PCT/US2022/033057

§ 371 Date: Dec. 15, 2023

Related U.S. Application Data
(60) Provisional application No. 63/212,027, filed on Jun. 17, 2021.

(51) Int. Cl. C12P 7/62; C07C 51/493; C07C 67/08; C07C 67/54; C07C 67/58

(52) U.S. Cl. CPC C12P 7/62; C07C 51/493; C07C 67/08; C07C 67/54; C07C 67/58

(57) ABSTRACT

Methods for the preparation and isolation of malonic acid, a salt or a diesters thereof, preferably bio-based versions of the foregoing are provided.

### CROSS-REFERENCE TO RELATED APPLICATION

This application is a National Stage Application of International Application No. PCT/US2022/033057, filed Jun. 10, 2022, which claims the benefit of priority under 35 U.S.C. 119 (c) and Article 2 of the Paris Convention for the Protection of Industrial Property (1883) to U.S. Provisional Application No. 63/212,027, filed Jun. 17, 2021, the entire contents of which are expressly incorporated herein by reference.

### REFERENCE TO SEQUENCE LISTING

This application contains a Sequence Listing submitted via EFS-web in computer readable form and is hereby incorporated by reference in its entirety. The ASCII copy, created on Jun. 16, 2020, is named LYGOS_0025_02_US_ST25 and is 71 KB in size.

### FIELD

Embodiments disclosed herein relate to methods for extracting and purifying malonic acid diesters such as bio-based malonic acid diesters such as malonate and the compounds and compositions derived from such methods.

### BACKGROUND

Biological systems for producing bio-based malonate via biological fermentation and isolating malonate have been recently described (see U.S. Pat. No. 9,816,114, and PCT App. Pub. Nos. 2018/089,971 and 2019/040,737, and PCT App. No. PCT US2019/017657). However, fermentative production of malonate creates new challenges for extracting and purifying the bio-based malonate produced, and for efficiently integrating extraction and purification processes into the overall production flow. There also remains a need for extracting and purifying downstream products including diester derivatives of malonic acid.

### SUMMARY

In various aspect, the present disclosure provides techniques for methods for the purification of biologically produced malonate, and high-yield production of diester derivatives of malonic acid (for example, dimethyl malonate). In certain embodiments, the malonic acid is obtained from recombinant host cells, and from a biological production of malonic acid. Further, this disclosure provides methods for the biological production of a valuable byproduct, ammonium sulfate, from malonate. The disclosed methods increase process efficiency through the use of one or more of aqueous malonic acid salts, ultrafiltration (UF) or nanofiltration (NF), reactive extraction, and thermal decomposition.

In one aspect, provided herein is a method comprising:
contacting an aqueous solution or aqueous mixture of a malonic acid salt, wherein the aqueous solution or the aqueous mixture of the malonic acid salt has a pH of about 2-about 9, or about 4-about 6, with:
   a lower alkanol and
   an acid
under conditions suitable to provide:
   a diester of malonic acid and the lower alkanol, and optionally,
   a monoester of malonic acid and the lower alkanol, and extracting the diester into an organic solvent, preferentially over the monoester, if present, to provide the diester.

In another aspect, provided herein is a method for isolating a diester of malonic acid from a fermentation broth comprising a fermentation medium and a biomass, the method comprising:
separating the fermentation medium from the biomass by centrifugation;
filtering the fermentation medium via ultrafiltration or nanofiltration;
concentrating the filtered fermentation medium;
esterifying malonic acid present in the concentrated fermentation medium;
extracting mono and dialkyl ester of malonic acid from the concentrated fermentation medium via reactive extraction;
esterifying the extracted monoalkyl ester of malonic acid to the diester of malonic acid via polishing esterification; and
isolating the diester of malonic acid.

In another aspect, provided herein is a method for preparing a diester of malonic acid from a fermentation broth comprising a fermentatrion medium and a biomass, the method comprising:
filtering a fermentation medium of a fermentation broth via ultrafiltration or nanofiltration, wherein the fermentation broth comprises a malonic acid salt;
converting the malonic acid salt into mono and diesters of malonic acid;
extracting into an organic solvent the diester of malonic acid preferentially over the mono ester of malonic acid via reactive extraction;
further converting the monoester of malonic acid present in the organic solvent to the diester of malonic acid by polishing esterfication.

In another aspect, provided herein is a method for preparing a diester of malonic acid from a fermentation broth, comprising:
filtering a fermentation medium of a fermentation broth via ultrafiltration or nanofiltration, wherein the fermentation broth comprises a malonic acid salt;
converting the malonic acid salt into diester and optionally into monoester of malonic acid;
extracting into an organic solvent the diester of malonic acid preferentially over the monoester of malonic acid via reactive extraction;
washing the resulting organic phase with an alkaline aqueous solution to back-extract ionized monoester into the aqueous phase;
recycling the resulting aqueous phase to the salt conversion reactor; and
distilling the resulting organic phase to separate alcohol, solvent, and impurities from the diester of malonic acid.

FIG. 13 provides a schematic of a non-limiting example of an esterification method provided herein comprising reactive extraction where a separate organic solvent, toluene, is used for extraction. This method can be modified based on the disclosure of esterification conditions, reagents, organic solvents, acid catalysts, and such other variables provided herein.

In another aspect, provided herein is a method for preparing a diester of malonic acid from a fermentation broth, comprising:
filtering a fermentation medium from a fermentation broth via ultrafiltration or nanofiltration, wherein the fermentation broth comprises a malonic acid salt;
optionally, concentrating the salt by evaporation;
acidifying the salt using an acidic resin, which is periodically regenerated by washing with acid;
optionally, concentrating the aqueous malonic acid by evaporation;
esterifying the resulting aqueous malonic acid by contacting with an alcohol such as a lower alkalnol via a series of at least two esterification reactors containing an acidic resin, with water removed after each esterification stage and the alcohol added before each esterification stage; and
distilling the resulting organic phase to separate alcohol, solvent, and impurities from the diester of malonic acid.

FIG. 14 provides a schematic of a non-limiting example of an esterification method provided herein comprising reactive extraction where no separate organic solvent is used for extraction. This method can be modified based on the disclosure of esterification conditions, reagents, acid catalysts, and such other variables provided herein.

As used herein, a malonic acid salt includes singly ionized and doubly ionized salts of malonic acid.

As used herein, an organic solvent refers to a water insoluble, water immiscible, substantially water insoluble, or a substantially water immiscible solvent, such as, without limitation aromatic solvents such as toluene or xylenes, ketones such as methyl amyl ketone or methyl isoamyl ketone, ethers such as anisole, and esters such as methyl caprate, dialkyl adipate, alkyl soyate, etc. The term “organic solvents” may also be used to refer to a mixture of organic solvents.

Without being bound by theory, the method of esterifying a salt of malonic acid present in an aqueous solution or a mixture, selectively or preferentially extracting the diester formed into an organic solvent over the monoester, and driving the equilibrium towards diesterification is referred to as reactive extraction. See, e.g., U.S. Pat. Nos. 4,082,788 and 9,233,906, each of which is incorporated herein by reference.

In one embodiment, the malonic acid salt comprises as cations: ammonium, primary ammonium, secondary ammonium, tertiary ammonium, quaternary ammonium, an alkali metal cation, an alkaline earth metal cation, or a mixture thereof.

In another embodiment, the malonic acid salt comprises ammonium cations and malonate anions.

In another embodiment, the acid is a mineral acid. Non limiting examples of mineral acids include, sulfuric acid, hydrochloric acid or another hydrohalic acid, nitric acid, para-toluene sulfonic acid, etc. In another embodiment, the acid is sulfuric acid. In another embodiment, the acid is an acid resin. Non limiting examples of mineral acids include, polystyrene sulfonic acid resins.

In another embodiment, the lower alkanol is a C₁-C₆ alkanol. In another embodiment, the lower alkanol is a C₁-C₃ alkanol. In another embodiment the lower alkanol is methyl alcohol. In another embodiment, the lower alkanol is ethyl alcohol.

In another embodiment, the esterification is performed at a temperature of about 30° C.-about 150° C., or greater than about 70° C. and less than about 100° C.

In another embodiment, the method utilizes an aqueous solution of the malonic acid salt. In another embodiment, the method utilizes an aqueous mixture of the malonic acid salt. In another embodiment, the aqueous solution or the aqueous mixture is obtained from a fermentation broth.

In another embodiment, the aqueous solution is obtained by ultrafiltration of a fermentation medium comprising a malonic acid producing organism, such as Pichia kudriavzevii. In another embodiment, the aqueous solution is obtained by nanofiltration of a fermentation medium comprising Pichia kudriavzevii. As used herein, a fermentation broth comprises a fermentation medium and biomass. The fermentation medium can be separated from the biomass by centrifugation such as ultracentrifugation. It was observed that, surprisingly, ammonium salt of malonic acid passes through nanofilter pores. Accordingly, it is not required to acidify ammonium malonate prior to nanofiltration.

In another embodiment, the method further comprises esterifying extracted malonic acid monoester to the corresponding malonic acid diester,

In one embodiment, the fermentation broth comprises an aqueous ammonium malonate.

In another embodiment, the centrifugation is carried out in two centrifugation steps.

In another embodiment, the ultrafiltration comprises a membrane having a nominal molecular weight cutoff <500,000 Da.

In another embodiment, the reactive extraction uses a countercurrent extraction column, such as a Scheibel column, a Karr column, or a column packed with a stationary solid phase

In another embodiment, the reactive extraction utilizes an organic solvent into which the mono and diesters of malonic acid are extracted. In another embodiment, the mono or diester is a lower alkanol ester.

In another embodiment, the organic solvent comprises toluene, xylenes, o-xylene, anisole, a ketone, or an ester of a carboxylic acid such as an alkyl alkanoate ester.

In another embodiment, the reactive extraction comprises a lower alkanol such as methanol and an acid such as sulfuric acid. In another embodiment, the malonate salt is mixed with the acid to protonate it and release heat of acidification before the aqueous material is contacted with extraction solvent. In another embodiment, the alcohol is mixed with the acidified malonate to perform a significant portion of the esterification before the material is contacted with extraction solvent.

In another embodiment, the reactive extraction, e.g. and without limitation, the reactive extraction performed with acidic resins comprising a simulated moving bed process, excludes an organic solvent for extraction.

In another embodiment, the temperature of the reactive extraction is greater than about 70° C. and less than about 100° C.

In another embodiment, the fermentation broth comprises a malonic acid producing microorganism.

In another embodiment, the microorganism is a yeast selected from Saccharomyces and Pichia.

In another embodiment, the microorganism is Pichia kudriavzevii.

In another aspect, provided herein is a composition comprising more than about 95%, more than about 98%, or more than about 99% of malonic acid or a salt thereof and the rest totaling up to 100% of one or more of lower alkyl levulinate, dialkyl 2-methylmalonic acid ester, monoalkyl malonamide (H₂N(O)CCH₂C(O)O—R), and dialkyl succinate. Suitable alkyl esters include methyl, ethyl, and such other esters. In one embodiment, the composition is free of one ore both of chloroacetic acid or an ester thereof and cyanoacetic acid or an ester thereof. In another embodiment, the malonic acid utilized has a 14C content of substantially greater than zero, since the carbon derives from a terrestrial source (glucose) rather than a subterranean petroleum source.

In some embodiments, the three carbons of the malonic acid (HO₂C—CH₂—CO₂H) utilized herein together has a ¹⁴C content of greater than 0.9 parts per trillion. In some embodiments, the malonic acid utilized or esterified herein has percent modern carbon greater than 75%, or greater than 95%, or is 100%, when measured using ¹⁴C radioisotope analysis corrected with standard methods such as delta ¹³C correction to correct for isotopic fractionation in the natural environment. The percent modern carbon of these three carbon atoms may be measured directly by hydrolyzing the esters completely and separating the resulting malonic acid from the alcohols prior to measurement (e.g. by crystallization), or the percent modern carbon of these three carbon atoms may be measured indirectly by measuring the percent modern carbon of the ester, if the percent modern carbon of the alcohol component of the ester is known, and thereafter calculating the inferred percent modern carbon of the three carbons of the malonic acid core. For example, and without limitation, if a sample of dimethyl malonate is produced using methanol that contains 0% modern carbon, and it is measured as containing 60% modern carbon, then the three carbons of the malonic acid core of the ester must contain 100% modern carbon.

In another embodiment, the composition is a solution. In another embodiment, the composition is a mixture. In another embodiment, the composition is a solid. In another embodiment, the method further comprises converting (e.g., by hydrolysis) the dialkyl ester of malonic acid to malonic acid or a salt thereof of >90%, >95%, or >99% purity.

In certain embodiments, provided herein are methods for the preparation of compositions comprising diester derivatives of malonic acid from malonate produced by a microorganism, such as an engineered microorganism, for example, derived from a renewable carbon source. In certain embodiments, these methods comprise extracting and purifying bio-based malonate and compositions of diester derivatives of malonic acid from fermentation broth. Such methods may comprise the steps of centrifugation, washing, ultrafiltration, evaporation, reactive extraction, solvent stripping, polishing esterification, and/or fractional distillation.

### BRIEF DESCRIPTION OF THE FIGURES

FIG. 1 provides a schematic of the conversion of malonyl-CoA to malonate, as catalyzed by a malonyl-CoA hydrolase (EC 3.1.2.X), according to embodiments of the present disclosure.

FIG. 2 provides a schematic of fermentation and the processing of fermentation broth in accordance with certain embodiments of this disclosure.

FIG. 3 provides a schematic of the synthetic conversion of malonate feedstock to dimethyl malonate, methyl acetate, ammonium sulfate, and methyl pyruvate in accordance with certain embodiments of this disclosure.

FIG. 4 provides an Arrhenius plot to estimate activation energies of esterification reactions in accordance with certain embodiments of this disclosure.

FIG. 5 provides the effluent versus the number of bed volumes for a polishing esterification reaction in accordance with certain embodiments of this disclosure.

FIG. 6 provides a kinetic model fit to the measured reaction compositions for aqueous esterification according to certain embodiments of the methods of this disclosure, as described in Example 23.

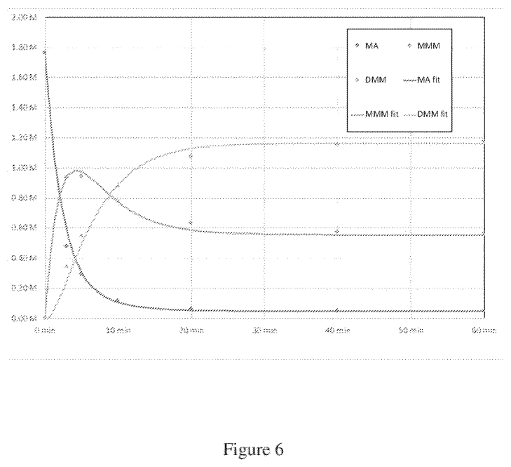

FIG. 7 provides the rate constants vs. H₂SO₄ loading for a 3.06 M malonate feedstock and 9 mole equivalents methanol at 70° C. according to certain embodiments of the methods of this disclosure as described in Example 23.

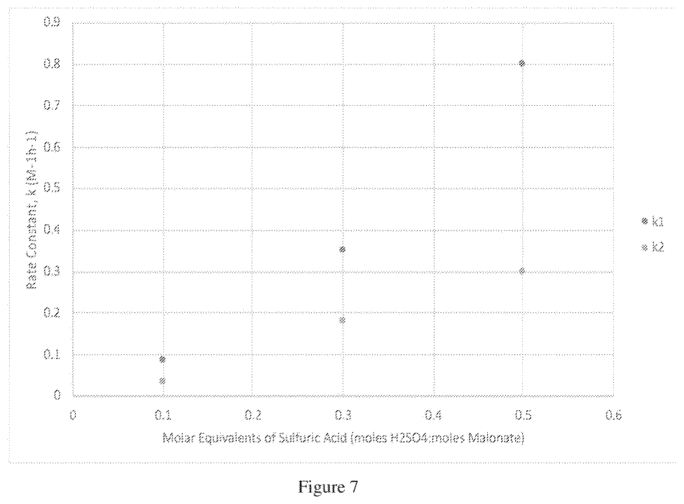

FIG. 8 provides FIG. 9 provides a 1^{st} Order Kinetic Decomposition Plot for malonate in a model raffinate solution in accordance with certain embodiments of this disclosure, as described in Example 25.

FIG. 9A provides images of layer separation for synthetic samples of malonate (Vials 1-2), or reaction intermediates from fermentation broth (Vials 3-5) either containing anti-foaming agents (Vials 2, 4&5) or without anti-foaming agents (Vials 1&3) according to certain embodiments of the methods of this disclosure. FIG. 9B provides images of layer separation for reaction intermediates after overnight resting (Vials 3-5) and ultrafiltration (Vials 7&8), according to certain embodiments of the methods of this disclosure as described in Example 23.

FIG. 10A provides decomposition temperature versus malonate concentration in a model raffinate solution over a five-minute period, in accordance with certain embodiments of this disclosure, as described in Example 24. FIG. 10B provides decomposition temperature versus malonate concentration in a model raffinate solution over a 15-minute period, in accordance with certain embodiments of this disclosure, as described in Example 24. FIG. 10C provides decomposition temperature versus malonate concentration in a model raffinate solution over a 30-minute period, in accordance with certain embodiments of this disclosure, as described in Example 25.

FIG. 11 provides a model of final malonate concentration versus time in a thermal decomposition reaction in a model raffinate solution in accordance with certain embodiments of this disclosure, as described in Example 25. FIG. 11 shows thermal decomposition for the temperature range of 150° C.-200° C.

FIG. 12 provides a model of final malonate concentration versus time in a thermal decomposition reaction in a model raffinate solution in accordance with certain embodiments of this disclosure, as described in Example 25. FIG. 12 shows thermal decomposition for the temperature range of 210° C.-230° C.

FIG. 13 provides a schematic of a non-limiting example of an esterification method provided herein comprising reactive extraction where a separate organic solvent, toluene, is used for extraction.

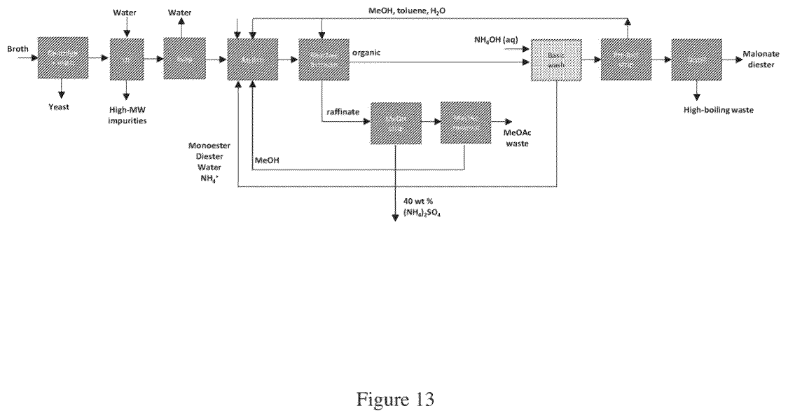

FIG. 14 provides a schematic of a non-limiting example of an esterification method provided herein comprising reactive extraction where no separate organic solvent is used for extraction.

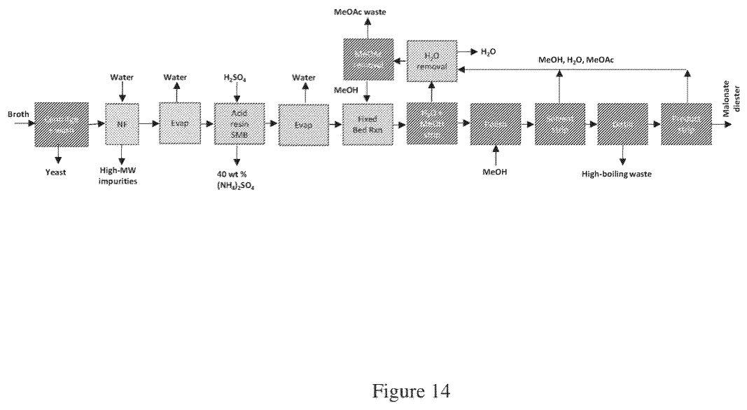

### DETAILED DESCRIPTION

In the following sections, various bio-based malonic acid (“MA”) and ammonium sulfate (“AMS”) compositions and methods for extracting, purifying, and producing these bio-based compositions are described. It is recognized by one skilled in the art that practicing the various embodiments does not require the employment of all or even some of the specific details outlined herein, but rather that concentrations, times and other specific details may be modified through routine experimentation. In some cases, well known methods or components have not been included in the description.

The present disclosure provides recombinant host cells, materials, and methods for the biological production of malonate, purification of biologically produced malonate, and the synthetic conversion of malonate to industrially important chemicals including dimethyl malonate (“DMM”) and AMS. In some embodiments, these methods comprise the removal of impurities which have been discovered to adversely affect the quality of bio-based malonic acid-derived compositions, including DMM and AMS.

In some embodiments, the present disclosure provides a method for isolating diesters of malonic acid from fermentation broth. In some embodiments, the method begins with the fermentation of recombinant host cells suitable for the biosynthetic production of malonate, resulting in fermentation broth containing aqueous ammonium malonate. In some embodiments, the method comprises separating the fermentation broth into fermentation medium and biomass, by centrifugation. In some embodiments, the method further comprises filtering the resultant fermentation medium via ultrafiltration to remove contaminants and further comprises concentrating the fermentation medium via evaporation. In some embodiments, the method further comprises subjecting the fermentation medium (termed herein “malonate feedstock”) to reactive extraction. In some embodiments, reactive extraction yields two products: an organic extract and raffinate. In some embodiments, the organic extract is further stripped and polished, to remove remaining trace contaminants. In some embodiments, the organic extract is distilled via fractional distillation to remove high-boiling impurities, and results in substantially pure diester derivatives of malonic acid, including DMM. In some embodiments, the raffinate is subjected to thermal decomposition to remove residual malonate. In some embodiments, ammonium hydroxide is added to the post-decomposition raffinate to neutralize the raffinate. In some embodiments, the raffinate is stripped and AMS is purified from the raffinate. In some embodiments, a final step of solvent purification results in methyl acetate and methyl pyruvate from the raffinate.

In addition to the overall benefit of biological methods for production of chemicals from renewable feedstock, the specific advantages of the methods provided herein include but are not limited to the elimination of hazardous raw materials that are used for production of petroleum-derived malonic acid and diester derivatives of malonic acid (for example, cyanide, and chloroacetic acid), and the elimination of contaminants present in other bio-based or petroleum-derived malonic acid and diester derivatives of malonic acid (for example, cyanoacetate and sodium cyanide), that can affect industrially useful characteristics of the final product such as curing speed, hardness, odor and color.

Benefits of producing the diesters by the methods disclosed herein include a) case of separation through distillation, b) higher thermal stability during processing with higher yield, c) lower capital and operating costs, and/or d) higher purity. Petrochemically derived malonates contain difficult-to-remove chlorinated intermediates and cyanoacetate impurities. The methods comprise a reactive extraction of a soluble malonate fermentation intermediate, which can significantly lower cost while achieving high purity versus the classical approach of recovering the pure diacid and then esterifying. The methods further comprise ultrafiltration, which can enable reactive extraction of malonates. The methods further comprise the benefit of the production of a valuable byproduct, AMS, from raffinate. These methods also comprise thermal decomposition, to limit malonate content in the AMS byproduct.

In another aspect, this disclosure provides methods for producing malonate in a recombinant host cell, which methods generally comprise culturing the recombinant host cell in fermentation broth under conditions that enable it to produce malonate. In some embodiments, the host cell has been engineered to express more of, or less of, an endogenous enzyme that results in the production of more malonate than a corresponding cell that has not been so engineered. In some embodiments, the methods comprise culturing a recombinant host cell expressing a heterologous enzyme that results in the increased production of malonate. In some embodiments, the host cell used in the methods comprises one or expression vectors comprising encoding heterologous malonyl-CoA hydrolase enzymes. In some embodiments, the fermentation broth is supplemented with carbon sources promoting malonate production and selected from the group consisting of carbon dioxide, ethanol, methanol (“MeOH”), glycerol, acetate, and/or fatty acids.

This disclosure provides methods for purifying malonate from the fermentation broth of a host cell producing malonate, the methods generally comprising culturing a host cell in fermentation broth under conditions that enable the host cell to produce malonate and purifying the malonate from the fermentation broth. In some embodiments, the concentration of malonate in the broth is increased by concentrating the fermentation broth during the purification process. In various embodiments, the concentrating is achieved by reverse osmosis processing, centrifugation, evaporation, including vacuum and heat, “high pass” membrane dewatering, ultrafiltration, nanofiltration, and/or thin film evaporation, or a combination of one or more. In various embodiments, the purification is achieved by adding one or more of the following: a divalent cation, a monovalent cation, ammonium, a monosubstituted amine, a disubstituted amine, a trisubstituted amine, a cationic purification resin, or an acid. In various embodiments, these agents are added in conjunction with one or more organic solvents. In some embodiments, a hydrophobic solvent is used in a liquid-liquid extraction of the fermentation broth. In other embodiments, malonate is purified from the fermentation broth by reactive extraction or distillation with an acid catalyst and an alcohol.

In another aspect, this disclosure provides methods of making compounds derived from malonate and compounds produced by such methods. The methods generally comprise reacting malonate with one or more substrates to produce a compound. In some embodiments, chemicals with established synthetic routes from malonate are produced using biologically derived malonate. In other embodiments, new synthetic routes for the production of useful chemicals are provided that are suitable for use with either a synthetically or biologically derived malonate. In some embodiments, monoalkyl malonate esters are synthesized from biologically derived malonate. In other embodiments, dialkyl malonate esters are synthesized from biologically derived malonate. In some embodiments, an acrylate is synthesized from malonate or malonic acid. In other embodiments, an acrylate is synthesized from malonate monoesters or diesters. In other embodiments, dicarboxylic acids are produced from malonate. Illustrative dicarboxylic acids that can be produced in accordance with the methods of this disclosure include pentanedioic acid, hexanedioic acid, heptanedioic acid, octanedioic acid, nonanedioic acid, decanedioic acid, undecanedioic acid, dodecanedioic acid, the corresponding monoalkyl and dialkyl esters of each and combinations of any of the foregoing. In other embodiments of this disclosure, dicarboxylic acids are produced from a malonate-derived compound. In other embodiments of this disclosure, ε-caprolactam is produced from malonate. In other embodiments of this disclosure, 8-valerolactam is produced from malonate.

While the present disclosure is described herein with reference to aspects and specific embodiments thereof, those skilled in the art will recognize that various changes may be made, and equivalents may be substituted, without departing from this disclosure. The present disclosure is not limited to particular nucleic acids, expression vectors, enzymes, host microorganisms, or processes, as such may vary. The terminology used herein is for purposes of describing particular aspects and embodiments only and is not to be construed as limiting. In addition, many modifications may be made to adapt a particular situation, material, composition of matter, process, process step or steps, in accordance with this disclosure. All such modifications are within the scope of the claims appended hereto.

### Definitions

Unless defined otherwise, all technical and scientific terms used herein have the same meaning as is commonly understood by one of ordinary skill in the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols in Molecular Biology (F. M. Ausubel, et al. eds., (1987)); the series Methods in Enzymology (Academic Press, Inc.): PCR 2: A Practical Approach (M. J. MacPherson, B. D. Flames and G. R. Taylor eds. (1995)), Harlow and Lane, eds. (1988) Antibodies, a Laboratory Manual, and Animal Cell Culture (R. I. Freshney, ed. (1987)).

As used herein, the term “comprising” is intended to mean that the compounds, compositions and processes include the recited elements, but not exclude others. “Consisting essentially of” when used to define compounds, compositions and processes, shall mean excluding other elements of any essential significance to the combination. Thus, a composition consisting essentially of the elements as defined herein would not exclude trace contaminants, e.g., from the isolation and purification method. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%, e.g., by using the prefix, “about.” It is to be understood, although not always explicitly stated that all numerical designations are preceded by the term “about.” It also is to be understood, although not always explicitly stated, that the reagents described herein are merely exemplary and that equivalents of such are known in the art. As used herein, the range, “about x to y” includes about x to about y.

A “salt” is derived from a variety of organic and inorganic counter ions well known in the art and include, when the compound contains an acidic functionality, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate. Salts include acid addition salts formed with inorganic acids or organic acids. Inorganic acids suitable for forming acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Organic acids suitable for forming acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl)benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), glutamic acid, hydroxynaphthoic-acid, salicylic acid, stearic acid, muconic acid, and the like.

Salts also include salts formed when an acidic proton present in the parent compound is either replaced by a metal ion (e.g., an alkali metal ion, an alkaline earth metal ion, or an aluminum ion) or by an ammonium ion (e.g., an ammonium ion derived from an organic base, such as, ethanolamine, diethanolamine, triethanolamine, morpholine, piperidine, dimethylamine, diethylamine, triethylamine, and ammonia).

The terms “a” and “an” and “the” and similar referents as used herein refer to both the singular and the plural, unless otherwise indicated herein or clearly contradicted by context

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂), -n-propyl-(CH₃CH₂CH₂—), isopropyl ((CH₃)₂CH), -n-butyl-(CH₃CH₂CH₂CH₂—), isobutyl ((CH₃)₂CHCH₂—), sec-butyl ((CH₃)(CH₃CH₂)CH), -t-butyl-((CH₃)₃C), -n-pentyl-(CH₃CH₂CH₂CH₂CH₂—), and neopentyl ((CH₃)₃CCH₂—). Lower alkyl refers to C₁-C₆, preferably, C₁-C₃ alkyl.

The term “bio-based” or “renewable” as used herein refers to an organic compound that is synthesized from biologically produced organic components by fermenting a microorganism. For example, diester derivatives of malonic acid or AMS which were synthesized from malonate which was itself synthesized from glucose (for example, derived from cornstarch) by a genetically engineered microorganism is bio-based. Bio-based compounds are distinguished from wholly petroleum-derived compounds or those entirely of fossil origin. A compound of renewable or non-petrochemical origin include carbon atoms that have a non-petrochemical origin. Such non-petrochemical (or bio based or renewable) compounds have a 14C amount substantially higher than zero, such as about 1 parts per trillion or more, because they are derived from photosynthesis based starting material, such as, for example, glucose or another feedstock used in producing such a compound.

As used herein, the term “express”, when used in connection with a nucleic acid encoding an enzyme or an enzyme itself in a cell, means that the enzyme, which may be an endogenous or exogenous (heterologous) enzyme, is produced in the cell. The term “overexpress”, in these contexts, means that the enzyme is produced at a higher level, i.e., enzyme levels are increased, as compared to the wild-type, in the case of an endogenous enzyme. Overexpression of an enzyme can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the enzyme, and the like.

The terms “expression vector” or “vector” refer to a nucleic acid and/or a composition comprising a nucleic acid that can be introduced into a host cell, for example, by transduction, transformation, or infection, such that the cell then produces (“expresses”) nucleic acids and/or proteins other than those native to the cell, or in a manner not native to the cell, that are contained in or encoded by the nucleic acid so introduced. Thus, an “expression vector” contains nucleic acids (ordinarily DNA) to be expressed by the host cell. Optionally, the expression vector can be contained in materials to aid in achieving entry of the nucleic acid into the host cell, such as the materials associated with a virus, liposome, protein coating, or the like. Expression vectors suitable for use in various aspects and embodiments of the present disclosure include those into which a nucleic acid sequence can be, or has been, inserted, along with any operational elements. Thus, an expression vector can be transferred into a host cell and, typically, replicated therein (although, one can also employ, in some embodiments, non-replicable vectors that provide for “transient” expression). In some embodiments, an expression vector that integrates into chromosomal, mitochondrial, or plastid DNA is employed. In other embodiments, an expression vector that replicates extrachromosomally is employed. Typical expression vectors include plasmids, and expression vectors typically contain the operational elements for transcription of a nucleic acid in the vector. Such plasmids, as well as other expression vectors, are described herein or are well known to those of ordinary skill in the art.

The term “heterologous” as used herein refers to a material that is non-native to a cell. For example, a nucleic acid is heterologous to a cell, and so is a “heterologous nucleic acid” with respect to that cell, if at least one of the following is true: (a) the nucleic acid is not naturally found in that cell (that is, it is an “exogenous” nucleic acid); (b) the nucleic acid is naturally found in a given host cell (that is, “endogenous to”), but the nucleic acid or the RNA or protein is produced or present in the host cell in an unnatural (for example, greater or lesser than naturally present) amount; (c) the nucleic acid comprises a nucleotide sequence that encodes a protein endogenous to a host cell but differs in sequence from the endogenous nucleotide sequence that encodes that same protein (having the same or substantially the same amino acid sequence), typically resulting in the protein being produced in a greater amount in the cell, or in the case of an enzyme, producing a mutant version possessing altered (for example higher or lower or different) activity; and/or (d) the nucleic acid comprises two or more nucleotide sequences that are not found in the same relationship to each other in the cell. As another example, a protein is heterologous to a host cell if it is produced by translation of an RNA or the corresponding RNA is produced by transcription of a heterologous nucleic acid; a protein is also heterologous to a host cell if it is a mutated version of an endogenous protein, and the mutation was introduced by genetic engineering.

The terms “host cell”, “host microorganism” and “recombinant host cell” are used interchangeably herein to refer to a living cell that can be (or has been) transformed via insertion of an expression vector. A host microorganism or cell as described herein may be a prokaryotic cell (for example, a microorganism of the kingdom Eubacteria) or a eukaryotic cell. As will be appreciated by one of skill in the art, a prokaryotic cell lacks a membrane-bound nucleus, while a eukaryotic cell has a membrane-bound nucleus.

The terms “isolated” or “pure” refer to material that is substantially, for example greater than 50%, 75%, 90%, 95%, or 99%, free of components that normally accompany it in its native state, for example the state in which it is naturally found or the state in which it exists when it is first produced.

The term “fermentation” or “fermenting” as used herein refers to the feeding of a renewable carbon source (for example, glucose) to a microorganism under conditions that enable the microorganism to consume the carbon source and to produce malonate.

The term “fermentation broth” as used herein refers to a mixture comprising a fermentation medium (liquid; comprising, for example, organic acids, salts, metals, sugars) and biomass (solid; comprising, for example, cells and cell debris).

A carboxylic acid as described herein can be a salt, acid, base, or derivative depending on the structure, pH, and ions present. The terms “malonate” and “malonic acid” are used interchangeably herein unless the context suggests otherwise. Malonic acid is also called propanedioic acid (C₃H₄O₄; CAS #141-82-2).

The term “malonate-derived compounds” as used herein refers to mono-alkyl malonate esters, including, for example and without limitation, mono-methyl malonate (also referred to as monomethyl malonate, CAS #16695-14-0), mono-ethyl malonate (also referred to as monoethyl malonate, CAS #1071-46-1), mono-propyl malonate, mono-butyl malonate, mono-tert-butyl malonate (CAS #40052-13-9), and the like; di-alkyl malonate esters, for example and without limitation, dimethyl malonate (CAS #108-59-8) (“DMM”), diethyl malonate (CAS #105-53-3) (“DEM”), dipropyl malonate (CAS #1117-19-7), dibutyl malonate (CAS #1190-39-2), and the like, and Meldrum's acid (CAS #2033-24-1). The malonate-derived compounds can be produced synthetically from malonate and are themselves valuable compounds but are also useful substrates in the chemical synthesis of a number of other valuable compounds.

As used herein, the term “nucleic acid” and variations thereof shall be generic to polydeoxyribonucleotides (containing 2-deoxy-D-ribose) and to polyribonucleotides (containing D-ribose). “Nucleic acid” can also refer to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. As used herein, the symbols for nucleotides and polynucleotides are those recommended by the IUPAC-IUB Commission of Biochemical Nomenclature (Biochem. 9:4022, 1970). A “nucleic acid” may also be referred to herein with respect to its sequence, the order in which different nucleotides occur in the nucleic acid, as the sequence of nucleotides in a nucleic acid typically defines its biological activity, for example, as in the sequence of a coding region, the nucleic acid in a gene composed of a promoter and coding region, which encodes the product of a gene, which may be an RNA, for example a rRNA, tRNA, or mRNA, or a protein (where a gene encodes a protein, both the mRNA and the protein are “gene products” of that gene).

The term “operably linked” refers to a functional linkage between a nucleic acid expression control sequence (such as a promoter, ribosome-binding site, and transcription terminator) and a second nucleic acid sequence, the coding sequence or coding region, wherein the expression control sequence directs or otherwise regulates transcription and/or translation of the coding sequence.

As used herein, “recombinant” refers to the alteration of genetic material by human intervention. Typically, recombinant refers to the manipulation of DNA or RNA in a cell or virus or expression vector by molecular biology (recombinant DNA technology) methods, including cloning and recombination. Recombinant can also refer to manipulation of DNA or RNA in a cell or virus by random or directed mutagenesis. A “recombinant” cell or nucleic acid can typically be described with reference to how it differs from a naturally occurring counterpart (the “wild-type”). In addition, any reference to a cell or nucleic acid that has been “engineered” or “modified” and variations of those terms, is intended to refer to a recombinant cell or nucleic acid.

As used herein, the term “transcription factor biosensor” refers to a system to detect a substance, for example, malonate, by activating expression of a “marker” or “reporter” gene where reporter gene expression is mediated by a transcription factor that is capable of binding to a promoter and activating transcription upon binding of that substance, for example, malonate. For example, malonate may bind to a transcription factor (for example, MdcY) and activate transcription from a promoter (for example, P_{MdcL}). A “malonate transcription factor” is a transcription factor that, when bound to malonate, can activate a promoter. Thus, MdcY is a malonate transcription factor.

The terms “transduce”, “transform”, “transfect”, and variations thereof as used herein refers to the introduction of one or more nucleic acids into a cell. For practical purposes, the nucleic acid is stably maintained or replicated by the cell for a sufficient period of time to enable the function(s) or product(s) it encodes to be expressed for the cell to be referred to as “transduced”, “transformed”, or “transfected”. Stable maintenance or replication of a nucleic acid may take place either by incorporation of the sequence of nucleic acids into the cellular chromosomal DNA, for example, the genome, as occurs by chromosomal integration, or by replication extrachromosomally, as occurs with a freely-replicating plasmid. A virus can be stably maintained or replicated when it is “infective”: when it transduces a host microorganism, replicates, and (without the benefit of any complementary virus or vector) spreads progeny expression vectors, for example, viruses, of the same type as the original transducing expression vector to other microorganisms, wherein the progeny expression vectors possess the same ability to reproduce.

As used herein, “isolate”, “purify”, and “recover” are used to refer to separation of a substance such as a malonate or an ester thereof from other substances present.

Examples of methods and materials are described below, although methods and materials similar or equivalent to those described herein can also be used in the practice of the present disclosure and will be apparent to those of skill in the art. The materials, methods, and examples are illustrative only and not intended to be limiting.

Wherever a range of values is recited, that range includes every value falling within the range, as if written out explicitly, and further includes the values bounding the range. Thus, a range of “from X to Y” includes every value falling between X and Y, and includes X and Y.

### Recombinant Host Cells

In one aspect, this disclosure provides recombinant host cells suitable for biological production of malonate. Any suitable host cell may be used in practice of the methods of the present disclosure. In some embodiments, the host cell is a recombinant host microorganism in which nucleic acid molecules have been inserted, deleted or modified (i.e., mutated; for example, by insertion, deletion, substitution, and/or inversion of nucleotides), either to produce malonate, or to increase yield, titer, and/or productivity of malonate relative to a “control cell” or “reference cell”. A “control cell” can be used for comparative purposes and is typically a wild-type or recombinant parental cell that does not contain one or more of the modification(s) made to the host cell of interest.

The present disclosure provides recombinant yeast cells suitable for the production of malonate at levels sufficient for subsequent purification and use as described herein, including as a starting material for chemical synthesis of other useful products. In some embodiments, the host cell is a yeast cell. Yeast host cells are excellent host cells for construction of recombinant metabolic pathways comprising heterologous enzymes catalyzing production of small molecule products. There are established molecular biology techniques and nucleic acids encoding genetic elements necessary for construction of yeast expression vectors, including, but not limited to, promoters, origins of replication, antibiotic resistance markers, auxotrophic markers, terminators, and the like. Second, techniques for integration of nucleic acids into the yeast chromosome are well established. Yeast also offers a number of advantages as an industrial fermentation host. Yeast can tolerate high concentrations of organic acids and maintain cell viability at low pH and can grow under both aerobic and anaerobic culture conditions, and there are established fermentation broths and fermentation protocols.

In some embodiments of this disclosure, the recombinant host cell comprising a heterologous nucleic acid encoding a malonyl-CoA hydrolase is a eukaryote. In various embodiments, the eukaryote is a yeast selected from the non-limiting list of genera; Candida, Cryptococcus, Hansenula, Issatchenki, Kluyveromyces, Lipomyces, Pichia, Rhodosporidium, Rhodotorula, Saccharomyces or Yarrowia species. In various embodiments, the yeast is of a species selected from the group consisting of Candida albicans, Candida ethanolica, Candida krusei, Candida methanosorbosa, Candida sonorensis, Candida tropicalis, Cryptococcus curvatus, Hansenula polymorpha, Issatchenki orientalis, Kluyveromyces lactis, Kluyveromyces marxianus, Kluyveromyces thermotolerans, Lipomyces starkeyi, Pichia angusta, Pichia deserticola, Pichia galeiformis, Pichia kodamae, Pichia kudriavzevii, Pichia membranaefaciens, Pichia methanolica, Pichia pastoris, Pichia salictaria, Pichia stipitis, Pichia thermotolerans, Pichia trehalophila, Rhodosporidium toruloides, Rhodotorula glutinis, Rhodotorula graminis, Saccharomyces bayanus, Saccharomyces boulardi, Saccharomyces cerevisiae, Saccharomyces kluyveri, and Yarrowia lipolytica. This list encompasses yeast in the broadest sense, including both oleaginous and non-oleaginous strains.

Alternative recombinant host cells are provided by this disclosure for biological production of malonate. Illustrative examples include eukaryotic, prokaryotic, and archaea cells. Illustrative examples of eukaryotic cells include, but are not limited to: Aspergillus niger, Aspergillus oryzae, Crypthecodinium cohnii, Cunninghamella japonica, Entomophthora coronata, Mortierella alpina, Mucor circinelloides, Neurospora crassa, Pythium ultimum, Schizochytrium limacinum, aureum, Thraustochytrium Trichoderma reesei and Xanthophyllomyces dendrorhous. In general, if a eukaryotic cell is used, a non-pathogenic strain is employed. Illustrative examples of non-pathogenic strains include, but are not limited to: Pichia pastoris and Saccharomyces cerevisiae. In addition, certain strains, including Saccharomyces cerevisiae, have been designated by the United States Food and Drug Administration (“FDA”) as Generally Regarded As Safe (“GRAS”) and so can be conveniently employed in various embodiments of the methods of this disclosure.

In other embodiments, the host cell is a bacterial cell. In various embodiments, the host cell is a bacterial cell. In some embodiments, the bacterial cell is selected from the group consisting of Bacillus, Clostridium, Corynebacterium, Escherichia, Pseudomonas, and Streptomyces. In some embodiments, the host cell is an E. coli cell.

Illustrative examples of recombinant prokaryotic host cells provided by this disclosure include, but are not limited to, Bacillus subtilis, Brevibacterium ammoniagenes, Clostridium beigerinckii, Enterobacter sakazakii, Lactococcus lactis, Mesorhizobium loti, Pseudomonas aeruginosa, Pseudomonas putida, Rhodobacter capsulatus, Rhodobacter sphaeroides, Salmonella enterica, Salmonella typhi, Salmonella typhimurium, Shigella flexneri, Staphylococcus aureus, Streptomyces ambofaciens, Streptomyces aureofaciens, Streptomyces aureus, Streptomyces fungicidicus, Streptomyces griseochromogenes, Streptomyces griseus, Streptomyces lividans, Streptomyces olivogriseus, Streptomyces rameus, Streptomyces tanashiensis, and Streptomyces vinaceus. Certain of these cells, including Bacillus subtilis, Lactobacillus acidophilus, have been designated by the FDA as GRAS and so are employed in various embodiments of the methods of this disclosure.

Escherichia coli (“E. coli”) is an excellent prokaryotic host cell for metabolic pathway construction, and E. coli is also well utilized in industrial fermentation of small-molecule products. Unlike most wild type yeast strains, wild type E. coli can catabolize both pentose and hexose sugars as carbon sources. E. coli also has a shorter doubling time relative to yeast, enabling experiments to be conducted more rapidly. The present disclosure provides a wide variety of E. coli host cells suitable for the production of malonate as described herein. In various embodiments of the methods of this disclosure, the recombinant host cell comprising a heterologous nucleic acid encoding a malonyl-CoA hydrolase is an E. coli cell.

Generally, the recombinant host cells of this disclosure have been genetically modified for improved malonate yield, titer, and/or productivity. In various embodiments, the host cells have been modified for increased malonate biosynthesis through one or more host cell modifications selected from the group consisting of modifications that result in increased acetyl-CoA biosynthesis, increased malonyl-CoA biosynthesis, decreased malonate catabolism increased secretion of malonate into the fermentation broth, increased host cell tolerance to malonate in the fermentation broth, and/or increased host cell catabolism of carbon sources (for example, acetate, alginate, ethanol, fatty acids, lignocellulosic biomass, methanol, pentose sugars, and syn gas).

In various embodiments recombinant host cells provided by the present disclosure can be produced by introduction of one or more of the heterologous (foreign, non-native) nucleic acids provided by this disclosure, which encode a wild-type or mutated form of an acyl-CoA hydrolase, thereby allowing the recombinant host cell to produce malonate. Non-limiting examples of acyl-CoA hydrolases encoded by the nucleic acids provided by this disclosure and suitable for malonyl-CoA hydrolysis include wild-type and modified enzymes selected from the group consisting of 3-hydroxyisobutyryl-CoA hydrolases (EC 3.1.2.4), 3-hydroxypropionyl-CoA hydrolases (EC 3.1.2.4), acetoacetyl-CoA hydrolases (EC 3.1.2.11), methylmalonyl-CoA hydrolases (EC 3.1.2.17), propionyl-CoA hydrolases (EC 3.1.2.18), succinyl-CoA hydrolases (EC 3.1.2.3), and malonyl CoA: ACP transacylases (EC 2.3.1.39) mutated as provided herein to have malonyl CoA hydrolase activity.

### Malonyl-CoA Hydrolase Enzymes

In accordance with one aspect of this disclosure, malonate is produced through the action of a malonyl-CoA hydrolase catalyzing the conversion of malonyl-CoA to malonate. The host cell making the malonyl-CoA hydrolase is a recombinant host cell; in many embodiments, the host cell has been genetically modified to comprise heterologous nucleic acid(s) encoding malonyl-CoA hydrolase enzyme(s) catalyzing hydrolysis of malonyl-CoA to malonate.

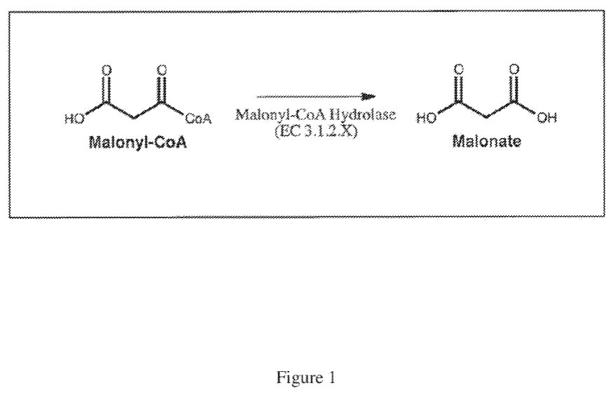

A schematic representation of one of the malonyl-CoA hydrolase pathways provided by this disclosure is shown in FIG. 1. The present disclosure results in part from the discovery that various acyl-CoA hydrolases and transacylases can be engineered to have malonyl-CoA hydrolase activity and so be useful for biological production of malonate. Non-limiting examples of acyl-CoA hydrolases suitable for modification for malonyl-CoA hydrolysis include any of those from the group consisting of 3-hydroxyisobutyryl-CoA hydrolases (EC 3.1.2.4), 3-hydroxypropionyl-CoA hydrolases (EC 3.1.2.4), acetoacetyl-CoA hydrolases (EC 3.1.2.11), methylmalonyl-CoA hydrolases (EC 3.1.2.17), propionyl-CoA hydrolases (EC 3.1.2.18), succinyl-CoA hydrolases (EC 3.1.2.3), and malonyl CoA: ACP transacylases (EC 2.3.1.39) mutated as provided herein to have malonyl CoA hydrolase activity.

In some embodiments, the malonyl-CoA hydrolase used to produce malonate in accordance with this disclosure is a mutated S. cerevisiae EHD3 acyl-CoA hydrolase (see SEQ ID NO: 1 for the wild-type EHD3 amino acid sequence). One such mutant with altered substrate specificity is the E124V mutant (see Rouhier, “Characterization of YDR036C from Saccharomyces cerevisiae.” Dissertation, Miami University, Miami University and OhioLINK (2011)). In other embodiments of this disclosure, a yeast cell expressing the E124V mutant is used to produce malonate in accordance with this disclosure. In yet another embodiment, an oleaginous yeast cell expressing the E124V mutant is used to produce malonate in accordance with this disclosure.

Amino acids in a protein coding sequence are identified herein by providing the single-letter abbreviation as follows A (alanine), R (arginine), N (asparagine), D (aspartic acid), C (cysteine), Q (glutamine), E (glutamic acid), G (glycine), H (histidine), L (leucine), I (isoleucine), K (lysine), M (methionine), F (phenylalanine), P (proline), S (serine), T (threonine), W (tryptophan), Y (tyrosine), V (valine). Specific amino acids in a protein coding sequence are identified by their single-letter abbreviation followed by the amino acid position in the protein coding sequence where I corresponds to the amino acid (typically methionine) at the N-terminus of the protein. For example, E124 in S. cerevisiae wild type EHD3 refers to the glutamic acid at position 124 from the EHD3 N-terminal methionine (i.e., M1). Amino acid substitutions (i.e., point mutations) are indicated by identifying the mutated (i.e., progeny) amino acid after the single-letter code and number in the parental protein coding sequence; for example, E124A in S. cerevisiae EHD3 refers to substitution of alanine for glutamic acid at position 124 in the EHD3 protein coding sequence. The mutation may also be identified in parentheticals, for example EHD3 (E124A). Multiple point mutations in the protein coding sequence are separated by a backslash (/); for example, EHD3 E124A/E125A indicates that mutations E124A and E125A are both present in the EHD3 protein coding sequence.

The present disclosure provides expression vectors for the E124A mutant that can be used in E. coli host cells, rendering them capable of producing malonate. This is achieved, for example, by employing expression vectors with a lower copy number or weaker promoter than used by Rouhier. Examples of lower copy number expression vectors include, but are not limited to pSC101 origin expression vectors, p15a origin expression vectors, and expression vectors that integrate into the chromosomal DNA. Examples of weaker promoters than the T7 promoter used by Rouhier include, but are not limited to the P_{LacO1}, P_{TRC}, and P_{BAD} promoters. In some embodiments, the vector has a pSC101 origin of replication. In other embodiments, the promoter used for expression of the EHD3 E124A mutant coding sequence is the P_{lacO1} promoter. Additionally, the present disclosure provides vectors for yeast host cells that code for the expression of the E124A mutant. The genetically modified S. cerevisiae EHD3 E124A expression vectors of this disclosure can be used in vivo for the production of malonate in E. coli, S. cerevisiae and P. kudriavzevii, and the methods of this disclosure provide means for the subsequent purification of malonate from fermentation broth of these strains, and the synthetic conversion of malonate into derivative small-molecule compounds.

The present disclosure also provides the E124S mutant of EHD3 for use as a malonyl-CoA hydrolase, vectors for expressing this mutant, and host cells that express this mutant and produce malonate (see Example 1). Wild-type S. cerevisiae EHD3 catalyzes the hydrolysis of 3-hydroxypropionyl-CoA (3HPA-CoA) and 3-hydroxyisobutyryl-CoA (3HIBA-COA) and E124 is predicted to interact with the terminal hydroxyl moiety on 3HPA-CoA, stabilizing the substrate in the EHD3 active site (see Rouhier, supra). Certain aspects of this disclosure arise from the discovery that specific E124 point mutations increase enzyme hydrolysis of malonyl-CoA, producing malonate. Mutation of E124 to a nucleophilic amino acid (for example, S or T), basic amino acid (for example, H, K, or R), or amide amino acid (for example, N or Q) improves the binding of malonyl-CoA in the EHD3 active site over 3-hydroxypropionyl-CoA and increases malonate production (relative to the unmutated counterpart enzyme). The E124S, E124T, E124N, E124Q, E124H, E124K, and E124R mutations also decrease production of byproducts (for example, acetate, propionate, isobutyrate, and succinate) due to decreased hydrolysis of endogenous host cell acyl-CoA molecules. The E124S point mutation places a hydroxyl moiety in a position that promotes hydrogen bonding between the serine residue and the terminal carboxylate group of malonyl-CoA. The E124Q point mutation places the glutamine amide group in a position near the terminal carboxylate group of malonyl-CoA. The E124K point mutation places the lysine amine group in a position that promotes hydrogen bonding between the lysine residue and the terminal carboxylate group of malonyl-CoA. In contrast to the nucleophilic, amide, and basic E124 point mutations described above, mutations E124A and E124V remove the presence of a charged amino acid at position 124; these mutations both eliminate hydrogen bonding between the terminal carboxylate on malonate and the EHD3 124 amino acid sidechain and open the EHD3 active site to promiscuous activity, increasing undesirable byproduct formation and decreasing malonate production.

In some embodiments of this disclosure, an E. coli host cell expressing the E124S mutant is used to produce malonate. In other embodiments of this disclosure, a yeast host cell, for example a Pichia kudriavzevii host cell, expressing the E124S mutant is used to produce malonate. In other embodiments, an oleaginous yeast host cell expressing the E124S mutant is used to produce malonate. In some embodiments of this disclosure, an E. coli host cell expressing the E124Q mutant is used to produce malonate. In other embodiments of this disclosure, a yeast host cell, for example a Pichia kudriavzevii host cell, expressing the E124Q mutant is used to produce malonate. In other embodiments, an oleaginous yeast host cell expressing the E124Q mutant is used to produce malonate. In some embodiments of this disclosure, an E. coli host cell expressing the E124K mutant is used to produce malonate. In other embodiments of this disclosure, a yeast host cell, for example a Pichia kudriavzevii host cell, expressing the E124K mutant is used to produce malonate. In other embodiments, an oleaginous yeast host cell expressing the E124K mutant is used to produce malonate. In some embodiments of this disclosure, an E. coli host cell expressing the E124H mutant is used to produce malonate. In other embodiments of this disclosure, a yeast host cell, for example a Pichia kudriavzevii host cell, expressing the E124H mutant is used to produce malonate. In other embodiments of this disclosure, an oleaginous yeast host cell expressing the E124H mutant is used to produce malonate. In some embodiments of this disclosure, an E. coli host cell expressing the E124R mutant is used to produce malonate. In other embodiments of this disclosure, a yeast host cell, for example a Pichia kudriavzevii host cell, expressing the E124R mutant is used to produce malonate. In other embodiments of this disclosure, an oleaginous yeast host cell expressing the E124R mutant is used to produce malonate. In other embodiments, a recombinant host cell expressing an EHD3 E124 nucleophilic amino acid point mutation (i.e., E124S or E124T) is used to produce malonate. In other embodiments, a recombinant host cell expressing an EHD3 E124 basic amino acid point mutation (i.e., E124H, E124K, or E124R) is used to produce malonate. In other embodiments, a recombinant host cell expressing an EHD3 E124 amide amino acid point mutation (i.e., E124N or E124Q) is used to produce malonate.

The present disclosure also provides a mutated EHD3 comprising a mutated active site, vectors for expressing the mutant, and host cells that express the mutant and produce malonate. Certain aspects of the present disclosure arose, in part, from the discovery that specific amino acids (i.e., F121, and F177) are involved in acyl-CoA substrate binding, and introduction of specific point mutations increase malonyl-CoA hydrolysis and production of malonate. Introduction of mutation F1211 or F121L increases malonyl-CoA access to the active site. Similarly, introduction of mutation F1771 or F177L increases malonyl-CoA access to the active site. One or more point mutations at amino acid positions F121 or F177 can be introduced alone, or along with an E124 point mutation. In various embodiments, a F121 and/or F177 point mutation is introduced along with an E124 point mutation. In some embodiments, a recombinant host cell expressing an EHD3 F1211 or F121L mutant is used to produce malonate. In other embodiments, a recombinant host cell expressing an EHD3 F1771 or F178L mutant is used to produce malonate. In these embodiments, the recombinant host cell can be, without limitation, an E. coli or yeast, including but not limited to S. cerevisiae, P. kudriavzevii or other yeast, host cell.

The present disclosure also provides mutated EHD3 comprising a mutated mitochondrial targeting sequence, vectors for expressing the mutant, and host cells that express the mutant and produce malonate. In an S. cerevisiae host, wild-type EHD3 is localized in the mitochondria. Malonyl-CoA is found in both the mitochondria and the cytosol; EHD3 catalyzed hydrolysis of cytosolic malonyl-CoA requires localization of an EHD3 to the cytosol. Certain aspects of the present disclosure arose from the discovery that mutations of the EHD3 mitochondrial targeting sequence can increase production of malonate. The EHD3 amino acids involved in mitochondrial targeting include R3, K7, K14, K18, and R22, and mutation of one or more of these basic amino acids to a hydrophobic amino acid (i.e., A or V) abrogates mitochondrial targeting. In some embodiments, a recombinant host comprising an EHD3 consisting of one or more mutations to A or V at amino acids selected from the group consisting of R3, K7, K14, K18, and R22 is used to produce malonate. In some embodiments, the recombinant host is a yeast strain. In some embodiments, the recombinant host is S. cerevisiae. In some embodiments, the recombinant host cell is P. kudriavzevii. In still further embodiments, the recombinant host cell contains one or more copies of an EHD3 with the mitochondrial targeting sequence unaltered (i.e., wild-type) and one or more copies of an EHD3 with the mitochondrial targeting sequence mutated.

Thus, in one aspect of this disclosure, the recombinant host cell comprises a heterologous nucleic acid encoding a mutant S. cerevisiae EHD3 that results in increased production of malonate relative to host cells not comprising the mutant EHD3. In some embodiments, the mutant EHD3 is heterologously expressed in E. coli. In other embodiments, the mutant EHD3 is heterologously expressed in S. cerevisiae. In other embodiments, the mutant EHD3 is heterologously expressed in P. kudriavzevii. In other embodiments, the mutant EHD3 is heterologously expressed in an oleaginous yeast cell. In some embodiments, the mutant EHD3 contains a point mutation at position E124. In some embodiments, the point mutation at residue E124 is either E124A or E124V. In some embodiments, the point mutation at E124 is E124S or E124T. In some embodiments, the point mutation at E124 is E124S. In some embodiments, the point mutation at E124 is a basic amino acid selected from the group consisting of E124H, E124K, and E124R. In some embodiments, the point mutation at E124 is E124H. In some embodiments, the point mutation at E124 is E124K. In some embodiments, the point mutation at E124 is E124R. In some embodiments, the point mutation at residue E124 is E124N or E124Q. In some embodiments, the point mutation at residue E124 is E124Q. In some embodiments, one or more EHD3 amino acids selected from the group consisting of F121 and F177 are mutated to I or L. In some embodiments, one or more EHD3 amino acids selected from the group consisting of R3, K7, K14, K18, and R22 are mutated to either A or V.

In another aspect of this disclosure, an enzyme other than, or in addition to, EHD3 is utilized as a malonyl-CoA hydrolase to produce malonate in accordance with this disclosure. In some embodiments, Haemophilus influenzae YciA is heterologously expressed in a host cell to produce malonate in accordance with this disclosure (see Zhuang et al. Biochemistry 47:2789-2796 (2008)). In other embodiments, the malonyl-CoA hydrolase is an acyl-CoA hydrolase endogenous to Rattus norvegicus (see Kovachy et al., J. Biol. Chem. 258:11415-11421 (1983)). In other embodiments, the malonyl-CoA hydrolase is the acyl-CoA hydrolase from brown adipose tissue mitochondrial protein fraction from Mesocricetus auratus (see Alexson et al., J. Biol. Chem. 263:13564-13571 (1988)).

Thus, in accordance with some embodiments of this disclosure, acyl-CoA hydrolases other than, or in addition to, EHD3 (from S. cerevisiae or homologous enzymes from other organisms) can be used for biological synthesis of malonate in a recombinant host. In some embodiments, the recombinant host is S. cerevisiae. In other embodiments, the recombinant host is E. coli. In other embodiments, the recombinant host is a yeast other than S. cerevisiae, for example a Pichia kudriavzevii host cell. In various embodiments, the host is modified to express a mutated enzyme selected from the group consisting of S. albicans EHD3, H. sapiens HIBCH (UniProt: Q6NVY1), A. thaliana CHY1 (UniProt: Q9LKJ1), R. norvegicus HIBCH (UniProt: Q5XIE6), M. musculus HIBCH (UniProt: Q8QZS1), G. gallus HIBCH (UniProt: Q5ZJ60), B. taurus HIBCH (UniProt: Q2HJ73), D. rerio HIBCH (UniProt: Q58EB4), B. cereus Bch, P. aeruginosa Hich, E. coli YciA, H. influenzae YciA, M. musculus ACOT4, M. musculus ACOT8, S. enterica SARI_01218, A. pernix K1, C. hutchinsonii Chut02003666, S. solfataricus P2 SS02287, S. acidocaldarius DSM 639 Saci_0145, P. aerophilum str. IM2 PAE3404, D. melanogaster CG1635, P. carbinolicus DSM 2380 Pcar_1366, A. dehalogenans 2CP-C 110, G. gallus ACOT9, and X. laevis MGC114623.

One or multiple suitably mutated acyl-CoA hydrolases can be used in accordance with this disclosure to convert malonyl-CoA to malonate in a host cell. Moreover, acyl-CoA hydrolases other than those specifically disclosed herein can be utilized in mutated or heterologously expressed form, and other appropriate enzymes can be identified, modified, and expressed to achieve the desired malonyl-CoA hydrolase activity as disclosed herein.

### Consensus Sequences

Malonyl-CoA hydrolases of this disclosure comprise those that are homologous to consensus sequences provided by this disclosure. As noted above, any enzyme substantially homologous to an enzyme specifically described herein can be used in a host cell of this disclosure. One enzyme is homologous to another (the “reference enzyme”) when it exhibits the same activity of interest and can be used for substantially similar purposes. Generally, homologous enzymes share substantial sequence identity. Sets of homologous enzymes generally possess one or more specific amino acids that are conserved across all members of the consensus sequence protein class.

The present disclosure provides consensus sequences useful in identifying and constructing malonyl-CoA hydrolases of this disclosure. In various embodiments, these malonyl-CoA hydrolase consensus sequences contain active site amino acid residues believed to contribute to the formation of an oxyanion hole responsible for stabilizing the enolate anion intermediate derived from a malonyl-CoA substrate as well as the amino acid residues involved with malonyl-CoA binding. A homologous enzyme, relative to a consensus sequence provided by this disclosure, may have different amino acids at non-conserved positions or amino acid(s) inserted or deleted, so long as those differences do not negatively affect or only insignificantly negatively affect the malonyl-CoA hydrolysis activity of interest. Thus, a homologous enzyme has an enzymatic activity that is identical, or essentially identical, or at least substantially similar with respect to ability to hydrolyze malonyl CoA to that of one of the enzymes exemplified herein. A homologous enzyme may be found in nature or be an engineered mutant thereof. A homologous enzyme may be identified or constructed from another enzyme by comparison to a consensus sequence herein; if an enzyme shares substantial homology to a consensus sequence herein but has suboptimal, including no, malonyl-CoA hydrolase activity, then, in accordance with this disclosure, it is mutated to conform to a consensus sequence provided herein to provide a malonyl-CoA hydrolase of this disclosure.

This disclosure provides four malonyl-CoA hydrolase consensus sequences: (i) malonyl-CoA hydrolase based on EHD3 EC 3.1.2.4 (ii) malonyl-CoA hydrolase based on Bacillus EC 3.1.2.4 malonyl-CoA hydrolase, (iii) malonyl-CoA hydrolase based on Pseudomonas EC 3.1.2.4 malonyl-CoA hydrolase, and (iv) malonyl-CoA hydrolase based on from both Bacillus and Pseudomonas EC 3.1.2.4. The consensus sequences provide a sequence of amino acids in which each position identifies the amino acid most likely to be found at a specified position in a malonyl-CoA hydrolase of that class. In the consensus sequences, a dash (−) indicates the presence of a gap that may exist when a homologous enzyme sequence is aligned against the consensus sequence. A plus (+) indicates a position in the consensus sequence where the amino acid is highly non-conserved; a homologous protein may contain one of many different amino acids at these non-conserved positions. One-letter amino acid codes are defined above. At some positions shown in the consensus sequence, the homologous enzyme may contain one of several amino acids, and for these positions, additional one letter codes are as follows: B (amino acid is R, K, or H), J (amino acid is D or E), O (amino acid is I, L, or V), U (amino acid is S or T), and X1 (amino acid is R, H, K, S, T, N, Q, Y).

### EHD3 EC 3.1.2.4 Malonyl-CoA Hydrolase Consensus Sequence

This disclosure provides an EHD3 malonyl-CoA hydrolase consensus sequence (SEQ ID NO:3), and in various embodiments, suitable malonyl-CoA hydrolases for use in the methods of this disclosure have at least 63% identity to this EHD3 malonyl-CoA hydrolase consensus sequence. In various embodiments, the malonyl-CoA hydrolases suitable for use in the methods of this disclosure have 65%, 70%, 80%, 90%, or 95% or more identity to this EHD3 consensus sequence. Proteins having homology to this consensus sequence include UniProt ID: C5DE94 (63% identity), UniProt ID: Q6CJH2 (64% identity), UniProt ID: G2WAE2 (66% identity), UniProt ID: J8Q6P9 (66% identity), UniProt ID: G8COHO (68% identity), UniProt ID: C5DX08 (68% identity), UniProt ID: P28817 (69% identity), UniProt ID: A7TTD5 (69% identity), UniProt ID: J7S9J9 (70% identity), UniProt ID: Q6FM09 (71% identity), UniProt ID: 12H4L2 (71% identity), UniProt ID: H2AME2 (73% identity), UniProt ID: G8ZTJ4 (77% identity), UniProt ID: GOW418 (77% identity), UniProt ID: GOV818 (78% identity), and UniProt ID: J5S5X3 (79% identity). In some embodiments, an EHD3 malonyl-CoA hydrolase with equal to or greater than 63% identity to the consensus sequence SEQ ID NO:3 is expressed in a recombinant host cell and used to produce malonate in accordance with this disclosure.

In mutant and wild-type enzymes homologous to this consensus sequence (SEQ ID NO: 3), amino acids that are highly conserved arc V101, R110, L114, R116, K119, L120, N121, A122, L123, L135, E137, Y138, K140, S141, S151, R156, C159, G161, G162, D163, V164, A168, F185, E188, Y189, S190, N192, A196, T197, K200, M206, G208, 1209, T210, M211, G212, G213, G214, V215, G216, H220, P222, F223, R224, T227, E228, T230, M234, P235, E236, D238, 1239, G240, F242, P243, D244, V245, F249, P252, Q263, Y267, L268, T271, G272, G277, G284, S287, H288, Y289, L298, R301, L302, E304, E333, F334, L352, V354, 1355, F359, L374, F391, L399, K402, S403, S406, N417, D429, L430, T432, A433, E449, F450, K457, L458, K461, W468, L494, T502, Y506, P507, L514, P515, and K561. In various embodiments, malonyl-CoA hydrolase enzymes homologous to this consensus sequence (SEQ ID NO:3) contain at least a plurality of these conserved amino acids, often a majority of these conserved amino acids, and sometimes all of these conserved amino acids.

Some amino acids in this consensus sequence (SEQ ID NO:3) contribute to activity and conserved across all members of the class. Malonyl-CoA hydrolase enzymes in the EHD3 class contain six active site residues involved with hydrolase activity: (i) three active site amino acid residues (G161, G162, G213) in the consensus sequence believed to contribute to the formation of an oxyanion hole responsible for stabilizing the enolate anion intermediate derived from the malonyl-CoA substrate; (ii) two amino acid residues (E236, D244) of the consensus sequence useful for acyl-CoA hydrolysis; and (iii) an amino acid residue at position 188 (of SEQ ID NO: 3) believed to contribute to malonyl-CoA substrate binding. Of these six residues, then, five are present in the consensus sequence (SEQ ID NO:3), and the sixth, at position 188 (amino acid X1 in the consensus) is selected from the group of polar or positively charged amino acids (R, H, K, S, T, N, Q, Y) to provide a malonyl CoA hydrolase of this disclosure capable of producing malonate in a recombinant host cell. The six residues from the consensus sequence (G161, G162, G213, E236, D244, X1188) correspond to G99, G100, G149, E172, D180, and E124 (typically mutated to X1), respectively, in S. cerevisiae EHD3 used to illustrate this disclosure in Example 1.

### Bacillus EC 3.1.2.4 Malonyl-CoA Hydrolase Consensus Sequence

This disclosure provides a Bacillus EC 3.1.2.4 malonyl-CoA hydrolase consensus sequence (SEQ ID NO:4), and in various embodiments, suitable malonyl-CoA hydrolases for use in the methods of this disclosure have at least 86% identity to this Bacillus EC 3.1.2.4 malonyl-CoA hydrolase consensus sequence. In various embodiments, the malonyl-CoA hydrolases suitable for use in the methods of this disclosure have 90%, or 95% or more identity to this Bacillus EC 3.1.2.4 malonyl-CoA hydrolase consensus sequence. Proteins having homology to this consensus sequence include Bacillus EC 3.1.2.4 proteins UniProt ID: C2TX63 (92% identity), UniProt ID: C2UV40 (91% identity), UniProt ID: C2QBT2 (93% identity), UniProt ID: C2XTUO (93% identity), UniProt ID: C2PVQ0 (93% identity), UniProt ID: C3A5N3 (93% identity), UniProt ID: C2SJV4 (93% identity), UniProt ID: C2Z7U1 (92% identity), UniProt ID: C2VTI4 (97% identity), UniProt ID: B3Z9Y3 (97% identity), UniProt ID: B7JNH7 (97% identity), UniProt ID: Q63BK8 (97% identity), UniProt ID: BOQ3Q4 (97% identity), UniProt ID: BOAQX0 (97% identity), UniProt ID: B3YSW2 (97% identity), UniProt ID: C2NHG5 (97% identity), UniProt ID: B3ZIZ8 (97% identity), UniProt ID: C2QSV2 (97% identity), UniProt ID: C3C255 (97% identity), UniProt ID: B5UZZI (96% identity), UniProt ID: C2MKL7 (95% identity), UniProt ID: B9IZZ9 (95% identity), UniProt ID: FOPNG8 (95% identity), UniProt ID: Q738L0 (97% identity), UniProt ID: C2PEV7 (95% identity), UniProt ID: C2YRH7 (96% identity), UniProt ID: Q4MU30 (95% identity), UniProt ID: Q81DR3 (96% identity), UniProt ID: C2W7W8 (89% identity), and UniProt ID: A7GPH6 (86% identity). In various embodiments, a Bacillus EC 3.1.2.4 malonyl-CoA hydrolase with equal to or greater than 86% identity to the consensus sequence SEQ ID NO:4 is expressed in a recombinant host cell and used to produce malonate in accordance with this disclosure.

In mutant and wild type enzymes homologous to this consensus sequence (SEQ ID NO: 4) amino acids that are highly conserved are M1, T2, E3, V5, L6, F7, S8, G13, V14, A15, 117, T18, L19, N20, R21, P22, K23, A24, L25, N26, S27, L28, S29, Y30, M32, L33, 136, G37, K39, L40, K41, E42, W43, E44, 149, 152, V53, L54, K55, G56, A57, G58, K60, G61, F62, C63, A64, G65, G66, D67, 168, K69, T70, L71, Y72, E73, A74, R75, S76, N77, E78, A80, L81, Q82, A84, E85, F87, F88, E90, E91, Y92, 194, D95, T96, Y99, Y101, K103, P104, 1105, 1106, A107, C108, L109, D110, G111, 1112, V113, M114, G115, G116, G117, V118, G119, L120, T121, N122, G123, A124, R127, 1128, V129, T130, T133, K134, W135, A136, M137, P138, E139, M140, N141, 1142, G143, F144, F145, P146, D147, V148, G149, A150, A151, Y152, F153, L154, N155, A157, P158, G159, G162, V165, A166, L167, A169, L172, K173, A174, D176, V177, L178, 1180, A182, A183, D184, L192, F195, L196, W204, V210, L214, K215, L231, E236, H241, F242, E248, 1250, 1251, S253, L254, E255, F261, L269, L270, S271, K272, S273, P274, S276, L277, K278, V279, T280, L281, K282, Q283, G287, K290, S291, E293, C295, F296, A297, T298, D299, L300, L302, A303, K304, N305, F306, M307, R308, H309, D311, F312, F313, E314, G315, V316, R317, S318, V320, D322, K323, D324, Q325, N326, P327, Y329, K330, Y331, D336, V337, V342, N343, F345, F346, L348, and L349. In various embodiments, malonyl-CoA hydrolase enzymes homologous to this consensus sequence (SEQ ID NO:4) contain at least a plurality of these conserved amino acids, often a majority of these conserved amino acids, and sometimes all of these conserved amino acids.

Some amino acids in this consensus sequence (SEQ ID NO:4) contribute to activity and conserved across all members of the class. Malonyl-CoA hydrolase enzymes in the Bacillus EC 3.1.2.4 class contain six active site residues involved with hydrolase activity: (i) three active site amino acid residues (G65, G66, G116) of the consensus sequence believed to contribute to the formation of an oxyanion hole responsible for stabilizing the enolate anion intermediate derived from the malonyl-CoA substrate; (ii) two amino acid residues (E139, D147) of the consensus sequence contribute to acyl-CoA hydrolysis; and (iii) a mutated amino acid (X191) (of SEQ ID NO: 4) believed to contribute to malonyl-CoA substrate binding. Of these six residues, then, five are present in the consensus sequence (SEQ ID NO:4), and the sixth, X191 provides a malonyl CoA hydrolase of this disclosure capable of producing malonate in a recombinant host cell. The six residues from the consensus sequence (G65, G66, G116, E139, D147, X191) correspond to G65, G66, G116, E139, D147, and E91 (typically mutated to X1), respectively, in Bacillus thuringiensis subsp. finitimus (strain YBT-020) FOPNG8 used to illustrate this disclosure in Example 1.

Non-limiting examples of enzymes suitable for malonyl-CoA hydrolysis homologous to the consensus sequence (SEQ ID NO:4) and encoded by cloned or synthesized nucleic acids provided by this disclosure include mutant enzymes containing at least one mutation illustrated by the group of mutant enzymes consisting of Bacillus cereus (strain Q1) B9IZZ9 (E91S), B9IZZ9 (E91A), B9IZZ9 (E91H), B9IZZ9 (E91K), B9IZZ9 (E91R), B9IZZ9 (E91Q); Bacillus thuringiensis subsp. finitimus (strain YBT-020) FOPNG8 (E91S), FOPNG8 (E91A), FOPNG8 (E91H), FOPNG8 (E91K), FOPNG8 (E91R), FOPNG8 (E91Q); Bacillus cereus (strain ATCC 14579/DSM 31) Q81DR3, Q81DR3 (E91S), Q81DR3 (E91A), Q81DR3 (E91H), Q81DR3 (E91K), Q81DR3 (E91R), Q81DR3 (E91Q); Bacillus cereus (strain ZK/E33L) Q63BK8, Q63BK8 (E91S), Q63BK8 (E91A), Q63BK8 (E91H), Q63BK8 (E91K), Q63BK8 (E91R), Q63BK8 (E91Q).

### Pseudomonas EC 3.1.2.4 Malonyl-CoA Hydrolase Consensus Sequence

This disclosure provides a Pseudomonas EC 3.1.2.4 malonyl-CoA hydrolase consensus sequence (SEQ ID NO:5), and in various embodiments, suitable malonyl-CoA hydrolases for use in the methods of this disclosure have at least 75% identity to this Pseudomonas EC 3.1.2.4 malonyl-CoA hydrolase consensus sequence. In various embodiments, the malonyl-CoA hydrolases suitable for use in the methods of this disclosure have 80%, 90%, or 95% or more identity to this Pseudomonas EC 3.1.2.4 consensus sequence. Proteins having homology to this consensus sequence Pseudomonas EC 3.1.2.4 proteins UniProt ID: F5KBQ4 (80% identity), UniProt ID: A6VAN3 (81% identity), UniProt ID: A4XS22 (81% identity), UniProt ID: F6AA82 (75% identity), UniProt ID: E2XN63 (84% identity), UniProt ID: F2KE35 (85% identity), UniProt ID: C3KDS5 (83% identity), UniProt ID: F8G3B7 (86% identity), UniProt ID: G8PYD2 (85% identity), UniProt ID: Q4KGS1 (82% identity), UniProt ID: Q3KGL5 (85% identity), UniProt ID: BOKV51 (86% identity), UniProt ID: BIJ4J2 (86% identity), UniProt ID: A5W8H3 (86% identity), UniProt ID: Q88N06 (86% identity), UniProt ID: Q115T5 (84% identity), UniProt ID: F8H1A4 (77% identity), UniProt ID: A4VIV7 (77% identity), and UniProt ID: Q91515 (81% identity). In some embodiments, a Pseudomonas EC 3.1.2.4 malonyl-CoA hydrolase with equal to or greater than 75% identity to the consensus sequence SEQ ID NO:5 is expressed in a recombinant host cell and used to produce malonate in accordance with this disclosure.

Highly conserved amino acids in this consensus sequence (SEQ ID NO:5) are M1, E6, G13, R15, 116, A19, L21, D22, A23, L27, N28, A29, L30, L32, P33, M34, 135, L38, W45, A46, C53, V54, L56, R57, G58, N59, G60, K62, A63, F64, C65, A66, G67, G68, V70, L73, C77, P81, G82, P85, L87, A88, F91, F92, Y96, R97, L98, H103, P106, K107, P108, C111, W112, H114, G115, V117, G119, G120, G121, M122, G123, L124, Q126, R131, 1132, V133, T134, P135, R138, L139, M141, P142, E143, 1146, G147, L148, D151, V152, G153, S155, F157, L158, R160, P162, G163, L165, G166, L167, F168, L171, N177, D180, A181, D183, L184, L186, A187, D188, R189, Q195, Q196, L199, L203, Q205, N207, W208, E210, Q215, L216, S218, L219, A222, P232, L237, R239, R240, D244, L247, D248, A258, D267, L269, G280, P282, V288, W289, Q291, R294, R296, L298, S299, L300, E307, Y308, S311, L312, N313, C314, R316, H317, P318, F320, E322, G323, V324, R325, A326, R327, L328, D330, D332, P335, W337, W339, P346, A352, H353, and F354. In various embodiments, malonyl-CoA hydrolase enzymes homologous to this consensus sequence (SEQ ID NO:5) contain at least a plurality of these conserved amino acids, often a majority of these conserved amino acids, and sometimes all of these conserved amino acids.

Some amino acids in this consensus sequence (SEQ ID NO:5) contribute to activity and conserved across all members of the class. Malonyl-CoA hydrolase enzymes in the Pseudomonas EC 3.1.2.4 class contain six conserved active site residues that contribute to hydrolase activity (i) three active site amino acid residues (G67, G68, G120) of the consensus sequence believed to contribute to the formation of an oxyanion hole responsible for stabilizing the enolate anion intermediate derived from an acyl-CoA substrate; (ii) two amino acid residues (E143, D151) of the consensus sequence believed to contribute to acyl-CoA hydrolysis; and (iii) amino acid X195 (of SEQ ID NO:5) is believed to contribute to malonyl-CoA substrate binding. In various embodiments of this disclosure, the wild-type glutamic acid residue (E95) is (has been) mutated to a polar or positively charged amino acid (i.e. R, H, K, S, T, N, Q, Y) to produce X195 and provide a malonyl CoA hydrolase of this disclosure capable of producing malonate in a recombinant host cell. The six residues from the consensus sequence (G67, G68, G120, E143, D151, X195) correspond to G67, G68, G120, E143, D151, and E95 (typically mutated to X1), respectively, in Pseudomonas aeruginosa (strain ATCC 15692/PAO1/1C/PRS 101/LMG 12228) F6AA82-2 used to illustrate this disclosure in Example 1.

Non-limiting examples of enzymes suitable for malonyl-CoA hydrolysis homologous to the consensus sequence (SEQ ID NO:5) and encoded by cloned or synthesized nucleic acids provided by this disclosure include mutant enzymes containing at least one mutation illustrated by the group of mutant enzymes consisting of Pseudomonas aeruginosa (strain ATCC 15692/PAO1/1C/PRS 101/LMG 12228) F6AA82-2 (E95S), F6AA82-2 (E95A), F6AA82-2 (E95H), F6AA82-2 (E95K), F6AA82-2 (E95R), F6AA82-2 (E95Q); Pseudomonas fluorescens WH6 E2XN63-1 (E95S), E2XN63-1 (E95A), E2XN63-1 (E95H), E2XN63-1 (E95K), E2XN63-1 (E95R), E2XN63-1 (E95Q); Pseudomonas mendocina (strain ymp) A4XS22-1 (E95S), A4XS22-1 (E95A), A4XS22-1 (E95H), A4XS22-1 (E95K), A4XS22-1 (E95R), A4XS22-1 (E95Q); Pseudomonas putida (strain F1/ATCC 700007) A5W8H3-1 (E95S), A5W8H3-1 (E95A), A5W8H3-1 (E95H), A5W8H3-1 (E95K), A5W8H3-1 (E95R), A5W8H3-1 (E95Q).

In various embodiments of this disclosure the malonyl-CoA hydrolase is E95S mutation of F6AA82-2 from Pseudomonas aeruginosa (strain ATCC 15692/PAO1/1C/PRS 101/LMG 12228), E2XN63-1 from Pseudomonas fluorescens WH6, A4XS22-1 from Pseudomonas mendocina (strain ymp) or A5W8H3-1 from Pseudomonas putida (strain F1/ATCC 700007) as illustrated in Example 1.

### Bacterial EC 3.1.2.4 Malonyl-CoA Hydrolase Consensus Sequence

Despite Bacillus and Pseudomonas being evolutionarily distant (i.e. Bacillus is gram-positive and Pseudomonas is gram-negative), there is sequence conservation between the Bacillus EC 3.1.2.4 and Pseudomonas EC 3.1.2.4 proteins, The present disclosure provides a malonyl-CoA hydrolase consensus sequence for bacterial EC 3.1.2.4 acyl-CoA hydrolases (SEQ ID NO: 6). Proteins homologous to the bacterial EC 3.1.2.4 malonyl-CoA hydrolase consensus sequence typically possess a plurality (or a majority or all) of the highly conserved amino acids from this sequence, which conserved amino acids are selected from the group consisting of L53, L59, N60, L62, M66, L88, F97, C98, A99, G100, G101, F124, F125, Y129, K140, P141, G148, G152, G153, G154, G156, L157, T167, M174, P175, E176, 1179, G180, D184, V185, G186, L191, L210, D219, A226, P333, N364, F375, E377, D385, and P390. A suitable malonyl-CoA hydrolasc provided by this disclosure that is homologous to this consensus sequence will comprise the active site amino acids that contribute to malonyl-CoA hydrolysis (G100, G101, G153, E176, and D184) of the consensus sequence, as well as a X1128, where the wild-type glutamic acid residue (E128) is (has been) mutated to a polar or charged amino acid (i.e. R, H, K, S, T, N, Q, Y) and is capable of producing malonate in a recombinant host cell.

Malonyl-CoA Hydrolases Derived from Malonyl CoA: ACP Transacylases

In yet other embodiments of this disclosure, the malonyl-CoA hydrolase selected from the group malonyl CoA: ACP transacylases (EC 2.3.1.39), containing any or all of the following amino acid modifications: S92, S92C, H201, H201N, R117, R117D, R117E, R117N, R117Y, R117G, R117H, Q11, Q11D, Q1IE, Q1IN, Q11Y, Q11G, Q11H, L93, L93A, L93V, L931, L93F, L93S, L93G. These positions are based on Escherichia coli malonyl CoA: ACP transacylases, FabD.

In some embodiments of this disclosure the malonyl CoA: ACP transacylase is E. coli FabD. Yeast cells expressing a heterologous FabD containing the following combinations of mutations S92C/L91V/R117H, L911/R117Y/A246E, Q80L/L91S/R117G, and L911/R117Y produce malonic acid at levels higher than cells not expressing these mutant proteins.

### Expression Vectors

In various aspects of the present disclosure, the recombinant host cell has been modified by “genetic engineering” to produce a recombinant malonyl-CoA hydrolase enzyme and malonate. The host cell is typically engineered via recombinant DNA technology to express heterologous nucleic acids that encode a malonyl-CoA hydrolase, which is either a mutated version of a naturally occurring acyl-CoA hydrolase or transacylase or a non-naturally occurring malonyl-CoA hydrolase prepared in accordance with one of the consensus sequences provided herein or is a naturally occurring acyl-CoA hydrolase with malonyl-CoA hydrolase activity that is either overexpressed in the cell in which it naturally occurs or is heterologously expressed in a cell in which it does not naturally occur.

Nucleic acid constructs of the present disclosure comprise expression vectors that comprise nucleic acids encoding one or more malonyl-CoA hydrolase enzymes. The nucleic acids encoding the enzymes are operably linked to promoters and optionally other control sequences such that the subject enzymes are expressed in a host cell containing the expression vector when cultured under suitable conditions. The promoters and control sequences employed depend on the host cell selected for the production of malonate. Thus, this disclosure provides not only expression vectors but also nucleic acid constructs useful in the construction of expression vectors. Methods for designing and making nucleic acid constructs and expression vectors generally are well known to those skilled in the art and so are only briefly reviewed herein.

Nucleic acids encoding the malonyl-CoA hydrolase enzymes can be prepared by any suitable method known to those of ordinary skill in the art, including, for example, direct chemical synthesis and cloning. Further, nucleic acid sequences for use in this disclosure can be obtained from commercial vendors that provide de novo synthesis of the nucleic acids.

A nucleic acid encoding the desired enzyme can be incorporated into an expression vector by known methods that include, for example, the use of restriction enzymes to cleave specific sites in an expression vector, for example, plasmid, thereby producing an expression vector of this disclosure. Some restriction enzymes produce single stranded ends that may be annealed to a nucleic acid sequence having, or synthesized to have, a terminus with a sequence complementary to the ends of the cleaved expression vector. The ends are then covalently linked using an appropriate enzyme, for example, DNA ligase. DNA linkers may be used to facilitate linking of nucleic acids sequences into an expression vector.

A set of individual nucleic acid sequences can also be combined by utilizing polymerase chain reaction (PCR)-based methods known to those of skill in the art. For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other. Extension of this overlap by DNA polymerase produces a molecule in which the original sequences are “spliced” together. In this way, a series of individual nucleic acid sequences may be joined and subsequently transduced into a host cell simultaneously. Thus, expression of each of the plurality of nucleic acid sequences is affected.

A typical expression vector contains the desired nucleic acid sequence preceded and optionally followed by one or more control sequences or regulatory regions, including a promoter and, when the gene product is a protein, ribosome binding site, for example, a nucleotide sequence that is generally 3-9 nucleotides in length and generally located 3-11 nucleotides upstream of the initiation codon that precede the coding sequence, which is followed by a transcription terminator in the case of E. coli or other prokaryotic hosts. See Shine et al., Nature 254:34 (1975) and Steitz, in Biological Regulation and Development: Gene Expression (ed. R. F. Goldberger), vol. 1, p. 349 (1979) Plenum Publishing, N.Y. In the case of eukaryotic hosts like yeast a typical expression vector contains the desired nucleic acid coding sequence preceded by one or more regulatory regions, along with a Kozak sequence to initiate translation and followed by a terminator. See Kozak, Nature 308:241-246 (1984).

Regulatory regions or control sequences include, for example, those regions that contain a promoter and an operator. A promoter is operably linked to the desired nucleic acid coding sequence, thereby initiating transcription of the nucleic acid sequence via an RNA polymerase. An operator is a sequence of nucleic acids adjacent to the promoter, which contains a protein-binding domain where a transcription factor can bind. Transcription factors activate or repress transcription initiation from a promoter. In this way, control of transcription is accomplished, based upon the particular regulatory regions used and the presence or absence of the corresponding transcription factor. Non-limiting examples for prokaryotic expression include lactose promoters (LacI repressor protein changes conformation when contacted with lactose, thereby preventing the LacI repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Non-limiting examples of promoters to use for eukaryotic expression include pTDH3, pTEF1, pTEF2, pRNR2, pRPL18B, PREV1, pGAL1, pGAL10, pGAPDH, pCUP1, pMET3, pPGK1, pPYK1, pHXT7, pPDC1, pFBA1, pTDH2, pPGI1, pPDC1, pTPI1, pENO2, pADH1, and pADH2. As will be appreciated by those of ordinary skill in the art, these and other expression vectors or elements may be used in the present disclosure, and this disclosure is not limited in this respect.

Although any suitable expression vector may be used to incorporate the desired sequences, readily available expression vectors include, without limitation: plasmids, such as pESC, pTEF, p414CYC1, p414GALS, pSC101, pBR322, pBBRIMCS-3, pUR, pEX, pMR100, pCR4, pBAD24, pUC19, pRS series; and bacteriophages, such as M13 phage and λ phage. Of course, such expression vectors may only be suitable for particular host cells or for expression of particular malonyl-CoA hydrolases. One of ordinary skill in the art, however, can readily determine through routine experimentation whether any particular expression vector is suited for any given host cell or protein. For example, the expression vector can be introduced into the host cell, which is then monitored for viability and expression of the sequences contained in the vector. In addition, reference may be made to the relevant texts and literature, which describe expression vectors and their suitability to any particular host cell. In addition to the use of expression vectors, strains are built where expression cassettes are directly integrated into the host genome.

The expression vectors are introduced or transferred, for example by transduction, transfection, or transformation, into the host cell. Such methods for introducing expression vectors into host cells are well known to those of ordinary skill in the art. For example, one method for transforming E. coli with an expression vector involves a calcium chloride treatment wherein the expression vector is introduced via a calcium precipitate.

For identifying whether a nucleic acid has been successfully introduced or into a host cell, a variety of methods are available. For example, a culture of potentially transformed host cells may be separated, using a suitable dilution, into individual cells and thereafter individually grown and tested for expression of a desired gene product of a gene contained in the introduced nucleic acid. For example, an often-used practice involves the selection of cells based upon antibiotic resistance that has been conferred by antibiotic resistance-conferring genes in the expression vector, such as the amp, gpt, neo, and hyg genes.

Typically, a host cell of this disclosure will have been transformed with at least one expression vector. When only a single expression vector is used, the vector will typically contain a malonyl-CoA hydrolase gene. Once the host cell has been transformed with the expression vector, the host cell is cultured in a suitable medium containing a carbon source, such as a sugar (for example, glucose). As the host cell is cultured, expression of the enzyme(s) for producing malonate occurs.

If a host cell of this disclosure is to comprise more than one heterologous gene, the multiple genes can be expressed from one or more vectors. For example, a single expression vector can comprise one, two, or more genes encoding one, two, or more malonyl-CoA hydrolase enzyme(s) and/or other proteins providing some useful function, for example improved malonate yield, titer, and/or productivity. The heterologous genes can be contained in a vector replicated episomally or in a vector integrated into the host cell genome, and where more than one vector is employed, then all vectors may replicate episomally (extrachromosomally), or all vectors may integrate, or some may integrate and some may replicate episomally. Chromosomal integration is typically used for cells that will undergo sustained propagation, for example, cells used for production of malonate for industrial applications. While a “gene” is generally composed of a single promoter and a single coding sequence, in certain host cells, two or more coding sequences may be controlled by one promoter in an operon. In some embodiments, a two or three operon system is used.

In some embodiments, the coding sequences employed have been modified, relative to some reference sequence, to reflect the codon preference of a selected host cell. Codon usage tables for numerous organisms are readily available and can be used to guide sequence design. The use of prevalent codons of a given host organism generally improves translation of the target sequence in the host cell. As one non-limiting example, in some embodiments the subject nucleic acid sequences will be modified for yeast codon preference (see, for example, Bennetzen et al., J. Biol. Chem. 257:3026-3031 (1982)). In some embodiments, the nucleotide sequences will be modified for E. coli codon preference (see, for example, Nakamura et al., Nucleic Acids Res. 28:292 (2000)).

Nucleic acids can be prepared by a variety of routine recombinant techniques. Briefly, the subject nucleic acids can be prepared from genomic DNA fragments, cDNAs, and RNAs, all of which can be extracted directly from a cell or recombinantly produced by various amplification processes including but not limited to PCR and rt-PCR. Subject nucleic acids can also be prepared by a direct chemical synthesis.

The nucleic acid transcription levels in a host microorganism can be increased (or decreased) using numerous techniques. For example, the copy number of the nucleic acid can be increased through use of higher copy number expression vectors comprising the nucleic acid sequence, or through integration of multiple copies of the desired nucleic acid into the host microorganism's genome. Non-limiting examples of integrating a desired nucleic acid sequence onto the host chromosome include recA-mediated recombination, lambda phage recombinase-mediated recombination and transposon insertion. Nucleic acid transcript levels can be increased by changing the order of the coding regions on a polycistronic mRNA or breaking up a polycistronic operon into multiple poly- or mono-cistronic operons each with its own promoter. RNA levels can be increased (or decreased) by increasing (or decreasing) the strength of the promoter to which the protein-coding region is operably linked. Illustrative techniques for plasmid design and assembly to afford malonate production are provided in Example 1.

The translation level of a desired polypeptide sequence in a host microorganism can also be increased in a number of ways. Non-limiting examples include increasing the mRNA stability, modifying the ribosome binding site (or Kozak) sequence, modifying the distance or sequence between the ribosome binding site (or Kozak sequence) and the start codon of the nucleic acid sequence coding for the desired polypeptide, modifying the intercistronic region located 5′ to the start codon of the nucleic acid sequence coding for the desired polypeptide, stabilizing the 3′-end of the mRNA transcript, modifying the codon usage of the polypeptide, altering expression of low-use/rare codon tRNAs used in the biosynthesis of the polypeptide. Determination of preferred codons and low-use/rare codon tRNAs can be based on a sequence analysis of genes derived from the host microorganism.

The polypeptide half-life, or stability, can be increased through mutation of the nucleic acid sequence coding for the desired polypeptide, resulting in modification of the desired polypeptide sequence relative to the control polypeptide sequence. When the modified polypeptide is an enzyme, the activity of the enzyme in a host may be altered due to increased solubility in the host cell, improved function at the desired pH, removal of a domain inhibiting enzyme activity, improved kinetic parameters (lower Km or higher Kcat values) for the desired substrate, removal of allosteric regulation by an intracellular metabolite, and the like.

Altered/modified enzymes can also be isolated through random mutagenesis of an enzyme, such that the altered/modified enzyme can be expressed from an episomal vector or from a recombinant gene integrated into the genome of a host microorganism.

### Additional Modifications and Fermentation Conditions for Improved Malonate Production

In other aspects of this disclosure, increased malonate yield, titer, and/or productivity is achieved by employing host cells provided by this disclosure that have been genetically modified in ways other than, or in addition to, introduction of a heterologous malonyl-CoA hydrolase and/or by employing fermentation conditions provided by certain methods of this disclosure. In brief, embodiments of the recombinant host cells of this disclosure can comprise genetic modifications that increase acetyl-CoA biosynthesis, increase malonyl-CoA biosynthesis, decrease malonate catabolism increase secretion of malonate from the host cell, increase host cell tolerance to malonate, increase catabolism of various carbon sources and/or any combination of the foregoing.

Genetic Modifications and Fermentation Conditions that Increase Acetyl-CoA Biosynthesis

In accordance with embodiments of this disclosure, increased malonate titer, yield, and/or productivity can be achieved by genetic modifications that increase acetyl-CoA biosynthesis, and this disclosure provides enzymes that increase acetyl-CoA biosynthesis, vectors for expressing enzymes that increase acetyl-CoA biosynthesis, host cells expressing enzymes that increase acetyl-CoA biosynthesis and increase malonate titer, yield, and/or productivity, and methods relating thereto. As described above, malonate is produced by hydrolysis of malonyl-CoA, which, can be produced from acetyl-CoA; thus, increases in acetyl-CoA biosynthesis can improve malonate production.

One route by which acetyl-CoA is produced is by an acetyl-CoA synthetase (EC 6.2.1.1), which catalyzes the formation of acetyl-CoA from acetate and coenzyme A (CoA). Embodiments of this disclosure provide recombinant host cells suitable for producing malonate in accordance with the methods of this disclosure comprising one or more heterologous acetyl-CoA synthetase (ACS) enzymes that increase malonate titer, yield, and/or productivity relative to a host cell not comprising a heterologous acetyl-CoA synthetase. Non-limiting examples of suitable ACS enzymes arc S. cerevisiae ACS1 (GenBank: AAC04979.1) and ACS2 (GenBank: CAA97725.1). In some embodiments, a recombinant host cell comprising S. cerevisiae acetyl-CoA synthetase ACS1 and/or ACS2 is used to increase malonate titer, yield, and/or productivity. In other embodiments, a recombinant host cell comprising an acetyl-CoA synthetase selected from the group consisting of Salmonella enterica Acs, Escherichia coli AcsA, and Bacillus subtilis AcsA is used to increase malonate yield, titer, and/or productivity. Other acetyl-CoA synthetases can be expressed in a recombinant host cell producing malonate in accordance with this disclosure to increase malonate yield, titer, and/or productivity.

A second route through which acetyl-CoA is produced is by a pyruvate dehydrogenase complex, which catalyzes the formation of acetyl-CoA from pyruvate. Embodiments of this disclosure provide recombinant host cells suitable for producing malonate in accordance with the methods of this disclosure that comprise one or more heterologous pyruvate dehydrogenase complex enzymes that increase malonate titer, yield, and/or productivity relative to a host cell not comprising a heterologous pyruvate dehydrogenase complex enzyme. Non-limiting examples of suitable pyruvate dehydrogenase complex enzymes include S. cerevisiae PDA1, PDB1, LAT1, LPD1, and PDX1. In some embodiments of this disclosure, malonate yield, titer, and/or productivity are increased in a recombinant host cell used to produce malonate by expressing one or more pyruvate dehydrogenase enzymes selected from the group consisting of S. cerevisiae PDA1, PDB1, LAT1, LPD1, and PDX1. Other pyruvate dehydrogenase enzymes can be expressed in a recombinant host cell producing malonate in accordance with this disclosure to increase malonate yield, titer, and/or productivity.

A third route through which acetyl-CoA is produced is by a heterologous ethanol catabolic pathway comprising enzymes catalyzing the conversion of ethanol to acetyl-CoA. Compared to malonate, ethanol is a less expensive chemical, and host cells producing malonate and expressing an ethanol catabolic pathway can convert ethanol to malonate. An alcohol dehydrogenase (EC 1.1.1.1) catalyzes conversion of ethanol to acetaldehyde. Non-limiting examples of suitable alcohol dehydrogenase enzymes include those selected from the group consisting of S. cerevisiae ADH2, E. coli AdhP, H. sapiens ADH1A, H. sapiens ADH1B, and H. sapiens ADH1C. In addition to the alcohol dehydrogenase, an ethanol catabolic pathway also comprises either an acetaldehyde dehydrogenase (acylating; EC 1.2.1.10), or an aldehyde dehydrogenase (EC 1.2.1.3) and an acetyl-CoA synthetase (EC 6.2.1.1). An acetaldehyde dehydrogenase (acylating) catalyzes the conversion of acetaldehyde to acetyl-CoA, an aldehyde dehydrogenase catalyzes the conversion of acetaldehyde to acetate, and an acetyl-CoA synthase, as described above, catalyzes the formation of acetyl-CoA from acetate and CoA. Non-limiting examples of suitable acetaldehyde dehydrogenases (acylating) include those selected from the group consisting of E. coli MhpF, E. coli AdhE, Pseudomonas sp CF600 DmpF, and Pseudomonas putida TodL. Non-limiting examples of aldehyde dehydrogenases include S. cerevisiae ALD2, ALD3, ALD4, ALD5, and ALD6; and H. sapiens ALD1, ALD2, ALD4, and ALD10. Non-limiting examples of acetyl-CoA synthetase enzymes include S. cerevisiae ACS1, S. cerevisiae ACS2, and E. coli Acs.

Embodiments of the present disclosure provide recombinant host cells suitable for producing malonate in accordance with the methods of this disclosure comprising one or more heterologous ethanol catabolic pathway enzymes that increase malonate yield, titer, and/or productivity relative to host cells not comprising the heterologous ethanol catabolic pathway enzyme(s). In some embodiments, the heterologous ethanol catabolic pathway enzymes are an ethanol dehydrogenase and an acetaldehyde dehydrogenase (acylating). In some embodiments, the heterologous ethanol catabolic pathway enzymes are S. cerevisiae ADH2 ethanol dehydrogenase and E. coli MhpF acetaldehyde dehydrogenase (acylating). In some embodiments, a heterologous S. cerevisiae ADH2 and E. coli MhpF are expressed in recombinant E. coli expressing a heterologous S. cerevisiae EHD3 malonyl-CoA hydrolase. In other embodiments, a heterologous S. cerevisiae ADH2 and E. coli MhpF are expressed in recombinant S. cerevisiae expressing a heterologous S. cerevisiae EHD3 malonyl-CoA hydrolase. In other embodiments, a heterologous S. cerevisiae ADH2 and E. coli MhpF are expressed in a recombinant oleaginous yeast expressing a heterologous S. cerevisiae EHD3 malonyl-CoA hydrolase. In other embodiments, the heterologous ethanol catabolic pathway enzymes are S. cerevisiae ADH2 ethanol dehydrogenase and Pseudomonas sp. CF600 DmpF acetaldehyde dehydrogenase (acylating). In some embodiments, a heterologous S. cerevisiae ADH2 and Pseudomonas sp. CF600 DmpF are expressed in recombinant E. coli expressing a heterologous S. cerevisiae EHD3 malonyl-CoA hydrolase. In some embodiments, a heterologous S. cerevisiae ADH2 and Pseudomonas sp. CF600 DmpF are expressed in recombinant S. cerevisiae expressing a S. cerevisiae EHD3 malonyl-CoA hydrolase. In some embodiments, a heterologous S. cerevisiae ADH2 and Pseudomonas sp. CF600 DmpF are expressed in a recombinant oleaginous yeast expressing a heterologous S. cerevisiae EHD3 malonyl-CoA hydrolase. In other embodiments, the heterologous ethanol catabolic pathway enzymes are S. cerevisiae ADH2 ethanol dehydrogenase and Pseudomonas putida TodL acetaldehyde dehydrogenase (acylating). In some embodiments, a heterologous S. cerevisiae ADH2 and Pseudomonas putida TodL are expressed in recombinant E. coli expressing a heterologous S. cerevisiae EHD3 malonyl-CoA hydrolase. In some embodiments, a heterologous S. cerevisiae ADH2 and Pseudomonas putida TodL are expressed in recombinant S. cerevisiae expressing a heterologous S. cerevisiae EHD3 malonyl-CoA hydrolase. In some embodiments, a heterologous S. cerevisiae ADH2 and Pseudomonas putida TodL are expressed in a recombinant oleaginous yeast expressing a heterologous S. cerevisiae EHD3 malonyl-CoA hydrolase. In other embodiments, the heterologous ethanol catabolic pathway enzymes are one or more alcohol dehydrogenase selected from the group containing S. cerevisiae ADH2, E. coli AdhP, H. sapiens ADH1A, H. sapiens ADH1B, and/or H. sapiens ADH1C and one or more acetaldehyde dehydrogenase (acylating) selected from the group containing E. coli MhpF, E. coli AdhE, Pseudomonas sp CF600 DmpF, and Pseudomonas putida TodL. Other alcohol dehydrogenase enzymes and acetaldehyde dehydrogenase (acylating) enzymes can be expressed in a recombinant host cell suitable for producing malonate in accordance with the methods of this disclosure to increase malonate yield, titer, and/or productivity.

In other embodiments, the heterologous ethanol catabolic pathway enzymes are an ethanol dehydrogenase, an aldehyde dehydrogenase, and an acetyl-CoA synthetase. In some embodiments, the heterologous ethanol catabolic pathway enzymes are a S. cerevisiae ALD2 alcohol dehydrogenase, a S. cerevisiae ALD2 aldehyde dehydrogenase, and a S. cerevisiae ACS1 acetyl-CoA synthetase. In other embodiments, the heterologous ethanol catabolic pathway enzymes are a S. cerevisiae ALD2 alcohol dehydrogenase, a S. cerevisiae ALD2 aldehyde dehydrogenase, and a S. cerevisiae ACS2 acetyl-CoA synthetase. In other embodiments, the heterologous ethanol catabolic pathway enzymes are a S. cerevisiae ALD2 alcohol dehydrogenase, a S. cerevisiae ALD6 aldehyde dehydrogenase, and a S. cerevisiae ACS1 acetyl-CoA synthetase. In other embodiments, the heterologous ethanol catabolic pathway enzymes are a S. cerevisiae ALD2 alcohol dehydrogenase, a S. cerevisiae ALD6 aldehyde dehydrogenase, and a S. cerevisiae ACS2 acetyl-CoA synthetase. In other embodiments, the heterologous ethanol catabolic pathway enzymes are one or more alcohol dehydrogenases selected from the group containing S. cerevisiae ADH2, E. coli AdhP, H. sapiens ADH1A, H. sapiens ADH1B, and/or H. sapiens ADH1C, one or more aldehyde dehydrogenases selected from the group containing S. cerevisiae ALD2, S. cerevisiae ALD3, S. cerevisiae ALD4, S. cerevisiae ALD5, S. cerevisiae ALD6, H. sapiens H. sapiens ALD1, H. sapiens ALD2, H. sapiens ALD4, and/or H. sapiens ALD10, and one or more acetyl-CoA synthetases selected from the group containing S. cerevisiae ACS1, S. cerevisiae ACS2, and/or E. coli Acs.

In some embodiments, recombinant host cells suitable for producing malonate according to the methods of this disclosure comprise a heterologous ethanol catabolic pathway enzyme and convert endogenously produced ethanol into acetyl-CoA and increase malonate yield, titer, and/or productivity. In other embodiments, ethanol is exogenously added to the fermentation broth and recombinant host cells suitable for producing malonate according to the methods of this disclosure comprise a heterologous ethanol catabolic pathway enzyme and convert exogenously added ethanol into acetyl-CoA and increase malonate yield, titer, and/or productivity. When exogenously added to the fermentation broth, ethanol is added to obtain a minimal concentration of 1% ethanol volume/volume and is typically added to the fermentation broth to obtain a concentration between 1-15% volume/volume.

Increased cytosolic pools of acetyl-CoA is a fourth route to increase malonate biosynthesis; in numerous plant and animal cells, but not S. cerevisiae, ATP citrate lyase (EC 2.3.3.8) is the primary enzyme responsible for cytosolic acetyl-CoA biosynthesis. In more detail, acetyl-CoA in the mitochondrion is condensed with oxaloacetate to form citrate through the activity of citrate synthase. Subsequently, citrate is transported from the mitochondrion into the cytosol where ATP citrate lyase catalyzes the formation of acetyl-CoA, oxaloacetate, and ADP. While S. cerevisiae does not contain a native ATP citrate lyase, suitable heterologous ATP citrate lyase enzymes have been described in oleaginous yeast strains (see, for example, Boulton et al., J. Gen. Microbiol. 127:169-176 (1981)). Embodiments of the present disclosure provide recombinant host cells comprising one or more heterologous nucleic oleaginous yeast ATP citrate lyase enzymes. Non-limiting examples of oleaginous yeast ATP citrate lyase enzymes include those selected from the group of oleaginous yeasts consisting of Candida curvata, Cryptococcus albidus, Lipomyces lipofer, Rhodospiridium toruloides, Rhodotorula glutanis, Trichosporon cutaneum, Yarrowia lipolytica, and the like. In various embodiments, the recombinant host cell comprises a heterologous nucleic acid encoding an ATP citrate lyase. In various embodiments, the ATP citrate lyase is from an organism selected from the group consisting of Candida curvata, Cryptococcus albidus, Lipomyces lipofer, Rhodospiridium toruloides, Rhodotorula glutanis, Trichosporon cutaneum, Yarrowia lipolytica.

Acetyl-CoA biosynthesis can also be increased in accordance with this disclosure by altering expression of one or more nucleic acids encoding proteins affecting fatty acid storage or catabolism the present disclosure provides host cells comprising genetic modifications of one or more nucleic acids encoding proteins affecting fatty acid storage and catabolismin Saccharomyces cerevisiae, these proteins include SNF2, IRA2, PRE9, PHO90, SPT21, POX1, ANT1, FOX3, PAS1, PAS3, ARE1, ARE2, DGA1, LRO1, ACL1, MAE1, GLC3, GLG1, GLG2, PAT1, and PEX11.

In some embodiments of this disclosure, the host cell comprises genetic modifications affecting expression and/or activity of proteins involved in fatty acid catabolism For example, most host cells will naturally degrade fatty acids, hydroxy fatty acids and many diacids through beta-oxidation pathways. Beta-oxidation occurs, in most cases, by activating free fatty acid groups to CoA thioesters with acyl-CoA ligases. The acyl-CoA intermediate is further oxidized and degraded-proceeding through a 2,3 enoyl-CoA, 3-hydroxyacyl-CoA, and 3-ketoacyl-CoA- and subsequent cleavage results in production of acetyl-CoA and an acyl-CoA shortened by two carbons relative to the initial substrate. The enzymatic activities required for beta-oxidation are known. The present disclosure provides host cells that possess increased catabolic pathway activity for medium (C4-C8)- and long (>C8)-chain fatty acids, hydroxyl fatty acids, and diacids compared to control host cells. For example, in yeast (for example, Saccharomyces cerevisiae), beta-oxidation occurs in the peroxisome; non-limiting nucleic acid products affecting peroxisomal beta-oxidation are Saccharomyces cerevisiae PAT1 and PEX11. In some embodiments of this disclosure, a host cell modified for increased expression of PAT1 and/or PEX11 is provided for use in the methods herein for the production of malonate.

Genetic Modifications and Fermentation Conditions that Increase Malonyl-CoA Biosynthesis

In accordance with this disclosure, increased malonate titer, yield, and/or productivity can be achieved through increased malonyl-CoA biosynthesis, and this disclosure provides host cells, vectors, enzymes, and methods relating thereto. Malonyl-CoA is produced in host cells through the activity of an acetyl-CoA carboxylase (EC 6.4.1.2) catalyzing the formation of malonyl-CoA from acetyl-CoA and carbon dioxide. This disclosure provides recombinant host cells for producing malonate that express a heterologous acetyl-CoA carboxylase (ACC). In some embodiments, the host cell is a S. cerevisiae cell comprising a heterologous S. cerevisiae acetyl-CoA carboxylase ACC1 or an enzyme homologous thereto. In some embodiments, the host cell modified for heterologous expression of an ACC such as S. cerevisiae ACC1 is further modified to eliminate ACC1 post-translational regulation by genetic modification of S. cerevisiae SNF1 protein kinase or an enzyme homologous thereto. This disclosure also provides a recombinant host cell suitable for producing malonate in accordance with this disclosure that is an E. coli cell that comprises a heterologous nucleic acid coding for expression of E. coli acetyl-CoA carboxylase complex proteins AccA, AccB, AccC and AccD or one or more enzymes homologous thereto. In accordance with this disclosure, additional acetyl-CoA carboxylases can be heterologously expressed to increase malonate biosynthesis.

In various embodiments of this disclosure, expression of BirA, biotin-[acetylCOA carboxylase]holoenzyme synthetase, is coexpressed with E. coli acetyl-CoA carboxylase complex proteins AccA, AccB, AccC and AccD to enhance the activity of the ACC complex and result in an increase in malonate production. In various embodiments of this disclosure, S. cerevisiae ACC1 is further modified to eliminate ACC1 post-translational regulation by introducing serine to alanine mutations at any, all, or any combination of the following residues; S10, S233, S430, S1114, S1145, S1148, S1157, S1159, S1162, S1163, S1169. In some embodiments of this disclosure, the acetyl-CoA carboxylase used is from Yarrowia lipolytica CLIB122. In additional embodiments of this disclosure, this enzyme is coexpressed with a biotin-[acetyl-CoA carboxylase]holoenzyme synthetase, also derived from this organism. In additional embodiments of this disclosure, the acetyl-CoA carboxylases and biotin-[acetylCoA carboxylase]holoenzyme synthetase encoding genes are dtsR1 accBc and derived from Corynebacterium glutamicum. In additional embodiments of this disclosure, these genes are derived from a yeast strain including, but not limited to those of the genera, Candida, Pichia, or any of the other yeast herein. In various embodiments of this disclosure, the host cell producing malonate expresses any combination of these acetyl-CoA carboxylases and biotin-[acetylCoA carboxylase]holoenzyme synthetase enzymes.

In some embodiments of this disclosure, a host cell suitable for producing malonate according to the methods of this disclosure comprises genetic modifications affecting expression and/or activity of proteins involved in fatty acid biosynthesis. Malonyl-CoA is naturally a substrate in the biosynthesis of fatty acids, and diversion of malonyl-CoA to fatty acid production decreases the ability for the host cell to produce malonate. This disclosure provides recombinant host cells for producing malonate that express a heterologous fatty acid synthase (FAS) multienzyme complex. Temperature sensitive mutations of S. cerevisiae fatty acid synthase complex are known (see, Knobling et al., Eur. J. Biochem., 59:415-421 (1975)). Expression of a heterologous, temperature sensitive fatty acid synthase complex allows diversion of malonyl-CoA to fatty acid biosynthesis to be controlled by the temperature at which the host cell is cultured. In some embodiments, the host cell is a S. cerevisiae cell comprising S. cerevisiae fatty acid synthases FAS1 and FAS2 or enzymes homologous thereto. In some embodiments of this disclosure, FAS 1 and FAS2 enzymes are temperature-sensitive FAS1 or FAS2 enzymes.

In addition to genetic modification of the host cell, fatty acid biosynthesis can be decreased through addition of a FAS inhibitor to the cell culture media. For example, the FAS inhibitor cerulenin forms a covalent bond with the active site cysteine C1305 in the S. cerevisiae ketoacyl synthase domain of the FAS complex, inhibiting enzyme activity (Johansson et al., PNAS, 105:12803-12808 (2008)). Cerulenin is not only effective in inhibiting S. cerevisiae FAS activity, but is generally an inhibitor of FAS complexes containing a Cys-His-His or Cys-His-Asn catalytic triad in the ketoacyl synthase domain. In some embodiments, cerulenin is added to the fermentation broth to a final concentration between 5 mg/L and 100 mg/L to inhibit fatty acid biosynthesis and increase malonate production in recombinant host cells producing malonate in accordance with the methods of this disclosure. In various embodiments of a method of this disclosure, a FAS inhibitor is added to fermentation broth containing recombinant host cells producing malonate. In some embodiments of a method of this disclosure, the FAS inhibitor is cerulenin. In some embodiments of the method of this disclosure, cerulenin is supplemented in the fermentation broth at a concentration between 5 mg/L and 100 mg/L. In other embodiments of a method of this disclosure, the fatty acid synthase complex inhibitor is selected from a group consisting of platensimycin, thiolactomycin, and triclosan.

One of the substrates for acetyl-CoA carboxylase is carbon dioxide and increasing the carbon dioxide partial pressure in the fermentation broth promotes formation of malonyl-CoA. In certain embodiments, the fermentation broth has a minimum dissolved carbon dioxide pressure of 0.01 atmospheres, and an increase in dissolved carbon dioxide partial pressure above this threshold is desirable. The fermentation broth should typically contain between 0.1 and 1 atmospheres dissolved carbon dioxide partial pressure. The dissolved carbon dioxide partial pressure in the fermentation broth may be increased to above saturating conditions, or above 1 atmosphere dissolved carbon dioxide. In some embodiments of a method of this disclosure, the dissolved carbon dioxide partial pressure in the fermentation broth is increased to between 0.1 and 1 atmospheres. In some embodiments of the method of this disclosure, carbon dioxide partial pressure is increased through addition of carbonates or bicarbonates to fermentation broth. For example, and without limitation, calcium carbonate can be added to the fermentation broth to increase dissolved carbon dioxide partial pressure. In other embodiments of the method of this disclosure, the fermentation is run in a pressurized vessel that contains carbon dioxide at above atmospheric pressure. In other embodiments of the method of this disclosure, carbon dioxide gas is sparged into the fermentation broth. The gas mixture being sparged may contain other gases if the added components do not interfere with host cell growth or malonate production. It may be advantageous to co-localize the source of the carbon dioxide gas with the malonate fermentation. For example, and without limitation, gaseous carbon dioxide resulting from various fermentation processes (for example, ethanol, isobutanol, 3-hydroxypropionate, etc.), chemical processes (for example, downstream malonate synthetic chemistry), or energy generation (for example, coal or natural gas powerplants) may be pumped into fermentation broth from malonate producing host cells to increase the carbon dioxide partial pressure.

Genetic Modifications that Decrease Malonate Catabolism

In accordance with this disclosure, increased malonate titer, yield, and/or productivity can be achieved by decreasing malonate catabolism and this disclosure provides host cells, vectors, enzymes, and methods relating thereto. One metabolic pathway by which malonate is catabolized in a host cell is through the activity of an acyl-CoA synthetase catalyzing the conversion malonate and Coenzyme A to malonyl-CoA. In some embodiments of this disclosure, a recombinant host cell suitable for producing malonate in accordance with the methods of this disclosure comprises a genetic modification resulting in the deletion, attenuation, or modification of one or more nucleic acids encoding for an acyl-CoA synthetase. In some embodiments of this disclosure, the recombinant host cell is yeast and the one or more acyl-CoA synthetases are selected from the group consisting of FAA1, FAA2, FAA3, FAA4, LSC1, and LSC2. In other embodiments of this disclosure, the recombinant host cell is E. coli and the one or more acyl-CoA synthetases are selected from the group consisting of FadD, FadK, FadI, SucC, SucD, and YahF.

Genetic Modifications that Increase Malonate Secretion From the Host Cell

In accordance with this disclosure, increased malonate titer, yield, and/or productivity can be achieved by increasing malonate transport into the fermentation broth, and this disclosure provides host cells, materials, and methods relating thereto. In some embodiments of this disclosure, the recombinant host cell suitable for use in the methods of this disclosure is a S. cerevisiae cell that comprises a heterologous nucleic acid coding for expression of an S. cerevisiae transport protein selected from the group consisting of PDR5, PDR10, PDR11, PDR12, PDR15 and PDR18. In some embodiments of this disclosure, the recombinant host cell suitable for producing malonate in accordance with the methods of this disclosure is an E. coli cell that comprises a heterologous nucleic acid coding for expression of E. coli DcuC.

Genetic Modifications that Increase Host Cell Tolerance to Malonate

In accordance with this disclosure, increased malonate titer, yield, and/or productivity can be achieved by increasing host cell tolerance to malonate, and this disclosure provides host cells, materials, and methods relating thereto. High concentrations of malonate can competitively inhibit succinate dehydrogenase (EC 1.3.5.1) activity (see Slater, Methods Enzymol. 10:48-57 (1967)). The present disclosure is based, in part, on the discovery that mutant succinate dehydrogenase enzymes exhibit a lower competitive inhibition by malonate. For example, S. cerevisiae succinate dehydrogenase SDH1 residues E300, R331, and R442 are involved in substrate (for example, succinate) recognition. Increasing the size of the SDH1 active site decreases competitive inhibition by malonate while still allowing the enzyme to maintain activity toward the native substrate, succinate. In specific, introduction of one or more mutations selected from the group consisting of E300D, R331K or R331H, and R442K and R442H decreases competitive inhibition of SDH1 by malonate. In some embodiments, a recombinant host cell expressing an SDH1 with point mutation R300D is used to produce malonate in accordance with this disclosure. In other embodiments, a recombinant host cell expressing an SDH1 with point mutation R331K or R331H is used to produce malonate in accordance with this disclosure. In other embodiments, a recombinant host cell expressing an SDH1 with point mutation R442K or R442H is used to produce malonate in accordance with this disclosure.

Genetic Modifications that Increase Catabolism of Various Carbon Sources

In the methods of this disclosure, carbon feedstocks are utilized for production of malonate. Suitable carbon sources include, without limitation, those selected from the group consisting of purified sugars (for example, dextrose, sucrose, xylose, arabinose, lactose, etc.); plant-derived, mixed sugars (for example, sugarcane, sweet sorghum, molasses, cornstarch, potato starch, beet sugar, wheat, etc.), plant oils, fatty acids, glycerol, cellulosic biomass, alginate, ethanol, carbon dioxide, methanol, and synthetic gas (“syn gas”). The Examples provided herein demonstrate the production of malonic acid in accordance with the methods of this disclosure using a variety of carbon sources.

This disclosure provides host cells comprising genetic modifications that increase malonate titer, yield, and/or productivity through the increased ability to catabolize non-native carbon sources. Wild type S. cerevisiae cells are unable to catabolize pentose sugars, lignocellulosic biomass, or alginate feedstocks. In some embodiments, this disclosure provides a S. cerevisiae cell comprising a heterologous nucleic acid encoding enzymes enabling catabolism of pentose sugars useful in production of malonate as described herein. In other embodiments, the heterologous nucleic acid encodes enzymes enabling catabolism of lignocellulosic feedstocks. In yet other embodiments of this disclosure, the heterologous nucleic acid encodes enzymes increasing catabolism of alginate feedstocks.

### Production and Purification of Malonate and Its Esters Via a Soluble Ammonium Salt

The methods described herein relate to the production and purification of bio-based diester derivatives of malonic acid from malonates, such as ammonium malonates. These malonates may be derived from a fermentation broth of a microorganism that is able to produce malonate from a fermentable carbon source. In some embodiments, this method may occur at a commercially viable level. Ammonium malonate can be produced by fermentation following methods described in PCT App. Pub. WO 2015200545 (incorporated herein by reference).

The general methods described herein include producing malonic acid and/or diester derivatives of malonic acid, the methods comprising: (a) culturing a recombinant host cell under conditions suitable for production of malonate in an aqueous solution (fermentation), (b) recovering the malonate from the fermentation broth, and (c) producing DMM and AMS, using a series of steps. This disclosure also provides purified DMM and AMS compositions produced in accordance with the methods of this disclosure.

### Fermentation

Embodiments of the methods provided herein may comprise the step of fermenting a microorganism capable of producing malonate in the presence of a fermentable carbon source under suitable fermentation conditions to obtain an aqueous fermentation broth that comprises aqueous malonate. The microorganism can be any microorganism capable of producing malonate.

In some embodiments, the microbial culture may comprise microorganisms capable of producing malonate from fermentable carbon source(s). Non-limiting examples of microorganisms include those selected from the group comprising Pichia kudriavzevii, Saccharomyces cerevisiae, Escherichia coli, and derivatives thereof. A preferred microorganism is a Pichia kudriavzevii strain.

As described herein, fermentation may comprise the inoculation of a microorganism capable of producing ammonium malonate in the presence of a fermentable carbon source under suitable fermentation conditions into fermentation broth containing a fermentable carbon source. The fermentation conditions may be altered as needed for the organism used. Either a synthetic or a natural medium can be used so long as the microorganism is capable of growth in the medium. In one example, Pichia kudriavzevii is grown in an appropriate medium. Suitable media for fermenting typically depend on the choice of microorganism used. A typical nutrient medium for Pichia kudriavzevii may contain a fermentable carbon source, a nitrogen source, a phosphorous source, inorganic salts, and optionally other trace organic nutrients, including vitamins that can improve the health and growth of the microorganism.

In some embodiments of preparing bio-based compositions from fermentation, a growth vessel, typically a fermenter, can be used to grow a microbial culture that is subsequently used for the production of malonate, malonic acid, and/or diester derivatives of malonic acid-containing fermentation broth. Such fermentation vessels are known in the art.

Fermentation methodology is well-known in the art and can be carried out in a batch-wise, continuous or semi-continuous manner. In some embodiments, the fermentation occurs at a commercially viable level. In some embodiments, the fermentation and subsequent purification of bio-based diester derivatives of malonic acid can take place in a vessel capable of holding the desired volume. In some embodiments, this vessel is a reactor. The vessel can be capable of holding from 1 gallon to 100,000 gallons or more. Such vessels are known in the art.

In some embodiments, the microbial culture (i.e., fermentation broth) may comprise a fermentable carbon source for example, and, optionally, a source of nitrogen, phosphorous, and additional media components such as vitamins, salts, and other materials that can improve cellular growth and/or product formation, and water. These components may be fed into a fermenter to regulate or promote growth and sustenance of the microbial culture. In some embodiments, the microbial culture may be grown under aerobic conditions provided by sparging an oxygen containing gas (for example, air or the like). In some embodiments, the hydroxide or carbonate bases of calcium, ammonium, or sodium can be provided for pH control during the growth of the microbial culture.

The fermentable carbon source may be any fermentable carbon source. Non-limiting examples of fermentable carbon sources include glucose, glucose monohydrate, sucrose, maltose, glycerol, ethanol, acetic acid, and mixtures thereof. In one embodiment, the fermentable carbon source is glucose. In another embodiment, the fermentable carbon source is sucrose.

The nitrogen source may be any assimilable nitrogen source. Either synthetic or natural nitrogen sources, or a mixture of synthetic and natural nitrogen sources, may be used. Non-limiting examples of synthetic assimilable nitrogen sources include ammonia, ammonium salts (for example, ammonium hydroxide, ammonium sulfate, ammonium carbonate, and ammonium phosphates), urea, and nitrates. Non-limiting examples of natural nitrogen sources include yeast extract and peptone.

Suitable fermentation conditions are typically dependent on the choice of microorganism used (see, for example, Krahe, M. 2003. Biochemical Engineering. Ullmann's Encyclopedia of Industrial Chemistry). Fermentation conditions comprise a suitable growth media, suitable fermentation method, suitable temperature, suitable oxygenation, and suitable pH. Examples of fermentation conditions and media recipes are disclosed in U.S. patent application Ser. No. 14/386,272.

Suitable temperatures for fermenting typically depend on the choice of microorganism used. In embodiments in which the microorganism is a yeast, a suitable temperature for fermenting can be from 15° C. to 45° C., to 40° C., to 35° C., or to 30° C.; more preferably from 20° C. to 35° C., or to 30° C.; and most preferably about 30° C.

To produce malonate, oxygen is transferred into the fermentation broth; in other words, there is a positive oxygen transfer rate (OTR). Microbial production of malonic acid results in the concomitant formation of the redox cofactors NADH and/or NADPH, which are recycled to NAD⁺ and NADP⁺, respectively, to maintain the redox balance for cell health and efficient malonic acid production. Molecular oxygen is typically the electron acceptor used to recycle NAD(P) H back to NAD(P)⁺ and suitable oxygenation of the fermentation broth can efficiently produce malonic acid in a fermentation. Oxygenation of the fermentation broth may be generally achieved by pumping in either atmospheric air (i.e., air that is about 21% molecular oxygen) or oxygen-enriched air. The rate at which oxygen is transferred into the fermentation broth (oxygen transfer rate, or OTR), expressed as mmol-O₂/L/hr, describes the oxygenation of the fermentation broth. In many embodiments of the present disclosure, the fermentation OTR is at least 5 mmol/L/h, at least 10 mmol/L/hr, at least 20 mmol/L/hr, at least 30 mmol/L/hr, at least 40 mmol/L/hr, or at least 50 mmol/L/hr. In many embodiments of the present disclosure, the fermentation OTR is 150-200 mmol/L/h. In some embodiments, enriched air (air blended with pure O₂ to an O₂ content of ˜ 35% typically) is employed to obtain higher OTRs.

Fermentations at neutral or near neutral pH values (i.e., from about pH 6 to about pH 8) have an increased risk of contamination by undesired, non-malonic acid producing microbes from the external environment. Therefore, it may be preferable for at least a portion, and often a majority, and sometimes all, of a fermentation to be operated at a pH value less than or equal to pH 7. However, at the same time, a high concentration of malonic acid at a low pH is toxic to most microorganisms and results in decreased growth rate, cell viability, production. Thus, a suitable fermentation pH depends on both the choice of the microorganism used (i.e., its ability to grow and produce malonic acid at a lower fermentation pH) and the concentration of fully protonated malonic acid in solution. Generally speaking, to decrease malonic acid-induced toxicity it is often desirable to culture the malonic acid producing microorganism at a pH at least as high as the pKa of the first carboxylic acid of malonic acid, and often times at a pH of at least as high as the pKa of the second carboxylic acid of malonic acid. Doing so minimizes the concentration of fully protonated malonic acid the cells are exposed to and thus minimizes malonic acid-induced toxicity.

In some embodiments, the preferred pH of the fermentation is kept around pH 5.0. In one embodiment, calcium hydroxide is used to control the pH. The fermentation process may result in a mixture of cells, soluble malonate, and a variety of soluble organic compounds (for example, calcium acetate and calcium succinate). When carrying out the fermentation at pH 5.0, the concentration of succinate at 11 g/L or below can be monitored. If the concentration of succinate is too high, it will form an insoluble salt, which will be difficult to separate from the malonate.

In some embodiments, various methods can be used to decrease the concentration of succinate in the fermentation broth, including adjustment of the fermentation oxygen transfer rate and/or modification of the fermentation process such that the majority of succinate produced during the fermentation is re-consumed at the end of the run.

A second method useful for decreasing the concentration of succinate concentration in the fermentation broth is to adjust the fermentation process such that any succinate produced is re-consumed by the engineered microbe. Since succinate is a small-molecule required in nearly all microbe's native metabolism, most microbes, including P. kudriavzevii as well as other yeast cells, will re-consume succinate once more preferred carbon sources (for example, glucose) have been depleted from the fermentation broth. The operator can allow the concentration glucose to decrease to about zero g/L and the engineered microbe will begin re-consuming the succinate in the broth. While this method can be employed at any point during a fermentation it is typically used at the end of the fermentation. Additionally, this method is particularly advantageous when producing malonic acid since most microbes (including P. kudriavzevii and other yeast) cannot re-consume malonic acid; thus, the amount of malonic acid produced in the fermentation is not decreased when using this approach.

The fermentation pH can be controlled by the addition of various inorganic bases at the beginning and/or throughout the course of the fermentation, and the choice of the fermentation base affects the pKa values for the two carboxylic acid groups. In the presence of a monovalent cation (for example, a sodium cation when sodium hydroxide is used as a base) the two carboxylic acid pKa values are about 2.83 and 5.69. The apparent pKa of the carboxylic acids shifts when using calcium hydroxide as base.

In the presence of certain alkaline earth metals, the second carboxylic acid pKa value decreases. For example, in the presence of calcium, the second carboxylic acid pKa of malonic acid decreases to about 3.15. It is therefore possible to ferment a malonic acid producing microorganism at a lower fermentation pH without observing malonic acid-induced toxicity when neutralizing the broth with a calcium base or other alkaline earth metal bases as compared to bases for which the cation is monovalent. In many embodiments, the fermentation pH is less than or equal to pH 7 for all or part of the fermentation. In some embodiments, the fermentation pH is less than or equal to pH 6 for all or part of the fermentation. In some embodiments, the fermentation pH is less than or equal to pH 5 for all or part of the fermentation.

In some embodiments, the microbial culture (i.e., fermentation broth) may comprise glucose as a fermentable carbon source, NH₄OH as a source of nitrogen, and additional batch media components such as vitamins, salts, and other materials (such as antifoam feed) that can improve cellular growth and/or product formation. These components may be fed into a production fermenter to regulate or promote growth and sustenance of the microbial culture. In some embodiments, the fermenter volume is 500 m³. In some embodiments, the microbial culture may be grown under aerobic conditions provided by sparging an oxygen containing gas (for example, sterile air or the like).

Embodiments of the fermentation method as disclosed herein are not particularly limited and suitable fermentation methods include batch, fed-batch, and continuous fermentations. However, in order to obtain a larger yield of ammonium malonate, a fed batch culture where the fermentable carbon source is sequentially added over time may be typically used. In many embodiments of the present disclosure, the fermentation method is a fed-batch fermentation method.

### Ammonium Malonate Concentration in Fermentation Broth

In some embodiments, the present disclosure provides methods to isolate malonate produced biologically. Isolating malonate in accordance with these methods involves separating the malonate salts produced from at least part or all of the fermentation broth, host cells, and parts thereof, from which malonate is produced. Malonate may be purified, i.e., to more than 50% purity on a w/w basis, in accordance with this disclosure from the fermentation broth and/or from the producing cell in which any naturally occurring or recombinant host cell (for example E. coli, S. cerevisiae, oleaginous yeast, and the like) producing malonate is grown, i.e., the host cell is not limited to a recombinant host cell of this disclosure.

Biosynthesized malonate can be produced intracellularly and/or secreted into the culture medium. Intracellularly produced malonate is typically secreted into the culture medium using a membrane transporter, as described above. If not secreted, malonate can be removed from the host cell by chemical, enzymatic, or mechanical cell lysis. Malonate can be recovered from the cells, from the fermentation broth, or both. Without being bound by theory, it may be advantageous to not lyse the cells intentionally before separating them, since the portion of malonate contained in the biomass is very small relative to the extracellular portion, and because other impurities including trehalose and arabitol are released into the supernatant if the cells are lysed. If the cell is engineered to secrete malonate, one can opt to recover the malonate only from the fermentation broth or one can opt to recover it both from the product broth and from the cell (i.e., by lysing the cell). If the cell is not engineered to secrete malonate one can lyse the host cell to isolate the malonate therein.

In some embodiments, the purification methods of this disclosure comprise the step of recovering the malonate produced, wherein the recovering step is concurrent or subsequent to the culturing step. In some embodiments, the malonate is purified from the fermentation broth and the host cells. In other embodiments, the host cells are separated from the fermentation broth, lysed, and then malonate is recovered from the host cells. In other embodiments, the host cells are lysed in the fermentation broth and malonate is recovered from the lysed cells and fermentation broth. In other embodiments, the cells are separated from the supernatant and washed to recover extracellular malonate, while minimizing lysis so that impurities contained in intracellular fluid are minimized in the resulting recovered malonate.

In some embodiments of the purification methods of this disclosure, the fermentation medium is concentrated to increase the working concentration of malonate and decrease the volume of liquid for processing. In various embodiments of the purification methods of this disclosure, this concentration is achieved by evaporation, including evaporation under vacuum.

In some embodiments, the microorganism is Pichia kudriavzevii. In some embodiments, the fermentation of Pichia kudriavzevii is carried out in the presence of ammonium hydroxide. In some embodiments, the ammonium hydroxide is concentrated to 3M. In some embodiments, the ammonium hydroxide is 10 M NH₄OH. In some embodiments, the ammonium hydroxide is about 13 M NH₄OH. Gaseous NH₃ is employed in some embodiments.

In some embodiments, when the fermentation has been carried out for the desired amount of time, the fermentation broth may be collected and cleared of soluble and insoluble impurities. Non-limiting examples of soluble impurities in include salts, metabolic byproducts produced by the cell, and unconsumed carbohydrates. The primary insoluble impurity present in fermentation broth is cells (i.e., biomass). Cells are particularly problematic in that their occurrence in downstream purification steps can decrease malonic acid yields and product quality through cell lysis and release of various intracellular compounds (for example metabolites, proteins, and cell debris). Therefore, it is preferable to separate the soluble malonate from both the fermentation broth and cells present in the fermentation broth.

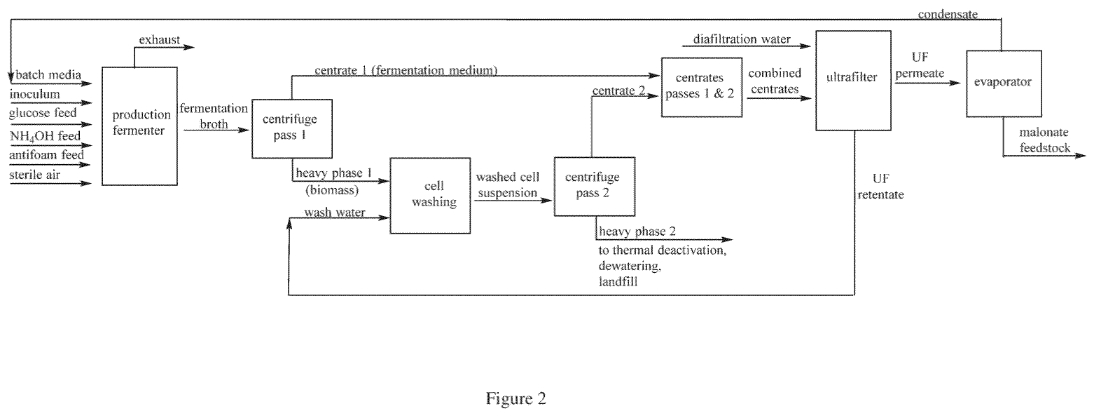

In some embodiments, insoluble cells and cell debris may be processed through a series of steps as depicted in FIG. 2. For example, the fermentation broth may first be centrifuged to generate a first centrate (fermentation medium or “centrate 1”) and a heavy phase (biomass or “heavy phase 1”). In some embodiments, the heavy phase may be washed with a cell wash (for example, water) to create a washed cell suspension and liberate any residual malonate therefrom. In some embodiments, this washed cell suspension may then be centrifuged to generate a second heavy phase (“heavy phase 2”) and a second centrate (“centrate 2”). In some embodiments, heavy phase 2 may later be used for thermal deactivation, dewatering, and/or discarded. In some embodiments, centrate 1 and centrate 2 may be combined with diafiltration water and subjected to ultrafiltration. In some embodiments, ultrafiltration will result in an ultrafiltration (“UF”) permeate and a UF retentate. In some embodiments, the UF retentate may be recycled with wash water to the earlier washing step of heavy phase 1. In some embodiments, the UF retentate will be next passed to an evaporator to produce a concentrated ammonium malonate solution. In some embodiments, centrate 1 and centrate 2 may be combined with diafiltration water and instead subjected to nanofiltration. In some embodiments, the concentrated malonate is concentrated ammonium malonate. In some embodiments, the cells may be continuously washed as an integral part of the separation process, for example by performing the cell separation with a drum filter, belt filter, or basket centrifuge with integral washing capabilities.

In some embodiments, insoluble cells and cells debris can be separated by centrifugation. If desired, the process may be carried out by proceeding directly to filtration. Centrifugation can be carried out in a decanter centrifuge, preferably the horizontal type, or hydrocyclones. Hydrocyclones may be used to separate the insoluble biomass from soluble ammonium malonate. Hydrocyclones are quite efficient at removing cellular debris from the fermentation broth. Hydrocyclones separate materials of different sizes and/or densities using a centrifugal force. The centrifugal force is generated by introduction of the slurry into the cyclone under pressure; larger and/or denser particles are pushed to the outside of the cone while smaller and/or less dense particles are kept closer to the center. The vortex finder draws the majority of the water and fine particles to the overflow while the larger/denser materials are drawn out of the apex. Hydrocyclones work best if the range of total suspended solids is kept at 5-25%. Higher % TSS may result in the loss of CaM to the overflow. If desired the fermentation broth may be diluted or concentrated to bring the % TSS within the 5-25% range. Reproducibility will be enhanced if the process is generally carried out using the same % TSS each time. In some embodiments,

In one embodiment, a single hydrocyclone may be used. In one embodiment, a series of three hydrocyclones may be used to separate soluble ammonium malonate from the majority of the cells. Efficient cell removal is a parameter to be monitored.

Depending on the scale of the operation, different routes may be followed for the first steps of concentrating ammonium malonate in the fermentation broth. In smaller scale or benchtop operations, the underflows from the hydrocyclones can be taken after centrifugation and sent to a tank for re-suspension. Wash water from the process can be used to bring the total suspended solids (% TSS) to a level of 20-25%. The use of wash water may help dilute out the cells and the impurities in the fermentation broth. In larger operations such as continuous or semi-continuous manufacturing, the wash water may be obtained after horizontal vacuum belt (HVBF) unit operation. In either case, reusing wash water may be beneficial.

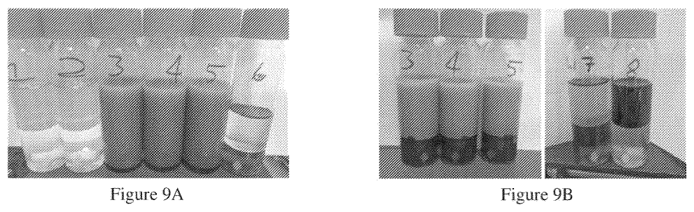

In some embodiments, synthetic samples of malonates may separate quickly and cleanly, with no emulsion (See FIG. 9A, Vial 1). In contrast, malonates in the fermentation broth produced according to the methods described herein do not generally separate quickly and cleanly, even after one or more rounds of centrifugation. In some embodiments, the addition of anti-foaming agents (for example, Struktol B2121) before or after reactive extraction in conjunction with sodium sulfate fails to resolve the emulsion (See FIG. 9A, Vial 4 (added before the reaction) and Vial 5 (added after the reaction)). In some embodiments, allowing the pre-reactive extraction reaction mixture to sit overnight may partially resolve the emulsion. (See FIG. 9B, Vials 3-5). In some embodiments, ultrafiltration prior to reactive extraction allows the phases of the reaction mixture to separate cleanly (See FIG. 9B, Vials 7 & 8).

### Ultrafiltration (UF)

In some embodiments, the methods provided herein comprise the step of removing impurities from the soluble malonate. Impurities may react with malonic acid and reduce final yields, or may contribute to the bio-based malonic acid being of lower purity and having more limited industrial utility.

In some embodiments, malonate in the fermentation broth may have impurities derived from the fermentation culture and/or recombinant host cells. In other embodiments, malonate may be free or essentially free of impurities from the recombinant host cells. The malonate can be isolated or purified to a degree such that any impurities present do not interfere in the subsequent use of the malonate. For example, if the subsequent use is as an industrial chemical, such as a chemical to be used in a polymerization reaction, then the malonate is essentially free of impurities when any remaining impurities would not interfere with the use of the malonate in a polymerization reaction. Typically, malonate used for polymerization reactions has a purity of at least 95% w/w or higher. If the malonate is to be used as a fuel, such as a fuel to be used in a combustion reaction, then the compound is essentially free of impurities when any impurities remaining would not interfere with the use of the malonate as a fuel. If the malonate is used as an animal feed, then the malonate is essentially free of impurities when any impurities remaining would not interfere with the use of the material as animal feed. When malonate is used as an animal feed, one may opt to recover the biomass containing malonate from the fermentation broth and use the biomass as animal feed.

In some embodiments, ultrafiltration and/or nanofiltration may be used to separate out certain salts, sugars, color forming bodies, and other organic compounds present in the fermentation medium (aka ammonium malonate concentrate). In ultrafiltration, the fermentation medium may be filtered through a membrane having pore sizes ranging from 0.005 microns to 0.5 microns. In nanofiltration, the malonic acid solution may be filtered through a membrane having pore sizes ranging from 0.0005 microns to 0.005 microns, equating to a molecular weight cut-off of about 100 Daltons to about 2,000 Daltons. Nanofiltration can be useful for removing divalent and multivalent ions, maltose and other disaccharides (for example, sucrose), polysaccharides, and other complex molecules with a molecular weight greater than malonic acid. In some embodiments, cation and/or anion exchange chromatography may be used to remove specific salts and charged compounds present in the malonate solution.

Non-limiting examples of other impurities present in the malonate solution include color bodies, hydrophobic compounds, excess cations, volatile compounds (for example, odorants), chloride ions, and uncatabolized carbohydrates. Many of these impurities can be removed by filtration, chromatography, steam stripping, and/or a combination of these unit operations. In some embodiments, activated carbon may be used to remove trace impurities including color and hydrophobic compounds. In some embodiments, cation exchange resin may be used to remove calcium left over from the gypsum precipitation as well as any other residual cationic species remaining in solution. Using chromatography columns to elute the input solution through both activated carbon and cation exchange resin has been found to reduce final impurity concentrations further than in batch applications with the same amounts of material. In some embodiments, a flow rate of 3 BV/hour achieves an optimal resistance time for absorption. In some embodiments, the use of 1% granular activated carbon on a malonic acid weight basis has also been shown to sufficiently remove color impurities.

In some embodiments, the methods provided herein may decrease the levels of one or more impurities in the resulting diester derivatives of malonic acid. By removing impurities or making a de novo preparation lacking the impurities, through the compositions containing diester derivatives of malonic acid disclosed herein, more control is held over cure speed and in the improvement of the hardness of resins and polymers in downstream applications. Non-limiting examples of impurities removed to a degree from malonic acid using the methods disclosed herein, or avoided by making a de novo preparation lacking the impurities, comprise diethyl sulfate (DES), cyanoacetic acid esters, cyanoacetate, other cyano-compounds, chloro compounds including chloroacetate and ethyl chloroacetate, 2-propanone, sodium cyanide, and acetic acid, (acetyloxy)-ethyl ester.

The use of the disclosed improved methods will avoid the synthesis of diester derivatives of malonic acid containing higher levels of impurities, and may increase final yields of diester derivatives of malonic acid and/or downstream products. In some embodiments, the use of the disclosed methods may result in the purification or synthesis of bio-based diester derivatives of malonic acid of higher purity and with broader industrial utility.

### Preparation of Diester Derivatives of Malonic Acid and AMS

The methods provided herein may further comprise the step of preparing diester derivatives of malonic acid from bio-based malonic acid. Diester derivatives of malonic acid may include, among other lower alkyl (i.e., lowere alkanol derived) esters, diethyl malonate (“DEM”) or dimethyl malonate (“DMM”).

The present disclosure provides a method for the production of DMM and such other dialkyl esters, using a series of continuous process operations. In some embodiments, the malonate feedstock for the method is a crude concentrate of an aqueous salt of malonic acid, such as, ammonium malonate. In some embodiments, the method utilizes sulfuric acid to acidify the malonate and to catalyze its esterification with methanol to produce DMM. In some embodiments, a reactive extraction using toluene as a hydrophobic extractant is part of the method, and provides the means for separating the DMM from bulk water, which can enable high conversion and separation of the product from water soluble impurities. In some embodiments, the use of a high salt concentration crude concentrate of malonate feedstock can increase the partitioning of the product into toluene. In some embodiments, the method produces ammonium sulfate or AMS. In some embodiments, the method includes unit operations to enable a high yield of DMM to be obtained, to recover and recycle toluene and excess methanol, and to process the raffinate from the reactive extraction to produce an AMS byproduct with minimal residual solvents and other organic impurities.

In some embodiments of the methods disclosed herein, the malonate feedstock is a result of the previously described fermentation and concentration method steps. In some embodiments, the malonate feedstock is a solution containing soluble malonate salts. In some embodiments, the malonate substrate in the malonate feedstock enters the process as a mixture of diammonium malonate, monoammonium malonate, and malonic acid in an aqueous solution containing organic impurities from the fermentation process used to generate the material. In some embodiments, the pH of the malonate feedstock is about 5 and the feed contains about 1.5 mol ammonium per mol malonate.

### Aqueous Esterification

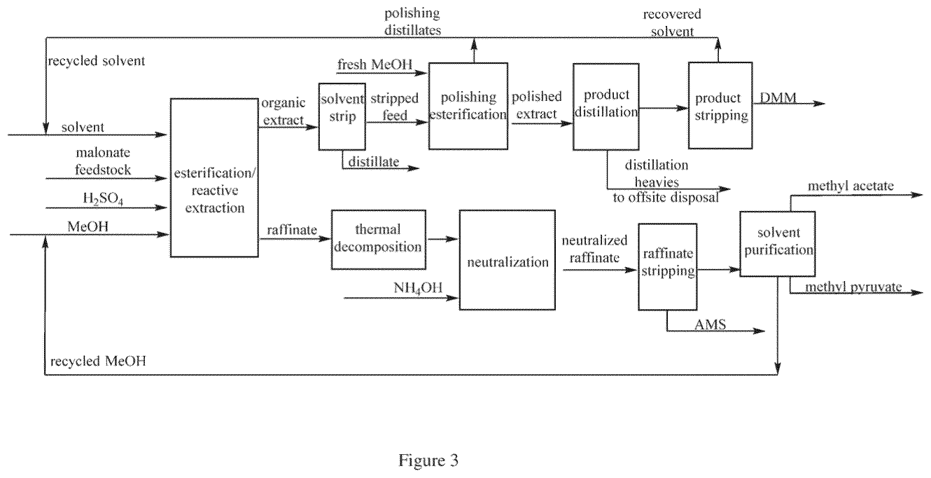

In some embodiments, the methods disclosed herein comprise an aqueous esterification. The acidification of aqueous malonate in a malonate feedstock can produce a significant amount of heat and esterification of malonate can also be mildly exothermic. In some embodiments, aqueous esterification occurs when the malonate feedstock, a lower alkanol such as methanol, and acid such as sulfuric acid are mixed in a continuous stirred tank reactor. In some embodiments, the methanol can be recycled from previous reactions (see FIG. 3). In some embodiments, the methanol is fresh or is not recycled. The unit operation can remove the majority of heat generated by acidification and esterification, and preheats the material before it is fed to the reactive extraction process. This allows the reactive extraction operation to be designed adiabatically with only a modest temperature change. In some embodiments, about two-thirds of the esterification reaction occurs during the aqueous esterification. In some embodiments, the reaction product contains a small amount of malonic acid, and significant amounts of both a monoalkyl malonate such as monomethyl malonate (“MMM”) and a dialkyl malonate such as DMM. Examples of parameters for aqueous esterification are listed in Table 1, below.

TABLE 1
Aqueous Esterification

| | |
|---|---|
| Crude malonate feedstock | 2.75M malonic acid equivalent |
| composition | (concentration specified so that |
| | (NH4)₂SO₄ is 40 wt % after |
| | MeOH removal) |
| | 1.5 mol NH₄⁺ per mol malonate |
| | 50 wt % water |
| | 0.02 mol succinate/mol malonate |
| | 0.005 mol pyruvate/mol malonate |
| | 0.01 mol acetate/mol malonate |
| | Additional components with unspecified |
| | masses include glycerol, arabitol, and |
| | small amounts of other materials |
| Equivalents of methanol fed | 4 mol MeOH/mol malonate |
| (as recycled methanol) | |
| Equivalents of 98% sulfuric | 2.2 mol H₂SO₄/mol malonate |
| acid fed | (1.5 eq for acidification of malonate, |
| | 0.7 eq excess for titrating other species |
| | and catalyzing reaction) |
| Temperature | 70° C. |
| Pressure | 0 psig |
| | (bleed through N2 to maintain inert |
| | headspace/prevent air backflow into |
| | condenser) |
| Residence Time | 30 min |
| Malonate Distribution | 4.6 mol % malonic acid |
| in Product | 34.8 mol % MMM |
| | 60.6 mol % DMM |
| Heat Generation Rate | Approx. 1160 kW |
| | Approx. 93 kJ/mol NH₄ in feedstock |
| | (acidification) |
| | Approx. 16 kJ/mol MeOH consumed |
| | (esterification) |
| Density of Reaction Mixture | 1.2 kg/L @ RT |
| Viscosity of Reaction Mixture | 5 cP @ 70° C. |
| Materials of Construction | Glass-lined reactor |

Without being bound by theory, in the esterification reaction, the malonate is neutralized by acidification with sulfuric acid, generating ammonium bisulfate in the process, as shown below.

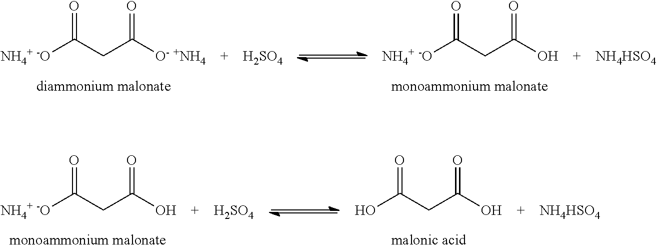

In some embodiments, the malonate feedstock is esterified by contacting with a lower alkanol such as methanol and an acid such as sulfuric acid. This produces MMM and DMM. DMM partitions strongly into toluene, and MMM partitions slightly into toluene. By performing the esterification as a reactive extraction with toluene, the reaction equilibrium is shifted significantly towards DMM. In some embodiments, this can be done in a counter-current reactive extraction to result in a high process yield with an economical amount of solvent consumption. In some embodiments a significant amount of the malonate extracted is in the form of a monoalkyl malonate such as monomethyl malonate. The esterification reactions are shown schematically below.

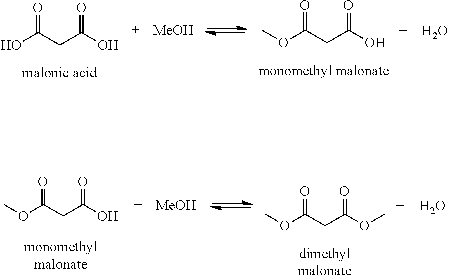

Malonates can undergo irreversible decarboxylation reactions. These reactions may proceed at significant rates under conditions relevant to the product distillation. These reactions can be performed intentionally in a separate part of the process, to eliminate malonates from the inorganic salt byproduct such as AMS. Decarboxylation of MMM produces methyl acetate, and decarboxylation of malonic acid produces acetic acid, as shown below.

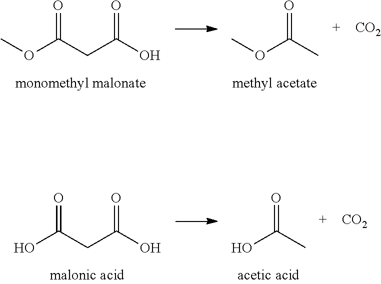

In some embodiments, hydrolysis reactions (the reverse of the esterification reactions shown above) can convert DMM or another dialkyl malonate to MMM or another monoalkyl malonate and the monoalkyl malonate or MMM to malonic acid. In some embodiments, an aqueous mixture of DMM or another dialkyl malonate, MMM or another monoalkyl malonate, and malonic acid can be irreversibly decomposed to a mixture of methyl acetate or another alkyl acetate, acetic acid, and carbon dioxide in the presence of water.

In some embodiments, the malonate feedstock contains some ionized organic acids that undergo similar esterification reactions as malonates. These ionized organic acids include but are not limited to succinate, pyruvate, and acetate. In some embodiments, these ionized organic acids (also called “impurities”) can be present to some extent in both their acid and their methyl ester forms after reactive extraction. For example, dimethyl succinate is a high-boiling impurity which needs to be removed as part of the final product distillation. As another example, methyl pyruvate is a low-boiling impurity which needs to be removed as part of the final product distillation. In some embodiments methyl acetate and acetic acid are produced by neutralization and esterification of acetate in the malonate feedstock, and they are also produced by decarboxylation of malonates during processing as previously described. In some embodiments, methyl acetate can be separated from methanol and purged from the process so that it doesn't accumulate within the process over time, as methanol is repeatedly recycled. In some embodiments, some amounts of organic acids will be allowable in the aqueous AMS byproduct (where they will be present primarily in their ionized ammonium forms). Table 2, below, contains components of consideration in certain embodiments of the disclosed methods.

TABLE 2
Components

| | | Mol. Wt. | Normal BP |
|---|---|---|---|
| Name | Structure | (g/mol) + formula | (° C.) |
| Methyl acetate | 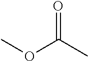 | 74.08 C₃H₆O₂ | 57 |
| Methanol | 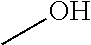 | 32.04 CH₄O | 65 |
| Toluene | 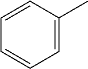 | 92.14 C₇H₈ | 110 |
| Methyl pyruvate | 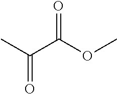 | 102.09 C₄H₆O₃ | 134-137 |
| DMM | 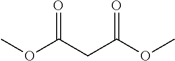 | 132.12 C₅H₈O₄ | 180 |
| Dimethyl succinate | 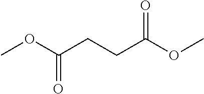 | 146.14 C₆H₁₀O₄ | 200 |

In some embodiments, impurities may be present in allowable quantities in either the product or byproduct stream. Table 3, below, contains certain potential illustrative impurities.

TABLE 3
Potential Impurities

| | | Mol. Wt. | |
|---|---|---|---|
| | | (g/mol) + | Normal |
| Name | Structure | formula | BP (° C.) |
| Methyl-3-methyl-2-oxopentanoate | 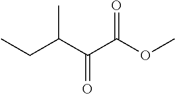 | 144.17 C₇H₁₂O₃ | 142 |
| Methyl-3-hydroxypropanoate | 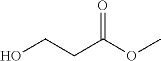 | 104.11 C₄H₈O₃ | 143 |
| Methyl-2-oxobutanoate | 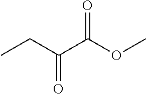 | 116.12 C₅H₈O₃ | 170 |
| Dimethyl methylmalonate | 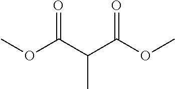 | 146.14 C₆H₁₀O₄ | 176- 177 |
| Dimethyl sulfate | 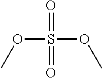 | 126.13 C₂H₆O₄S | 188 |
| Ethyl methyl malonate | 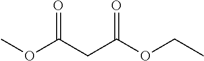 | 146.14 C₆H₁₀O₄ | 190 |
| Dimethyl fumarate | 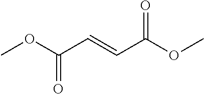 | 144.13 C₆H₈O₄ | 193 |
| Methyl levulinate | 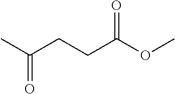 | 130.14 C₆H₁₀O₃ | 194 |
| Dimethyl itaconate | 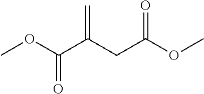 | 158.15 C₇H₁₀O₄ | 208 |
| Process antifoam (Struktol SB-2121) | Polyethyleneglycol | | — |
| Arabitol | 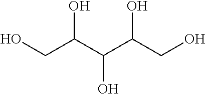 | 158.15 C₅H₁₂O₅ | — |
| Glycerol | 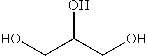 | 92.09 C₃H₈O₃ | — |

In some embodiments, the initial malonate concentration in the malonate feedstock is about 1.3 M, temperature is about 70° C., about 4 mol methanol is added per mol malonate, and about 2.2 mol sulfuric acid is added per mol malonate.

The sensitivity of the reaction rate to temperature can be evaluated by performing experiments at various temperatures (50, 70, and 90° C.) feeding 6 mol of a lower alkanol or MeOH/mol malonate and 2.1 mol of an acid or H₂SO₄/mol malonate.

In some embodiments, the product distribution is stabilized to constant values after ˜20 minutes, and the final concentrations of the various malonates, water, and methanol are used to calculate the equilibrium constants. In some embodiments, the data are fit with a kinetic model assuming the following second-order kinetics:

$${r}_{1} = {k}_{1} ( {c}_{MA} {c}_{MeOH} - ( 1 / {K}_{1} ) {c}_{MMM} {c}_{H 2 O} )$$

$${r}_{2} = {k}_{2} ( {c}_{MMM} {c}_{MeOH} - ( 1 / {K}_{2} ) {c}_{DMM} {c}_{H 2 O} )$$

Here r₁ and r₂ are the net rates of the first and second esterification reactions of malonate, with units of M/h. k₁ and k₂ are the rate constants with units of M⁻¹ h⁻¹. K₁ and K₂ are the dimensionless equilibrium constants for the two reactions. cⱼ are the molar concentrations of the various species.

Example rate constants obtained by fitting kinetic data fit at reactions at 70° C., 9 mol MeOH/mol malonate, 2.2 mol H₂SO₄/mol malonate are listed in Table 4.

TABLE 4
Example Constants

| Parameter | Value |
|---|---|
| Rate constant k₁ | 1.75 M⁻¹ h⁻¹ |
| Rate constant k₂ | 0.60 M⁻¹ h⁻¹ |
| Equilibrium constant K₁ | 8.7 |
| Equilibrium constant K₂ | 2.0 |

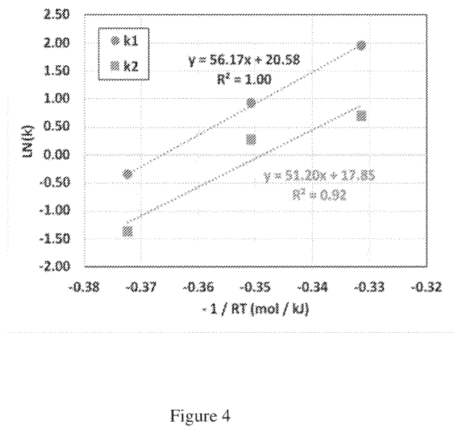

Without being bound by theory, an Arrhenius plot of the rate constants fit at these temperatures can be generated, such as the illustration Arrhenius plot shown in FIG. 4. This plot shows activation energies of 56 and 51 KJ/mol for the first and second esterification reactions, respectively. This plot depicts measurements at 50° C., 70° C., and 90° C. feeding 6 mol MeOH/mol malonate and 2.1 mol H₂SO₄/mol malonate. In some embodiments, the reaction rate constants will roughly double for every increase in temperature of 13-14° C. In some embodiments, the equilibrium constants decrease slightly with increase in temperature.

### Reactive Extraction

In some embodiments, the aqueous esterification methods disclosed herein comprise reactive extraction. In some embodiments, the reactive extraction process results in a high degree of esterification while simultaneously extracting di and mono alkyl (preferably lower alkyl, more preferably C₁-C₃ alkyl) malonates into toluene. In some embodiments, the esterification reaction occurs in the aqueous phase, catalyzed by an acid such as H₂SO₄. An alkanol such as methanol partitions primarily into the aqueous phase, and DMM partitions primarily into the organic solvent such as toluene, so the reaction equilibrium is shifted further towards completion as the extraction progresses. In some embodiments, there is an organic solvent or an immiscible liquid phase and xylene, toluene, anisole, or methyl alkanoate esters compose greater than 10 wt %, or greater than 30 wt %, or greater than 50 wt %, or greater than 70 wt %, or greater than 90 wt % of the immiscible liquid extracting phase. In some embodiments, extraction yield can be increased by (a) improving contacting in the column with larger impellers near bottom/smaller impellers near top, to improve contacting at the bottom while avoiding flooding at the top; and/or (b) increasing temperature. In some embodiments, the system is pressurized.

In some embodiments, reactive extraction comprises a countercurrent reaction column. In some embodiments, reactive extraction steps are carried out using a Scheibel column (e.g., and without limitation with 3-inch inner diameter, 10 L of internal volume, and 60 physical stages). Such an extractor system may utilize Hastelloy impellers, and each impeller can have the same design and size. In some embodiments, the extractor is operated at an agitation rate of 150 rev/min, 200 rev/min, or 250 rev/min. In some embodiments, the organic holdup is only ˜15 vol % during the tests, as measured by stopping feeds and agitation at the end of each run and measuring the amounts of each phase held up in the column. In some embodiments, about 8-12% of the total moles of malonate fed to the column is present in the raffinate when the conditions are: 4 mol lower alkanol such as methanol per mol malonate, 2.2 mol H₂SO₄ per mol malonate, feeds at 60° C., 0.75-1.0 L o-xylene or another organic solvent per L aqueous feed.

In some embodiments, o-xylene is utilized as the organic solvent or the extraction solvent. In some embodiments, toluene is utilized as the organic solvent or the extraction solvent. In some embodiments, anisole is utilized as the organic solvent or the extraction solvent. In some embodiments, a ketone is used as the extraction solvent. In some embodiments, one or more alkyl alkanoates such as methyl alkanoate esters are used as the organic solvent or the extraction solvent.

In some embodiments, the malonate feedstock is subjected to esterification conditions by first contacting the malonate feedstock with sulfuric acid or another acid, methanol or another alkanol, and o-xylene or toluene or another organic solvent, then subjecting the mixture to reactive extraction, followed by separation of the aqueous and organic layers.

In some embodiments, the molar ratio of sulfuric acid or another acid to ammonium malonate or another malonate salt is between 0.5:1.0 and 3.0:1.0. In some embodiments, the molar ratio of sulfuric acid to ammonium malonate is between 1.0:1.0 and 2.0:1.0. In some embodiments, the molar ratio of sulfuric acid to ammonium malonate is 1.0:1.0.

In some embodiments, the molar ratio of a lower alkanol such as methanol to a malonic acid salt such as ammonium malonate is between 1.0:1.0 and 15.0:1.0. In some embodiments, the molar ratio is between 2.0:1.0 and 12.0:1.0. In some embodiments, the molar ratio is between 3.0:1.0 and 10.0:1.0. In some embodiments, the molar ratio is 4.0:1.0.

In some embodiments, the mixture of malonate feedstock, the acid such as sulfuric acid, and the lower alkanol such as methanol is partitioned with an organic solvent such as o-xylene. In some embodiments, the volume ratio of the organic solvent such as o-xylene to malonate salt feedstock is between 1.0:1.0 and 5.0:1.0. In some embodiments, the volume ratio is between 2.0:1.0 and 3.0:1.0. In some embodiments, the volume ratio is 2.1:1.0.

In some embodiments, the mixture of malonate feedstock, the acid such as sulfuric acid, and the lower alkanol such as methanol is contacted with toluene. In some embodiments, the volume ratio of the organic solvent such as toluene to malonate feedstock is between 1.0:1.0 and 5.0:1.0. In some embodiments, the volume ratio is between 2.0:1.0 and 3.0:1.0. In some embodiments, the volume ratio is 2.1:1.0.

In some embodiments, the percent conversion to a dialkyl malonate, such as a di-lower alkyl malonate, preferably, DMM is between 80% and 100%. In some embodiments, the percent conversion to is between 85% and 95%. In some embodiments, the percent conversion to is 85%.

Illustrative and non-limiting examples of parameters for reactive extraction are listed in Table 5, below. Examples of estimated component concentrations from reactive extraction with 144 g/L DMM in extract are listed in Table 6, below.

TABLE 5
Reactive Extraction

| | |
|---|---|
| Toluene: malonate feedstock | 0.75 L toluene per L of aqueous feed |
| Liquid/liquid extraction | Scheibel column |
| equipment configuration | |
| Feed stages | Aqueous feed to top stage |
| | Toluene feed to bottom stage |
| Organic phase holdup | 33% |
| (fraction of internal volume) | |
| Number of stages | 8-14, optimally 12 |
| | [Designed to fit within a maximum height |
| | amenable to modular system, ~60 ft max] |
| Residence time for aqueous | 40 min |
| phase | |
| Total flux of both phases | (Specified by residence time and number |
| | of theoretical stages needed) |
| Aqueous feed temperature | 60° C.-90° C. |
| Solvent feed temperature | 60° C.-90° C. |
| Pressure | 0 psig or 30-50 psig |
| Equipment to separate | Coalescing mesh or In-process probe to |
| aqueous acid droplets from | avoid acid slipping into subsequent |
| extract | processes |
| Yield of total malonates in | 97 mol % (moles total malonates in |
| extract | extract/moles total malonates in aqueous |
| | feed) |
| Extract composition | 673 g/L toluene |
| | 144 g/L DMM |
| | 14 g/L MMM |
| | 37 g/L methanol |
| | 18 g/L water |
| | 3.5 g/L dimethyl succinate |
| | 0.5 g/L methyl acetate |
| | 0.2 g/L methyl pyruvate |
| Extract density | 0.89 kg/L at RT |
| Raffinate composition | 367 g/L water |
| | 357 g/L methanol |
| | 285 g/L sulfate ions and H₂SO₄ |
| | (as H₂SO₄) |
| | 34 g/L ammonium ions (as NH₃) |
| | 2.1 g/L DMM |
| | 1.9 g/L MMM |
| | 0.8 g/L malonic acid |
| | 0.4 g/L methyl pyruvate |
| | 0.5 g/L methyl acetate |
| | 0.4 g/L toluene |
| Raffinate density | 1.15 kg/L at RT |
| Materials of construction | Glass-lined |
| | Hastelloy impellers or PTFE lined |

TABLE 6
Reactive Extraction: Component Concentrations

| | | Estimated | |
|---|---|---|---|
| | | concentration | |
| Component | Area percent vs DMM | (g/L) | |
| Methyl acetate | 1.2% | 0.5 | g/L |
| | (includes some formed | | |
| | from MMM decomp. | | |
| | during analysis) | | |
| Methanol | 47.8% | 37 | g/L |
| Methyl pyruvate | 0.14% | 0.2 | g/L |
| DMM | 100% | 144 | g/L |
| Dimethyl succinate | 6.2% | 6.1 | g/L |
| Dimethyl fumarate | (these species are not | | |
| Methyl levulinate | distinguishable w/analytical | | |
| | method) | | |
| Methyl 3-methyl-2- | 0.02% | 0.029 | g/L |
| oxopentanoate | | | |
| Methyl | 0.02% | 0.018 | g/L |
| 3-methoxypropanoate | | | |
| Methyl | 0.01% | 0.014 | g/L |
| 2-oxobutanoate | | | |
| Dimethyl methylmalonate | 0.20% | 0.17 | g/L |
| Dimethyl sulfate | 0.002% | 0.0029 | g/L |
| Ethyl methyl malonate | co-elutes w/MMM | | |
| Dimethyl itaconate | 0.03% | 0.043 | g/L |

### Extract Stripping

In some embodiments, the methods disclosed herein include extract stripping. In some embodiments of this disclosure, the organic solvent employed in reactive extraction is stripped to remove water before the polishing esterification. This shifts the reaction equilibrium and allows a high yield of a dialkyl, such a di lower alkyl malonate, preferably, DMM to be obtained from the polishing esterification. In some embodiments, the lower alkanol such as methanol contained in the extract is removed along with water, and a significant amount of toluene is also stripped from the extract to achieve a low level of water in the stripped material. MMM or another mono alkyl malonate can thermally decompose around the atmospheric-pressure boiling point of an aromatic organic solvent such as toluene; so in some embodiments a moderate vacuum of ˜350 torr is used.

Illustrative and non-limiting parameters for extract stripping are shown in Table 7, below. Examples of rates of purified MMM thermal decomposition are listed in Table 8, below.

TABLE 7
Extract Stripping

| Water content after stripping | ≤0.02 | wt % |
|---|---|---|
| Fraction of toluene stripped | 10% | |
| Fraction of DMM stripped | ≤0.5% | |
| Pressure | 350 | torr |
| Condensate temperature | 46° | C. |
| Bottoms temperature | 90° | C. |
| Distillate composition estimate | 46 | wt % toluene |
| | 40 | wt % methanol |
| | 11 | wt % water |
| | 1.5 | wt % methyl acetate |
| | 0.03 | wt % methyl pyruvate |
| Distillate density estimate | 0.84 | kg/L |
| Stripped extract composition estimate | 77 | wt % toluene |
| | 20 | wt % DMM |
| | 2.0 | wt % MMM |
| | 0.7 | wt % dimethyl succinate |
| | 0.02 | wt % water |
| Density of stripped extract | 0.92 | kg/L |

| | |
|---|---|
| Materials of construction | SS316 |

TABLE 8
Extract Stripping: Rates of Purified
(99.1%) MMM Thermal Decomposition.

| | | | | MMM decomposition rate | |
|---|---|---|---|---|---|
| | | | | constant based on methyl | |
| Temperature | | Gravimetric loss rate | | acetate formation rate | |
| 80° | C. | 0.14% | h⁻¹ | 0.08% | h⁻¹ |
| 100° | C. | 1.6% | h⁻¹ | 1.2% | h⁻¹ |
| 120° | C. | 16% | h⁻¹ | 15.4% | h⁻¹ |

### Polishing Esterification

In some embodiments, the methods disclosed herein comprise polishing esterification. In some embodiments, the mono alkyl malonate, such as a mono lower alkyl malonate such as MMM in the organic extract is converted to the corresponding dialkyl malonate such as DMM. According to some embodiments, a lower alkanol corresponding to the mono alkyl malonate, e.g., methanol for MMM, is added to the stripped organic solvent or extract, which is then heated to 90° C. and fed to a fixed bed of acid resin in a downflow configuration. In some embodiments, water can be removed from the extract and additional alkanol added, to shift the reaction equilibrium towards the dialkyl malonate. In some embodiments, a solid acid catalyst can be used to catalyze the polishing esterification. In some embodiments, the malonates in the reactor effluent are present as 98.5 mol % of a dilkaly malonate such as DMM and 1.5 mol % of a mono alkyl malonate such as MMM. Illustrative and non-limiting parameters for polishing esterification are listed in Table 9, below.

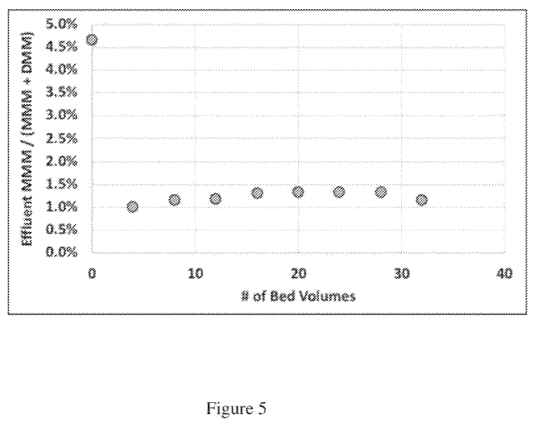

In some embodiments, as illustrated in FIG. 5, the polishing esterification takes place at 90° C. In some embodiments, LHSV (v/v/hr)=15, 20 mol MeOH/mol MMM in the feed. In some embodiments, the catalyst is Amberlyst-15. In some embodiments, the feed is an o-xylene extract. According to some embodiments, about one-third of the o-xylene in the extract is stripped prior to feeding to the fixed bed polishing reactor, which decreased water content of the feed to 0.02 wt %.

TABLE 9
Polishing Esterification

| Fresh methanol added | Add all methanol needed for entire process at this step | |
|---|---|---|
| | (~2.2 mol MeOH per mol malonate fed to plant, or | |
| | approx. 20 mol MeOH per mol MMM fed to the | |
| | polishing reactor) | |
| Catalyst | Amberlyst-15 | |
| LHSV | 15 | h⁻¹ |
| Temperature at inlet | 90° | C. |
| Adiabatic temperature rise | 1° | C. |
| Aspect ratio (height:diameter) | Min bed height 24 inches per Dow data sheet | |
| | Max pressure drop 15 psi across bed per Dow data sheet | |
| Malonate distribution in product | 98.5% | DMM |
| | 1.5% | MMM |

| | |
|---|---|
| Materials of construction | SS316 |

### Product Distillation

In some embodiments, the methods disclosed herein comprise product distillation. A mono alkyl malonate is decomposed to provide alkyl acetate, which alkyl acetate is removed with other low-boilers or low-boiling impurities. E.g., methyl acetate produced from MMM by decarboxylation can be separated with other low-boilers in a subsequent product stripping operation.

In some embodiments, low-boilers are removed from the organic extract product before high-boilers. In one non-limiting example, methyl acetate contamination of the product may be avoided by either or both of (a) operating at low enough pressure and temperature to limit mono alkyl malonate such as MMM decomposition; (b) partially condensing distillate product and allowing an alkyl acetate such as methyl acetate to slip past the condenser.

In some embodiments, the reaction mixture from the polishing esterification is purified by distillation. In some embodiments, the distillates are recycled back to the reactive extraction step.

In some embodiments, temperature is limited to minimize product decomposition and other side reactions. In some embodiments, a moderate vacuum pressure of 100 torr may enable an economical equipment design while limiting thermal exposure.

Illustrative and non-limiting parameters for product distillation are shown in Table 10, below. Examples of vapor pressure of DMM are listed in Table 11, below. Examples of Binary VLE data for DMM and dimethyl succinate are listed in Table 12, below. Examples of Binary VLE data for DMM and MMM are listed in Table 13, below. Examples of DMM 8-hour thermal decomposition are listed in Table 14, below.

TABLE 10
Product Distillation

| Dimethyl succinate:DMM ratio in distillate | ≤0.05 | wt % |
|---|---|---|
| DMM loss in bottoms | ≤1.0% of amount | |
| | fed to column | |
| Pressure | 100 | torr |
| Condensate temperature | 80° | C. |
| Bottoms temperature | 127° | C. |

TABLE 11
Vapor Pressure of DMM

| Temperature | Vapor pressure (mm Hg) |
|---|---|
| 120.0° C. | 122.2 |
| 140.0° C. | 249.5 |
| 160.0° C. | 502.8 |

TABLE 12
Binary VLE data for DMM and dimethyl succinate.

| Boiling | | DMM Mole | | | DMM K-Value | |
|---|---|---|---|---|---|---|
| point | Pressure | Fraction | | UNIFAC | Experi- | |
| ° C. | torr | Liquid | Vapor | Vapor | mental | UNIFAC |
| 120.0 | 69.5 | 0.19318 | 0.31605 | 0.29992 | 1.636 | 1.553 |
| 120.0 | 82.8 | 0.49818 | 0.66395 | 0.60697 | 1.333 | 1.218 |
| 119.8 | 102.4 | 0.90063 | 0.94576 | 0.92723 | 1.050 | 1.030 |
| 120.0 | 102.5 | 0.98055 | 0.98846 | 0.98592 | 1.008 | 1.005 |

TABLE 13
Binary VLE data for DMM and MMM.

| Boiling | | DMM Mole | | | DMM K-Value | |
|---|---|---|---|---|---|---|
| point | Pressure | Fraction | | UNIFAC | Experi- | |
| ° C. | torr | Liquid | Vapor | Vapor | mental | UNIFAC |
| 119.7 | 59.6 | 0.49789 | 0.96147 | 0.91103 | 1.93 | 1.83 |
| 120.0 | 77.1 | 0.70716 | 0.97688 | 0.95623 | 1.38 | 1.35 |
| 119.9 | 91.9 | 0.90257 | 0.99671 | 0.98555 | 1.10 | 1.09 |
| 119.9 | 103.4 | 0.97800 | 0.99829 | 0.99657 | 1.02 | 1.02 |

TABLE 14
DMM Thermal Decomposition.

| | | DMM decomposition rate based | |
|---|---|---|---|
| Temperature | Gravimetric loss | on decrease in area % purity | |
| 140° C. | 0.02% h⁻¹ | 0.05% | h⁻¹ |
| 160° C. | 0.02% h⁻¹ | 0.2% | h⁻¹ |

### Product Stripping

In some embodiments, the methods disclosed herein comprise product stripping. In some embodiments, in this step, low-boilers are stripped out of the DMM product. The low-boilers include but are not limited to toluene or another organic solvent, methanol or another lower alkanol, water, methyl acetate or another alkyl acetate, and methyl pyruvate or another alkyl pyruvate.

In some embodiments, during product stripping, temperature is limited to minimize product decomposition and other side reactions. In some embodiments, a moderate vacuum pressure of 100 torr can enable an economical equipment design while limiting thermal exposure.

In some embodiments, after being separated from the aqueous phase, the organic solvent or phase is stripped of solvent before undergoing a polishing esterification to complete conversion to di alkyl malonate such as DMM by contacting the stripped extract with fresh methanol, followed by distillation to purify the dialkyl malonate such as DMM.

In some embodiments, the percentage of lower alkanol such as methanol remaining in the mixture after stripping is between 0% and 5%. In some embodiments, the percentage of lower alkanol methanol remaining in the mixture after stripping is between 0% and 2%. In some embodiments, the percentage of methanol remaining in the mixture after stripping is 0%.

In some embodiments, the percentage of water remaining in the mixture after stripping is between 0% and 5%. In some embodiments, the percentage of water remaining in the mixture after stripping is between 0% and 2%. In some embodiments, the percentage of water remaining in the mixture after stripping is less than 1%.

In some embodiments, the molar ratio of the lower alkanol to a mono lower alkyl malonate, such as, methanol to MMM in the polishing esterification is between 5.0:1.0 and 25.0:1.0. In some embodiments, the molar ratio is between 10.0:1.0 and 20.0:1.0. In some embodiments, the molar ratio is 19.9:1.0.

In some embodiments, the percent conversion to a dialkyl malonate ester such as DMM during the polishing esterification is between 80% and 100%. In some embodiments, the percent conversion is between 85% and 100%. In some embodiments, the percent conversion is 86%.

Illustrative and non-limiting parameters for product stripping are shown in Table 15, below. Examples of Binary VLE data for DMM and toluene are listed in Table 16, below.

TABLE 15
Product Stripping

| Methyl pyruvate:DMM ratio in bottoms | ≤0.05 | wt % |
|---|---|---|
| Toluene:DMM ratio in bottoms | ≤0.05 | wt % |
| DMM loss in low-boiling distillate | ≤1.0% of amount fed to column | |
| Pressure | 100 | torr |
| Condensate temperature | 44° | C. |
| Bottoms temperature | 121° | C. |

TABLE 16
Binary VLE data for DMM and toluene

| Boiling | | DMM Mole | | | Toluene K-Value | |
|---|---|---|---|---|---|---|
| point | Pressure | Fraction | | UNIFAC | Experi- | |
| ° C. | torr | Liquid | Vapor | Vapor | mental | UNIFAC |
| 54.4 | 99.8 | 0.07952 | 0.01135 | 0.00362 | 1.07 | 1.08 |
| 59.7 | 99.8 | 0.28583 | 0.01832 | 0.01491 | 1.37 | 1.38 |
| 67.7 | 100.2 | 0.58234 | 0.03853 | 0.05268 | 2.30 | 2.27 |
| 83.0 | 99.9 | 0.82911 | 0.11597 | 0.19989 | 5.17 | 4.68 |
| 93.7 | 100.1 | 0.91362 | 0.24021 | 0.39266 | 8.80 | 7.03 |
| 112.9 | 99.6 | 0.97546 | 0.60473 | 0.74278 | 16.11 | 10.48 |
| 117.5 | 100.0 | 0.99180 | 0.91841 | 0.90284 | 9.95 | 11.84 |

### Raffinate Thermal Treatment

In some embodiments, the methods disclosed herein further comprise the treatment of a raffinate solution resulting from esterification or reactive extraction (see, for example, FIG. 3). In some embodiments, these methods comprise raffinate thermal treatment, such as thermal decomposition. In some embodiments, this step decarboxylates malonates such as malnic acid, or a di or mono protic malonate salt remaining in the raffinate, since they will not be stripped efficiently out of the aqueous AMS byproduct and may be undesirable in the byproduct. In some embodiments, the thermal decomposition of malonates produces carbon dioxide, alkyl acetate, and acetic acid.

In some embodiments, this thermal decomposition will proceed more rapidly under acidic conditions than under neutral pH conditions. In some embodiments, this step is performed on the raffinate from the reactive extraction process, before it is neutralized. In some embodiments, the decomposition occurs at 100° C. In some embodiments, the decomposition occurs above 100° C. In some embodiments, a sample is thermally treated after adjusting to pH=7 and decomposes at a moderate rate at 190° C. In some embodiments, neutral-pH decomposition occurs at temperatures higher than 190° C. In some embodiments, acidic decomposition occurs at temperatures greater than 100° C.

illustrative and non-limiting parameters for raffinate thermal treatment are shown in Table 17, below.

TABLE 17
Raffinate Thermal Treatment

| | | |
|---|---|---|
| Residual malonic acid in product | ≤0.5 | mg/L |
| Residual MMM in product | ≤0.5 | mg/L |
| Residual DMM in product | ≤0.5 | mg/L |
| Hold temperature/pressure for thermal treatment | 150° | C. |
| Estimated pressure at outlet of thermal treatment | 70 | psig |
| Liquid phase residence time for thermal treatment | 5 | minutes |

| | |
|---|---|
| Molar ratio of methyl acetate: acetic acid produced | 3.0 |
| Volume fraction gas at outlet of thermal treatment | 50% |

### Raffinate Neutralization

In some embodiments, after being separated from the organic phase following reactive extraction, the raffinate (the aqueous phase) is neutralized by contacting the aqueous phase with aqueous ammonium hydroxide, followed by concentration and stripping of solvent, after which the stripped solvent can be recycled back into the reactive extraction and the salt such as AMS can be collected. In some embodiments, ammonium hydroxide is added in this process step, to neutralize the raffinate before it is stripped. This can adjust the pH to a range in which the raffinate will not be corrosive to common materials of construction and can adjust the composition so that the sulfur: nitrogen ratio is within the appropriate range for the AMS byproduct. In some embodiments, the neutralization is exothermic and the heat of neutralization can be measured.

Illustrative and non-limiting parameters for raffinate neutralization are shown in Table 18, below.

TABLE 18
Raffinate Neutralization

| | |
|---|---|
| pH after neutralization | 7.0 |
| Ammonium: sulfate molar ratio after ammonium | 2.0 |
| hydroxide addition | |
| Amount of 25 wt % ammonium hydroxide to add | 0.26 kg/kg raffinate |

### Raffinate Stripping

In some embodiments, the methods disclosed herein comprise raffinate stripping. In some embodiments, after the raffinate from the reactive extraction has been thermally treated to decarboxylate residual malonates, and then neutralized with ammonium hydroxide, it is stripped to remove toluene, methanol, and other organics from the AMS byproduct stream.

Example parameters for raffinate stripping are shown in Table 19, below.

TABLE 19
Raffinate Stripping

| | | |
|---|---|---|
| Ammonium sulfate content of stripped raffinate | 40 | wt % |
| Water content of methanol distillate | 10 | wt % |
| Residual organics content in stripped raffinate | | |
| Methanol | ≤1 | mg/L |
| Toluene | ≤1 | mg/L |
| DMM | ≤1 | mg/L |
| MMM | ≤1 | mg/L |
| Malonic acid | ≤1 | mg/L |
| Methyl acetate | ≤1 | mg/L |
| Density of stripped raffinate | 1.15 | kg/L |

| | |
|---|---|
| Viscosity of stripped raffinate | 6 cP at 70° C. |

### Solvent Purification for Recycle

In some embodiments, the methods disclose herein comprise solvent purification for recycling. In some embodiments, toluene and methanol can be recycled in the process, which can allow for economical operation. The vast majority of the toluene used in the reactive extraction can be recovered in two streams: (1) the distillate from the water stripping operation prior to the polishing esterification, and (2) the distillate from stripping of the final dialkyl malonate, such as DMM. A significant amount of methanol can also be present in those distillates. In some embodiments, most of the methanol used in the reactive extraction ends up in the raffinate, and is recovered as distillate when the raffinate is stripped or in a solvent purification step (see for example, FIG. 3). It can be advantageous to purify such solvents later in the processing steps as opposed to earlier to avoid having to purify large volumes of material. In some embodiments, high-boiling impurities such as dimethyl succinate can be purged in the high-boiling waste during product distillation. In some embodiments, lower-boiling impurities that are generated in the process can also be purged from the process. In some embodiments, impurities boiling below the dialkyl malonate such as DMM can generally be expected to have a significant solubility in water, so it may be advantageous to purify the methanol recovered from the raffinate, and recycle the two distillate streams containing toluene and methanol directly to the reactive extraction. In some embodiments, a low-boiling impurity that may be generated in the process is methyl acetate, which can be stripped off of methanol and recovered as a distillate. In some embodiments, a high-boiling impurity that may be generated in the process is methyl pyruvate, which can be separated along with some water from methanol. In some embodiments, methyl pyruvate may be stripped out of the raffinate while the toluene and methanol are being stripped. In other embodiments, methyl pyruvate will can be purged from the solvent prior to recycle.

In some embodiments, recycled solvents and/or methanol are used in further reactive extraction processing (see, for example, FIG. 3).

### Product, By-Product, and Waste Stream

In some embodiments, the methods disclosed herein further comprise processing and analysis of product, by-product, and waste streams. Examples of parameters for DMM product are shown in Table 20, below. Examples of parameters for liquid AMS byproducts are shown in Table 21, below.

TABLE 20
DMM Product.

| DMM content | ≥99.75% | |
|---|---|---|
| Water content | ≤0.1 | wt % |
| Toluene content | ≤0.05 | wt % |
| Methyl pyruvate content | ≤0.05 | wt % |
| Dimethyl succinate content | ≤0.05 | wt % |
| Total acid number | ≤0.1 | mg KOH/g |
| Color | ≤20 | |
| Sulfur content | ≤0.1 | mg S/kg |

TABLE 21
Liquid AMS Byproduct

| AMS content | 40 | wt % |
|---|---|---|
| Molar ratio of NH₄:SO₄ | 2.0 | |
| Residual organics content | | |
| Methanol | ≤1 | mg/L |
| Toluene | ≤1 | mg/L |
| DMM | ≤1 | mg/L |
| MMM | ≤1 | mg/L |
| Malonic acid | ≤1 | mg/L |
| Methyl acetate | ≤1 | mg/L |

### Compositions

Further provided herein are compositions comprising malonic acid or diester of malonic acid. For determination of relative or absolute quantities of malonic acid and/or diester derivatives of malonic acid in any of the compositions described herein, any suitable analytical method may be used. For example, malonic acid and/or diester derivatives of malonic acid components of a composition may be quantified by chromatography such as liquid chromatography (for example, HPLC). Area per area percent (area %) of elution peaks associated with malonic acid and/or diester derivatives of malonic acid and/or their byproducts can be measured and quantified using known techniques, or weight per weight percent (w/w % or wt %) of each malonic acid and/or byproducts in a composition may be determined using known techniques for mass assay following HPLC analysis (for example, by using a standard malonic acid sample having a purity of greater than 99% (for example, 99.95% pure) as a reference). Malonic acid having a purity of 99.95% derived synthetically using non-renewable carbon may be purchased from Sigma-Aldrich, St. Louis, MO. Diester derivatives of malonic acid (DEM and DMM) having a purity of 98% and derived synthetically using non-renewable carbon may be purchased from Sigma-Aldrich, St. Louis, MO. For any of the compositions disclosed herein, quantities of malonic acid or diester derivatives of malonic acid are given as percentages refer to any of the wt %, area %, or vol %, unless specifically indicated otherwise.

Certain embodiments of the present disclosure relate to malonic acid compositions and/or diester derivatives of malonic acid compositions that are up to 100% bio-based as determined by ASTM International Radioisotope Method D 6866.

In certain embodiments, provided herein are malonic acid compositions (for example, malonate esters, diesters of malonic acid, DEM, and DMM) produced from malonates (for example, ammonium malonates and partially or fully deprotonated malonates). In certain embodiments, these compositions have a purity of about 90% or greater, for example, about 90%, 92%, 95%, 99%, or 99.5%, more than 95%, more than 96%, more than 97%, more than 98%, more than 99%, more than 99.95%, or more than 99.99%, or greater based on the total composition, where % refers to weight percent, area percent, or volume percent.

In some variations, the malonate may be produced by engineered microorganisms grown in media containing a renewable carbon source. The malonate ester compositions described herein are differentiated from malonate esters derived from chloroacetic acid and cyanide by the presence of substantially lower amounts of corresponding impurities.

In some embodiments, the methods provided herein result in diester derivatives of malonic acid containing DEM purity of at least 99% and less than 0.004% cyanoacetic acid.

In some embodiments, provided herein are compositions containing diester derivatives of malonic acid for use in polymerization reactions. In some such embodiments, the compositions contain diester derivatives of malonic acid with a low impurity level and use of these compositions may result in a less hazy polymer end-product. In some embodiments, provided herein are compositions containing diester derivatives of malonic acid for use as a blocking catalyst. In some embodiments, provided herein are compositions containing diester derivatives of malonic acid with a low impurity level and use of these compositions may result in a faster cure speed of resins and polymers. In some embodiments, provided herein are compositions containing diester derivatives of malonic acid with a low impurity level and use of these compositions may result in increased resin or polymer hardness.

In some embodiments, the process for purifying diester derivatives of malonic acid can result in compositions for use in downstream polymerization, crosslinking, Michael Addition reactions, or other such applications, such as those described in U.S. Pat. Nos. 9,718,988 and 9,834,701.

In some embodiments, the disclosed method comprises a modular system for the production of 26,000 MT/y of DMM product.

Additional components of compositions of diester derivatives of malonic acid resulting from the methods disclosed herein may comprise succinate diesters and general plasticizers. These components may be beneficial, or, may have no effect on the downstream uses of the compositions containing diester derivatives of malonic acid.

### Examples

### Example 1: a Recombinant P. Kudriavzevii Strain with Increased Malonate Titer

In this example, the P. kudriavzevii strain described in PCT Application Pub. No. WO 2015200545 (PCT App. No.: PCT US2015/037530, the entire contents of which are incorporated herein by this reference) is used to produce MA and/or MA salts. Methods on strain construction and culture requirements are also disclosed in this PCT application. Fermentation conditions for the production of MA and MA salts by this strain are described in Example 2 below. Practitioners in the art understand that other host cells may be considered for malonate production and that the recombinant P. kudriavzevii strain described here is a non-limiting example.

### Example 2: Fermentative Production of Malonic Acid by Recombinant P. Kudriavzevii

Consideration is preferably given to appropriate culture medium depending on the specific requirements of recombinant host cells, fermentation process and downstream purification processes. The media recipes disclosed herein are examples and can be modified as needed to suit individual fermentation goals and needs. More details on media recipes and fermentation conditions are described in the '545 publication (supra).
V01 solution comprised myo-inositol, thiamin hydrochloride, pyridoxial hydrochloride, nicotinic acid, calcium pantothenate, biotin, folic acid, p-aminobenzoic acid, and riboflavin.
T02 solution comprised citric acid monohydrate, H₃BO₃, CuSO₄-5H₂O, FeCl₃-6H₂O, sodium molybdate, and ZnSO₄-7H₂O.
T05 solution comprised citric acid monohydrate, H₃BO₃, CuSO₄-5H₂O, FeCl₃-6H₂O, sodium molybdate, and ZnSO₄-7H₂O.
S21 solution comprised KH₂PO₄, urea, and MgSO₄-7H₂O.
S24 solution comprised KH₂PO₄, and urea.
DE95 solution comprised approximately 70% (w/w) glucose solution. DE95 is a corn syrup equivalent that is commonly used in the industry.
HM PSA 24 medium comprised appropriate amounts of S21 solution, T05 solution, DE95 solution, and V01 solution.
HM PSA 25 medium comprised appropriate amounts of S21 solution, T05 solution, glucose, maltose, and V01 solution.
HF 22 medium comprised DE95 solution, KH₂PO₄, urea, T02 solution, and V01 solution.
HF 24 medium comprised glucose, maltose, KH₂PO₄, urea, T02 solution, and V01 solution.

In this example, recombinant P. kudriavzevii is used to produce MA. Each fermentation run is seeded from a single colony of recombinant P. kudriavzevii. Three separate, fed batch, fermentation runs are carried out; PSA 24 medium or PSA 25 medium is used as the batch medium, and HF 22 medium or HF 24 medium is used as the feed medium. The following parameters are common to all three fermentation runs: (1) 30° C. run temperature; (2) an impeller or agitator stir rate of 900-1,100 rpm (in some examples, the OUR is around 100-130 mmol/l/hr); (3) sterile air is blown into the fermenters at 1 1/min; (4) antifoam at manufacturer's recommended working concentrations; (5) run pH is maintained at around pH 5; (6) NaOH or NH₄OH is used to maintain fermentation pH at around pH 5; and (7) fermentation runs are about 53 hours long.

### Example 3: Ultrafiltration

In this example, ultrafiltration was carried out to remove high molecular high-molecular weight impurities that cause formation of stable emulsions during reactive extraction. Ultrafiltration prior to reactive extraction allows the reaction mixture to separate cleanly, as depicted in FIG. 9B (Vials 7 and 8). This Figure shows the layer separation. Vials 3, 4 and 5 were not subjected to ultrafiltration, whereas Vials 7 and 8 were (Vial 7: UF NaM o-xylene control; Vial 8: UF NaM methyl soyate control).

### Example 4: Reactive Extraction

In this example, reactive extraction was carried out in the presence of methanol and sulfuric acid of malonate feedstock that has been treated with strong acid cation exchange resin. The reaction kinetics and partition coefficients were determined and compared to the reactive extraction parameters of control ammonium malonate concentrate.

In this example, a 40 mL reaction vial was fitted with an ‘x’-shaped stir bar. Ion-exchanged ammonium malonate solution (3.06 M, pH 0.98) and 98% H₂SO₄ were added to the reaction vial, and it was submerged in an ice bath to control the exotherm of acidification. The mixture was then heated on a mixing reaction block to 70° C. Once heated, 99.5% methanol was injected at t=0 minutes. The reaction was sampled over the course of 60 minutes at various time points to monitor the composition of the reaction. Reaction sampling was performed using a 3 mL syringe fitted with a 2″ 21 g needle and pulling 1 mL of sample through the septum. To quench the reaction mixture, a saturated solution of sodium bicarbonate (4.5 mL) was used to quench the sample (0.5 mL) for a 10× dilution.

To determine partition coefficients, 15 mL of pre-reacted malonic acid concentrate (30 wt-% synthetic malonic acid (3.20 M, pH 0.72) or 50 wt-% synthetic malonic acid (5.74 M, pH 0.12)) was added to a 40 mL vial and heated to 68-70° C. internal with mixing at 1000 rpm. 11.25 mL 99.5% toluene (0.75 vol: vol) was added to the vial and mixed for 60 minutes to ensure equilibrium was reached. Mixing was stopped and 0.75 mL samples were taken from each phase and added to a chilled microcentrifuge tube. These samples were then diluted 10× within 30 seconds (aqueous phase was diluted in 4.5 mL saturated NaHCO₃; organic phase was diluted in 4.5 mL acetonitrile). Samples were analyzed via HPLC for malonate species concentrations.

In this example, a 40 mL reaction vial was fitted with an ‘x’-shaped stir bar. Ion-exchanged ammonium malonate solution (3.06 M, pH 0.98) and 98% H₂SO₄ were added to the reaction vial, and it was submerged in an ice bath to control the exotherm of acidification. The mixture was then heated on a mixing reaction block to 70° C. Once heated, 99.5% methanol was injected at t=0 minutes. The reaction was sampled over the course of 60 minutes at various time points to monitor the composition of the reaction. Reaction sampling was performed using a 3 mL syringe fitted with a 2″ 21g needle and pulling 1 mL of sample through the septum. To quench the reaction mixture, a saturated solution of sodium bicarbonate (4.5 mL) was used to quench the sample (0.5 mL) for a 10× dilution.

To determine partition coefficients, 15 mL of pre-reacted malonic acid concentrate (30 wt-% synthetic malonic acid (3.20 M, pH 0.72) or 50 wt-% synthetic malonic acid (5.74 M, pH 0.12)) was added to a 40 mL vial and heated to 68-70° C. internal with mixing at 1000 rpm. 11.25 mL 99.5% toluene (0.75 vol: vol) was added to the vial and mixed for 60 minutes to ensure equilibrium was reached. Mixing was stopped and 0.75 mL samples were taken from each phase and added to a chilled microcentrifuge tube. These samples were then diluted 10x within 30 seconds (aqueous phase was diluted in 4.5 mL saturated NaHCO₃; organic phase was diluted in 4.5 mL acetonitrile). Samples were analyzed via HPLC for malonate species concentrations.

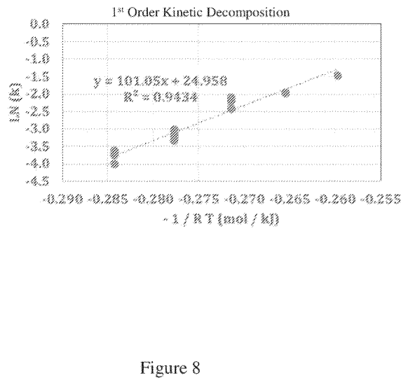

Table 22 and FIG. 8 show an example of the reaction composition of the 5.74 M malonic acid feed as well as the kinetic model fit to the measured reaction compositions.

TABLE 22
Pre-reaction mixture composition

| | | | MMM | DMM | MeOH | |
|---|---|---|---|---|---|---|
| Time | | MA conc | conc | conc | conc | H₂O conc |
| 0 | min | 1.77M | 0.00M | 0.00M | 15.98M | 10.27M |
| 3 | min | 0.48M | 0.94M | 0.35M | 14.35M | 11.91M |
| 5 | min | 0.29M | 0.95M | 0.55M | 13.94M | 12.31M |
| 10 | min | 0.12M | 0.78M | 0.88M | 13.45M | 12.81M |
| 20 | min | 0.06M | 0.63M | 1.08M | 13.19M | 13.06M |
| 40 | min | 0.05M | 0.58M | 1.16M | 13.09M | 13.16M |
| 60 | min | 0.05M | 0.56M | 1.17M | 13.07M | 13.18M |

TABLE 23
Equilibrium constants and H₂SO₄ loading at 70° C.

| | | | | | | | Post-rxn |
|---|---|---|---|---|---|---|---|
| | | | | | | | pH |
| | MA | MeOH | H2SO4 | | | Feed pH | (rel- |
| Source | Conc. | Eq. | Eq. | k₁ | k₂ | (relative) | ative) |
| 3.06M | 3.06 | 9 | 0.1 | 0.085 | 0.035 | 0.98 | 0.96 |
| MA | | | | | | | |
| 3.06M | 3.06 | 9 | 0.3 | 0.35 | 0.18 | 0.98 | 0.29 |
| MA | | | | | | | |
| 3.06M | 3.06 | 9 | 0.5 | 0.8 | 0.3 | 0.98 | 0 |
| MA | | | | | | | |
| Synthetic | 3.2 | 9 | 0.5 | 0.7 | 0.3 | 0.72 | 0 |
| Synthetic | 5.74 | 9 | 0.5 | 1.5 | 0.6 | 0.12 | 0 |
| NH4Mal | 3.65 | 9 | 2.2 | 1.75 | 0.6 | 5.10 | 0 |

Table 23 compares the rate constants of the ion exchanged 3.06 M MA feed, synthetic 3.2 M and 5.74 M MA feeds, as well as the control 3.65 M ammonium malonate batch feed. Only the 5.74 M MA feed at 0.5 mole eq H₂SO₄ was comparable to the control ammonium malonate reaction rates, with the 3.2 M MA feed at 0.5 mole eq H₂SO₄ at about half the rate.

The partition coefficients of MMM and DMM in toluene are compared in Table 24 for the control and the 3.2 M and 5.7 M MA feeds. The partition coefficients are about half that of the control ammonium malonate for both MMM and DMM for the 3.2 M MA feed. The 5.7 M MA feed is nearly the same partition for MMM with the DMM partition at half the control.

TABLE 24
Partition coefficients of MA, MMM, and DMM from
a pre-reaction mixture into toluene at 70° C.

| | | | Partition Coefficients | | | |
|---|---|---|---|---|---|---|
| | | | conc. in Org/conc. | | | Single |
| MA | MA | Toluene:Feed | in Aq (M org/M aq). | | | Stage |
| Source | Conc. | (vol:vol) | MA | MMM | DMM | Yield |
| NH₄Mal | 3.5M | 0.75 | 0 | 0.33 | 2.94 | 65.0% |
| Synthetic | 3.2M | 0.75 | 0 | 0.16 | 1.56 | 42.3% |
| Synthetic | 5.7M | 0.75 | 0 | 0.29 | 1.43 | 52.1% |

This example provides the reaction rates for conversion of malonic acid to mono-methyl malonate and DMM in malonic acid solutions at 3.2 M malonic acid (30 wt-%) and 5.7 M malonic acid (50 wt-%). These rates were determined at 0.1, 0.3, and 0.5 molar equivalents of sulfuric acid at 9 molar equivalents of methanol at 70° C. A 3.2 M malonic acid concentrate was found to have rates of 0.75-0.8 M⁻¹h⁻¹ and 0.3 M⁻¹h⁻¹ for k₁ and k₂ respectively. Increasing the feed concentration to 5.74 M malonic acid at 0.5 molar equivalents sulfuric acid achieved 1.5 M⁻¹h⁻¹ and 0.6 M⁻¹h⁻¹ for k₁ and k₂ respectively. This is compared to a control reaction at 1.75 M⁻¹h⁻¹ and 0.6 M⁻¹h⁻¹ for k₁ and k₂. The ion-exchanged material at 3.06 M malonic acid had comparable rate constants to the 3.2 M synthetic malonic acid feed, meaning that the solution should be nearly completely free of ammonium ions.

The partition coefficients of mono-methyl malonate and DMM between toluene and an aqueous phase were also compared to the control ammonium malonate feed. The synthetic 3.2 M malonic acid feed resulted in partition coefficients of 0.16 and 1.56 for MMM and DMM respectively. This result suggests that the ammonium bisulfate does generate a ‘salting-out’ effect on the MMM and DMM. The 5.7 M malonic acid feed had a higher partition for MMM at 0.29 and lower DMM partition at 1.43 than the 3.2 M malonic acid feed.

| | Run Name | | | | | | | |
|---|---|---|---|---|---|---|---|---|
| | 9 eq MeOH \| 0.75 Solvent: Feed \| 3 hours | | | | | | | |
| | Stream Name | | | | | | | |
| | Feed | | Solvent | | Raffinate | | Extract | |
| Density | 1.200 | g/mL | 0.877 | g/mL | 1.170 | g/mL | 0.900 | g/mL |
| Flow Rate | 188 | g/min | 101 | g/min | 183 | g/min | 119 | g/min |
| | 0.157 | L/min | 0.115 | L/min | 0.156 | L/min | 0.132 | L/min |

| | 0.198 moles | — | 0.022 moles | 0.1730 moles |
|---|---|---|---|---|
| | Malonate/min | | Malonate/min | Malonate/min |
| Yields | 10.9% is in raffinate | | | |
| | 87.6% yield in extract | | | |
| | 98.5% recovery | | | |
| Total | 289 g/min | | 302 g/min | |
| In/Out | | | | |

### Example 5: Thermal Decomposition

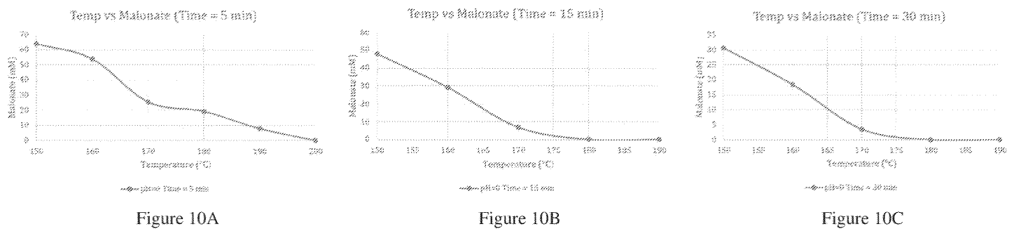

In this example of thermal decomposition, a model raffinate solution (pH=0) was tested at 6 different temperatures (150° C., 160° C., 170° C., 180° C., 190° C., or 200° C.) for three different lengths of time (5, 15, or 30 minutes). An LC column was used to measure the resulting malonic acid, acetic acid, MMM, and DMM. The feed composition was 76.7 mM malonic acid and 4.866 mM mono-methyl malonate. 2 mL of raffinate solution was added to each reaction vial containing a magnetic stir bar and heated in a reaction block. The reactions were stopped using a quench bucket. DMM was hydrolyzed immediately after addition. The total malonate in the model solution was 81.566 mM. The results for a five-minute (FIG. 10A), 15 minute (FIG. 10B), and 30 minute (FIG. 10C) experiments indicate that malonate fully decomposes at about 200° C. for 5 minutes. At T=15 minutes and T=30 minutes, complete decomposition starts at 180° C.

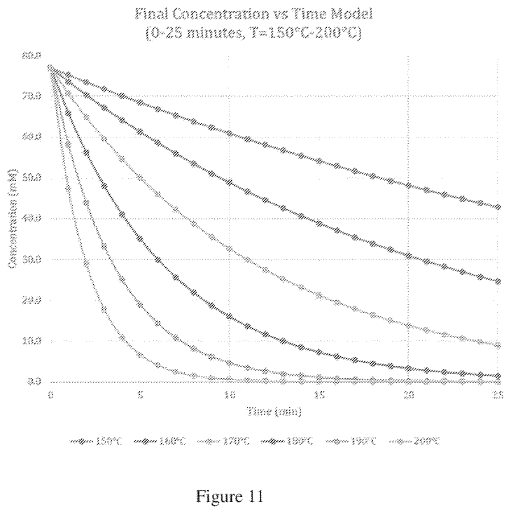

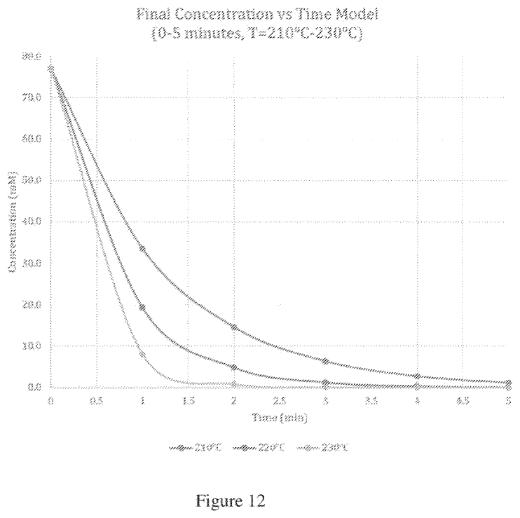

A first order kinetic decomposition plot was generated, as depicted in FIG. 8. The first order reaction best fits the data (with an R2=0.9434). Using the first order equation (y=101.05x+24.958), the kinetic rate law equation becomes: k=6.90E+10 exp (−101/RT). Using this equation, concentration vs time plots were generated to estimate the time needed to fully decompose malonates at different temperatures, as depicted in FIGS. 11 and 12.

FIG. 12 shows that at temperatures 150° C. to 180° C., malonates are fully decarboxylated after a residence time of t>60 minutes and, in addition, the rate slows down rapidly at higher residences times. At temperatures of 190° C. to 200° C., the decomposition takes off a bit with 190° C. requiring 25 minutes and 200° C. requiring 15 minutes for complete decarboxylation. FIG. 12 shows that at temperatures higher than 200° C., the required residence time for complete decarboxylation quickly dials down. The decomposition temperature versus the time to fully decarboxylate are depicted in Table 26.

TABLE 26
Decomposition Temperature vs. Decarboxylation Time

| Decomposition | Time to fully |
|---|---|
| Temperature | decarboxylate |
| (° C.) | (min) |
| 150 | 313 |
| 160 | 162 |
| 179 | 88 |
| 180 | 62 |
| 190 | 43 |
| 200 | 25 |
| 210 | 15 |
| 220 | 9 |
| 234 | 6 |
| 240 | 3 |
| 250 | 2 |

SEQUENCES

```
SEQ ID NO: 1. Wild type Saccharomyces cerevisiae EHD3 3-hydroxypropionyl-CoA hydrolase
amino acid sequence.
    1- MLRNTLKCAQ LSSKYGFKTT TRTFMTTQPQ LNVTDAPPVL
  41- FTVQDTARVI TLNRPKKLNA LNAEMSESMF KTLNEYAKSD
  81- TTNLVILKSS NRPRSFCAGG DVATVAIFNF NKEFAKSIKF
121- FTDEYSLNFQ IATYLKPIVT FMDGITMGGG VGLSIHTPFR
161- IATENTKWAM PEMDIGFFPD VGSTFALPRI VTLANSNSQM
201- ALYLCLTGEV VTGADAYMLG LASHYVSSEN LDALQKRLGE
241- ISPPFNNDPQ SAYFFGMVNE SIDEFVSPLP KDYVFKYSNE
281- KLNVIEACFN LSKNGTIEDI MNNLRQYEGS AEGKAFAQEI
321- KTKLLTKSPS SLQIALRLVQ ENSRDHIESA IKRDLYTAAN
361- MCMNQDSLVE FSEATKHKLI DKQRVPYPWT KKEQLFVSQL
401- TSITSPKPSL PMSLLRNTSN VTWTQYPYHS KYQLPTEQEI
441- AAYIEKRTND DTGAKVTERE VLNHFANVIP SRRGKLGIQS
481- LCKIVCERKC EEVNDGLRWK -500
SEQ ID NO: 2. Wild type Haemophilus influenzae nucleic acid sequence encoding YciA acyl-
CoA hydrolase.
    1- ATGTTTTACA CTGAAACTTA TGATGTGATT GTGATCGGTG
  41- GTGGTCATGC GGGTACAGAA GCCGCACTTG CACCAGCTCG
  81- TATGGGATTT AAAACCCTTT TATTAACACA TAATGTAGAT
121- ACTTTAGGGC AAATGTCTTG TAACCCTGCA ATTGGTGGGA
161- TCGGTAAAGG TCATTTAGTA AAAGAAGTAG ATGCAATGGG
201- CGGTTTAATG GCGCATGCTG CAGATAAAGC AGGGATCCAA
241- TTTCGTACTT TAAATAGCAG TAAAGGCCCA GCAGTGCGTG
281- CTACTCGAGC TCAAGCTGAC AGAGTTCTAT ATCGTCAAGC
321- TGTTCGTACT GCATTAGAAA ATCAACCTAA TTTAGATATT
361- TTCCAACAAG AAGCGACCGA TATTCTGATT AAGCAAGATC
401- GAGTTACAGG CGTTAGCACA AAAATGGGAT TAACTTTTCG
441- TGCTAAATCA GTGGTATTAA CTGCGGGTAC TTTCTTAGCT
481- GGTAAAATTC ATATTGGTTT GGAAAATTAT GAAGGTGGCC
521- GTGCAGGGGA TCCTGCTTCT GTAAATCTTT CACATCGATT
561- AAGAGATCTC GGATTACGTG TAGATCGCCT TAAAACAGGT
601- ACACCGCCGC GTATTGATGC ACGTACGATC AATTTTGATA
641- TTTTAGCTAA ACAACACGGT GATGCTGTTT TACCTGTGTT
681- TTCTTTTATG GGATCAGTTG ATGATCACCC TCAACAAATT
721- CCTTGTTATA TAACTCATAC CAATGAACAA ACCCATGAAG
761- TGATCCGTAA TAACTTGGAT CGCAGTCCAA TGTATACTGG
801- TGTGATTGAA GGGATCGGTC CACGTTATTG CCCATCCATT
841- GAAGATAAAG TGATGCGTTT CTCGGATCGT AATTCACATC
881- AAATTTATTT AGAACCAGAA GGCTTAACCA GTAATGAAGT
921- GTATCCAAAC GGGATCTCTA CCAGTTTACC GTTTGACGTG
961- CAAATGGGCA TTGTGAATTC TATGAAAGGT TTAGAAAACG -1000
SEQ ID NO: 3. EHD3 EC 3.1.2.4 malonyl-CoA hydrolase consensus sequence.
    1- ---------- ------MOBU UOB+AQ+UBB +----+----
  41- +GFOBBOB-- --+-----+B UOUUU----- ----------
  81- ---------- AQON+++UUU VOFUOQJUAR OOULNRPBKL
121- NALNUJMUJU OFBULNEYUK SJUUNOOOOB SUNQPRUOCA
161- GGDVAUOAO+ NOJBBF--BB UOJFFBUX₁YS ONFQOATYOK
201- POOOOMJGIT MGGGVGOUOH UPFROATENT BWAMPEMDIG
241- FFPDVGUUFA OPBOOUOANU BUQOAOYLCO TGJOOUGJJA
281- YOOGOASHYO UBJNOJJLJB RLGEOBPUJ+ OJ+-+++UQU
321- JJFFJOONJU OJEFUUP-OP BJYBFBYUNJ BLJVIJBCFJ
361- OUBOUUOBJO OBBLJJO-++ -YJGUJJABJ FABJOBJBLO
401- UKSPUSOQOA OBOOBJNUBJ BOJUAOBBDL OTAUNMCON-
441- +++QJUOOEF UJAUBBKLOJ KOBOPYPWBB B+JJOUOUQO
481- UUOOUPBPUO POULOBNUUN OTWBJYPBBO BYQLPUJUJO
521- BQYOBBBJNB N---+G-++O BOUBBJOOBB FUNONJUBBJ
561- KOGOJUOOBO OOJBBCUJJJ A+GGOBWB++ -+------ -598
SEQ ID NO: 4. Bacillus EC 3.1.2.4 malonyl-CoA hydrolase consensus sequence.
    1- MTEBVLFSOU JNGVAUITLN RPKALNSLSY JMLQPIGQKL
  41- KEWEBJJBIA OIVLKGAGUK GFCAGGDIKT LYEARSNEOA
  81- LQBAEBFFJE X₁YJIDTYOYQ YBKPIIACLD GIVMGGGVGL
121- TNGABYRIVT JBTKWAMPEM NIGFFPDVGA AYFLNBAPGY
161- UGBYVALUAU OLKAUDVLFI NAADYFMUUJ ULPBFLUJOJ
201- UONWBBJJJV BUBLKJOOBU FAUUUUOJUJ LUUOOEJONU
241- HFAFJUOEJI IBSLEBJQUU FAOBUBJULL SKSPOSLKVT
281- LKQFOJGBJK SOEJCFATDL OLAKNFMRHJ DFFEGVRSOV
321- ODKDQNPNYK YBQOUDVUJJ JVNBFFNLLN A -351
SEQ ID NO: 5. Pseudomonas EC 3.1.2.4 malonyl-CoA hydrolase consensus sequence.
    1- MNOBFEJBUU OBGARIGOAU LDAJBULNAL ULPMIJOLGJ
  41- BOBAWABJPG OOCVOLRGNG AKAFCAGGJV BBLOJACBJB
  81- PGJOPPLAAB FFAJX₁YRLJB BOHUYPKPOO CWGHGBVOGG
121- GMGLOQGAUO RIVTPUURLA MPEQUIGLYP DVGASWFLUR
161- OPGBLGLFOG LUGABONABD AODLJLADRF OBJBQQJJLO
201- JJLOQONWQE QUJOOLBSLO BAJJBBABJJ OPJAQOLPRR
241- QBODJOLDOA JOAUAWBAOJ AOBJBJDPLO ABAABBOBJG
281- CPOUABOVWJ QOBRARBLSL AJOFBMEYUO SLNCCRHPJF
321- UEGVRARLOD BDBQPBWBWP JOAQOPJAOO JAHFJBOWJG
361- BBPOAJOU++ -370
SEQ ID NO: 6. General bacterial EC 3.1.2.4 malonyl-CoA hydrolase consensus sequence.
    1- ---------- +--------- ---+------ M+M--TEHOO
  41- FUOSENGOAS IULNRPBALN SLUYDMOQPO GQBOBEWENJ
  81- EROALOOLB- GAGTBGFCAG GJOBUOYJAR SNEPG+ALQH
121- AERFFEJX₁YE OJTYOYQYKK POOACLDGIO MGGGVGLTNG
161- AKYBOOTERU BWAMPEMNIG FFPDVGAAYF LNBA------
201- PGYLGRYOAL UASIOKASDV OFONAAJYFM TSJSLPAFOT
241- EOESONWHKE DJOHTHLLKE +-+VORTFAT APNLJSEOAP
281- ----SLEEON SHFAF---DT OEEIW+AOHS OE--KJQSSF
321- ALKTKETOLS KUPOULBOTL KQFIDGRDKU OEJCFATJLV
361- OAKNFMBB-- --EJFFEGOB SVOODBJQNP NYBYKQOSDO
401- SJED------ ---------O NRFFNLONAG +H--PLADL+
441- ------++-- ---------- ---------- ----------
481- ---------- ---------- ----- -505
SEQ ID NO: 7. Artificial Sequence
    1- ccaatatata ataaaatatg gaggaatgcg atgctcagaa
  41- atacgctaaa atgtgcccaa -60
SEQ ID NO: 8. Artificial Sequence
    1- tgcctggaga tccttactcg agttggatcc ttatttccat
  41- cttaagccat cgttaacttc -60
SEQ ID NO: 9. Artificial Sequence
    1- ttttactgat gcgtattctt tgaattttca aatagca -37
SEQ ID NO: 10. Artificial Sequence
    1- tcaaagaata cgcatcagta aaaaatttga tgga -34
SEQ ID NO: 11. Artificial Sequence
    1- ttttactgat gtttattctt tgaattttca aatagcaact
  41- t -41
SEQ ID NO: 12. Artificial Sequence
    1- tcaaagaata aacatcagta aaaaatttga tggacttgg -39
SEQ ID NO: 13. Artificial Sequence
    1- ttttactgat tcgtattctt tgaattttca aatagcaac -39
SEQ ID NO: 14. Artificial Sequence
    1- tcaaagaata cgaatcagta aaaaatttga tggact -36
SEQ ID NO: 15. Artificial Sequence
    1- aattttttac tgatnnntat tctttgaatt ttcaaatagc -40
SEQ ID NO: 16. Artificial Sequence
    1- ttcaaagaat aannnatcag taaaaaattt gatggacttg -40
SEQ ID NO: 17. Artificial Sequence
    1- ccaatatata ataaaatatg gaggaatgcg atgtctacaa
  41- cacataacgt ccctc -55
SEQ ID NO: 18. Artificial Sequence
    1- tgcctggaga tccttactcg agttggatcc ttactcaaca
  41- ggtaaggcgc gag -53
SEQ ID NO: 19. Artificial Sequence
    1- ctaattacat gactcgaggt cgacggtatc gttatttcca
Tcttaagcca tcgttaactt c -61
SEQ ID NO: 20. Artificial Sequence
    1- cattagaaag aaagcatagc aatctaatct aagtttaaaa
  41- caatgactac tcaaccccag ctaaatg -67
SEQ ID NO: 21. Artificial Sequence
    1- gaaagcatag caatctaatc taagtttaaa acaatgaccg
  41- aacaagtctt attctcagta -60
SEQ ID NO: 22. Artificial Sequence
    1- ctaattacat gactcgaggt cgacggtatc gttaagcgtt
  41- caacaaattg aaaaatctg -59
SEQ ID NO: 23. Artificial Sequence
    1- gaaagcatag caatctaatc taagtttaaa acaatgaccg
  41- aacatgtatt attctcag -58
SEQ ID NO: 24. Artificial Sequence
    1- ctaattacat gactcgaggt cgacggtatc gttaagcgtt
  41- taacaaattg aaaaatc -57
SEQ ID NO: 25. Artificial Sequence
    1- gaaagcatag caatctaatc taagtttaaa acaatgactg
  41- aacacgtctt gttctctg -58
SEQ ID NO: 26. Artificial Sequence
    1- ctaattacat gactcgaggt cgacggtatc gttaagcgtt
  41- taacaaattg aaaaatctg -59
SEQ ID NO: 27. Artificial Sequence
    1- gaaagcatag caatctaatc taagtttaaa acaatgagaa
  41- gatacatcag aggtggt -57
SEQ ID NO: 28. Artificial Sequence
    1- ctaattacat gactcgaggt cgacggtatc gttatgcagc
  41- gttcaacaaa ttgaaaa -57
SEQ ID NO: 29. Artificial Sequence
    1- gaaagcatag caatctaatc taagtttaaa acaatgaccg
  41- aacaagtctt attctcag -58
SEQ ID NO: 30. Artificial Sequence
    1- ctaattacat gactcgaggt cgacggtatc gttaagcgtt
  41- caacaaattg aaaaatct -58
SEQ ID NO: 31. Artificial Sequence
    1- gaaagcatag caatctaatc taagtttaaa acaatgaact
  41- tacaatttga agaaagacca -60
SEQ ID NO: 32. Artificial Sequence
    1- ctaattacat gactcgaggt cgacggtatc gttacaaatc
  41- agctaaaggg tgttcac -57
SEQ ID NO: 33. Artificial Sequence
    1- gaaagcatag caatctaatc taagtttaaa acaatgaact
  41- tacactttga agaattgac -59
SEQ ID NO: 34. Artificial Sequence
    1- ctaattacat gactcgaggt cgacggtatc gttagtagtc
  41- agacaaatct gctaaag -57
SEQ ID NO: 35. Artificial Sequence
    1- gaaagcatag caatctaatc taagtttaaa acaatgacaa
  41- tccactgtga agtattaac -59
SEQ ID NO: 36. Artificial Sequence
    1- ctaattacat gactcgaggt cgacggtatc gttaaccaac
  41- gtcagccaaa gggtg -55
SEQ ID NO: 37. Artificial Sequence
    1- gaaagcatag caatctaatc taagtttaaa acaatgaatg
  41- tcacctttga agaaagag -58
SEQ ID NO: 38. Artificial Sequence
    1- ctaattacat gactcgaggt cgacggtatc gttatgccaa
  41- atcagctaaa gggtg -55
SEQ ID NO: 39. Artificial Sequence
    1- atgacgcaat ttgcatttgt gttccc -26
SEQ ID NO: 40. Artificial Sequence
    1- ttaaagctcg agcgccgct -19
```

What is claimed is:

1. A method comprising:
contacting an aqueous solution or aqueous mixture of a malonic acid salt, wherein the aqueous solution or the aqueous mixture of the malonic acid salt has a pH of about 2-about 9, or about 4-about 6, with:
a lower alkanol and
an acid
under conditions suitable to provide:
a diester of malonic acid and the lower alkanol, and optionally,
a monoester of malonic acid and the lower alkanol, and
extracting the diester into an organic solvent, preferentially over the monoester, if present, to provide the diester.

2. The method of claim 1, wherein the malonic acid salt comprises as cation: ammonium, primary ammonium, secondary ammonium, tertiary ammonium, quaternary ammonium, an alkali metal cation, an alkaline earth metal, or a mixture thereof.

3. The method of claim 1, wherein the malonic acid salt comprises an ammonium cation.

4. The method of claim 1, wherein the acid is a mineral acid or an acid resin.

5. The method of claim 1, wherein the acid is sulfuric acid.

6. The method of claim 1, wherein the lower alkanol is a C₁-C₃ alkanol.

7. The method of claim 1, wherein the lower alkanol is methyl alcohol or ethyl alcohol.

8. The method of claim 1, wherein the organic solvent is an aromatic solvent such as toluene or xylene, or an ester such as esters of monocarboxylic acids.

9. The method of claim 1, wherein the esterification is performed at a temperature greater than about 70° C. and less than about 100° C.

10. The method of claim 1, wherein the aqueous solution or mixture is obtained from a fermentation broth.

11. The method of claim 1, wherein the aqueous solution is obtained by ultrafiltration or nanofiltration of a fermentation broth comprising Pichia kudriavzevii cells.

12. A composition comprising:
more than about 95%, more than about 98%, or more than about 99% of malonic acid or a salt thereof and
the rest totaling up to 100% of one or more of lower alkyl levulinate, dialkyl 2-methylmalonic acid, monoalkyl malonamide (H₂N(O)CCH₂C(O)O—R), and dialkyl succinate.

13. The composition of claim 10, wherein the diester product is free of cyanoacetic acid or an ester thereof, or wherein the diester product contains less than 10 microgram/kg of cyanoacetic acid or an ester thereof.

14. The composition of claim 10, wherein the three carbons of the malonic acid core of the diester (HO₂C—CH₂—CO₂H) together has a ¹⁴C content of greater than 0.9 parts per trillion or are composed of more than 75% modern carbon, or more than 95% modern carbon, or substantially 100% modern carbon, as measured by standard ¹⁴C radioisotope measurements.

15. The composition of claim 10, wherein the composition is a solution, a mixture, or a solid.

16. A method for isolating a diester of malonic acid a from a fermentation broth, comprising:
separating fermentation medium from biomass by centrifugation;
filtering the fermentation medium via ultrafiltration or nanofiltration;
concentrating the filtered fermentation medium;
esterifying malonic acid present in the concentrated fermentation medium;
extracting mono and dialkyl ester of malonic acid from the concentrated fermentation medium via reactive extraction;
esterifying the extracted monoalkyl ester of malonic acid to the diester of malonic acid via polishing esterification; and
isolating the diester of malonic acid.

17. The method of claim 16, wherein the fermentation broth comprises an aqueous ammonium malonate.

18. The method of claim 16 or claim 17, wherein the centrifugation is carried out in two centrifugation steps.

19. The method of any one of claims 16-18, wherein the ultrafiltration or nanofiltration comprises a membrane having a nominal molecular weight cutoff <500,000 Da.

20. The method of any one of claims 16-19, wherein the reactive extraction uses a countercurrent extraction column.

21. The method of any one of claims 16-20, wherein the reactive extraction utilizes an organic solvent into which monoesters and diesters of malonic acid are extracted.

22. The method of claim 21, wherein the organic solvent comprises toluene, xylenes, o-xylene, anisole, a ketone, or an ester of a carboxylic acid such as an alkyl alkanoate ester.

23. The method of any one of claims 16-22 wherein the reactive extraction comprises a lower alkanol such as methanol or ethanol, and an acid such as sulfuric acid.

24. The method of any one of claims 16-23 wherein the temperature of the reactive extraction is about 30° C.- about 150° C. or greater than about 70° C. and less than about 100° C.

25. The method of any one of claims 16-24, wherein the fermentation broth comprises a malonic acid producing microorganism.

26. The method of claim 25, wherein the microorganism is a yeast selected from Saccharomyces and Pichia.

27. The method of claim 25 or claim 26 wherein the microorganism is Pichia kudriazevii.

28. A method for preparing a diester of malonic acid from a fermentation broth, comprising:
filtering a fermentation medium of a fermentation broth via ultrafiltration or nanofiltration, wherein the fermentation broth comprises a malonic acid salt;
converting the malonic acid salt into mono and diesters of malonic acid;
extracting into an organic solvent the diester of malonic acid preferentially over the mono ester of maloni acid via reactive extraction;
further converting the monoester of malonic acid present in the organic solvent to the diester of malnic acid by polishing esterfication.

29. The method of claim 28 further comprising prior to filtering, separating the fermentation broth into fermentation medium and biomass by centrifugation.

30. The method of claim 28 or 29 wherein the ultrafiltration or nanofiltration utilizes a membrane having a nominal molecular weight cutoff of <500,000 Da.

31. The method of any one of claims 28-30 wherein the reactive extraction uses a countercurrent extraction column.

32. The method of claim 28 wherein the organic solvent comprises an aromatic solvent such as toluene, xylene, o-xylene, anisole, or an ester of a carboxylic acid such as an alkyl alkanoate ester.

33. The method of any one of claims 28-32 wherein the reactive extraction comprises a lower alkanol and sulfuric acid.

34. The method of any one of claims 28-33 wherein the temperature of the reactive extraction is greater than about 70° C. and less than about 100° C.

35. The method of any one of claims 28-34, wherein the fermentation broth comprises a malonic acid-producing microorganism.

36. The method of claim 35, wherein the microorganism is a yeast selected from Saccharomyces and Pichia.

37. The method of claim 35 or claim 36 wherein the microorganism is Pichia kudriavzevii.

38. A method for preparing a diester of malonic acid from a fermentation broth, comprising:
filtering a fermentation medium of a fermentation broth via ultrafiltration or nanofiltration, wherein the fermentation broth comprises a malonic acid salt;
converting the malonic acid salt into diester and optionally monoester of malonic acid;
extracting into an organic solvent the diester of malonic acid preferentially over the monoester of malonic acid via reactive extraction;
washing the resulting organic phase with an alkaline aqueous solution to back-extract ionized monoester into the aqueous phase;
recycling the resulting aqueous phase to the salt conversion reactor; and
distilling the resulting organic phase to separate alcohol, solvent, and impurities from diester of malonic acid.

39. A method for preparing a diester of malonic acid from a fermentation broth, comprising:
filtering a fermentation medium of a fermentation broth via ultrafiltration or nanofiltration, wherein the fermentation broth comprises a malonic acid salt;
optionally, concentrating the salt by evaporation;
acidifying the salt using an acidic resin, which is periodically regenerated by washing with acid;
optionally, concentrating the aqueous malonic acid by evaporation;
esterifying the resulting aqueous malonic acid by a series of at least two esterification reactors containing acid resin, with water removed after each reaction stage and alcohol added before each reaction stage; and
distilling the resulting organic phase to separate alcohol, solvent, and impurities from diester of malonic acid.

40. The composition of claim 10, or the composition obtained by hydrolysis of malonate esters produced according to claim 10, wherein the percent modern carbon is greater than 75%, or greater than 95%, or is 100%, when measured using 14C radioisotope analysis corrected with standard methods such as delta 13C correction to correct for isotopic fractionation in the natural environment.
